(19) United States
(12) Patent Application Publication
(10) Pub. No.: US 2003/0224363 A1
(43) Pub. Date: Dec. 4, 2003

(54) Compositions and methods for modeling bacillus subtilis metabolism

(76) Inventors: Sung M. Park, San Diego, CA (US); Christophe H. Schilling, San Diego, CA (US); Bernhard O. Palsson, La Jolla, CA (US)

Correspondence Address: CAMPBELL & FLORES LLP, 7th Floor, 4370 La Jolla Village Drive, San Diego, CA, 92122, US

(21) Appl. No.: 10/102,022

(22) Filed: Mar. 19, 2002

(51) Int. Cl. C12Q 1/68; G06G 7/48; G06G 7/58; G06F 19/00; G01N 33/48; G01N 33/50

(52) U.S. Cl. 435/6; 702/20

(57) ABSTRACT

The invention provides an in silico model for determining a Bacillus subtilis physiological function. The model includes a data structure relating a plurality of B. subtilis reactants to a plurality of B. subtilis reactions, a constraint set for the plurality of B. subtilis reactions, and commands for determining a distribution of flux through the reactions that is predictive of a B. subtilis physiological function. A model of the invention can further include a gene database containing information characterizing the associated gene or genes. A regulated B. subtilis reaction can be represented in a model of the invention by including a variable constraint for the regulated reaction. The invention further provides methods for making an in silico B. subtilis model and methods for determining a B. subtilis physiological function using a model of the invention.

### BACKGROUND OF THE INVENTION

This invention relates generally to analysis of the activity of a chemical reaction network and, more specifically, to computational methods for simulating and predicting the activity of Bacillus subtilis reaction networks.

Members of the Bacillus genus are Gram-positive, endospore forming, rod-shaped bacteria found in soil and associated water sources. Bacillus subtilis, the type species of the genus, is a non-pathogenic organism that has been studied for many years as a model organism for many aspects of the biochemistry, genetics and physiology of Gram-positive bacteria, and also used to investigate the simple developmental process of sporulation. Research into B. subtilis has more recently been motivated by the widespread use of this organism in the production of industrially important products, including enzymes used in the food, brewing, dairy, textile and detergent industries, as well as nucleosides, antibiotics, vitamins and surfactants.

Over two-thirds of the world market of industrial enzymes is produced by Bacillus species. Commercially important enzymes made by Bacillus include proteases, amylases, glucanases and cellulases, which can be produced in abundance using simple media under industrial fermentation conditions. B. subtilis, and particularly protease-deficient strains, has also proven useful in the production of recombinant enzymes and proteins, including human growth factors.

Genetic manipulations, as well as changes in various fermentation conditions, are being considered in an attempt to improve the yield of industrially important products made by B. subtilis. However, these approaches are currently not guided by a clear understanding of how a change in a particular parameter, or combination of parameters, is likely to affect cellular behavior, such as the growth of the organism, the production of the desired product or the production of unwanted by-products. It would be valuable to be able to predict, how changes in fermentation conditions, such as an increase or decrease in the supply of oxygen or a media component, would affect cellular behavior and, therefore, fermentation performance. Likewise, before engineering the organism by the addition or deletion of one or more genes, it would be useful to be able to predict how these changes would affect cellular behavior.

However, it is currently difficult to make these sorts of predictions for B. subtilis because of the complexity of the metabolic reaction network that is encoded by the B. subtilis genome. Even relatively minor changes in media composition can affect hundreds of components of this network such that potentially hundreds of variables are worthy of consideration in making a prediction of fermentation behavior. Similarly, due to the complexity of interactions in the network, mutation of even a single gene can have effects on multiple components of the network. Thus, there exists a need for a model that describes B. subtilis reaction networks, such as its metabolic network, which can be used to simulate many different aspects of the cellular behavior of B. subtilis under different conditions. The present invention satisfies this need, and provides related advantages as well.

### SUMMARY OF THE INVENTION

The invention provides a computer readable medium or media, including: (a) a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating the substrate and the product, (b) a constraint set for the plurality of Bacillus subtilis reactions, and (c) commands for determining at least one flux distribution that minimizes or maximizes an objective function when the constraint set is applied to the data representation, wherein the at least one flux distribution is predictive of a Bacillus subtilis physiological function. In one embodiment, at least one of the Bacillus subtilis reactions in the data structure is annotated to indicate an associated gene and the computer readable medium or media further includes a gene database including information characterizing the associated gene. In another embodiment, at least one of the Bacillus subtilis reactions is a regulated reaction and the computer readable medium or media further includes a constraint set for the plurality of Bacillus subtilis reactions, wherein the constraint set includes a variable constraint for the regulated reaction.

The invention provides a method for predicting a Bacillus subtilis physiological function, including: (a) providing a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating the substrate and the product; (b) providing a constraint set for the plurality of Bacillus subtilis reactions; (c) providing an objective function, and (d) determining at least one flux distribution that minimizes or maximizes the objective function when the constraint set is applied to the data structure, thereby predicting a Bacillus subtilis physiological function. In one embodiment, at least one of the Bacillus subtilis reactions in the data structure is annotated to indicate an associated gene and the method predicts a Bacillus subtilis physiological function related to the gene.

The invention provides a method for predicting a Bacillus subtilis physiological function, including: (a) providing a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating the substrate and the product, wherein at least one of the Bacillus subtilis reactions is a regulated reaction; (b) providing a constraint set for the plurality of Bacillus subtilis reactions, wherein the constraint set includes a variable constraint for the regulated reaction; (c) providing a condition-dependent value to the variable constraint; (d) providing an objective function, and (e) determining at least one flux distribution that minimizes or maximizes the objective function when the constraint set is applied to the data structure, thereby predicting a Bacillus subtilis physiological function.

Also provided by the invention is a method for making a data structure relating a plurality of Bacillus subtilis reactants to a plurality of Bacillus subtilis reactions in a computer readable medium or media, including: (a) identifying a plurality of Bacillus subtilis reactions and a plurality of Bacillus subtilis reactants that are substrates and products of the Bacillus subtilis reactions; (b) relating the plurality of Bacillus subtilis reactants to the plurality of Bacillus subtilis reactions in a data structure, wherein each of the Bacillus subtilis reactions includes a reactant identified as a substrate of the reaction, a reactant identified as a product of the reaction and a stoichiometric coefficient relating the substrate and the product; (c) determining a constraint set for the plurality of Bacillus subtilis reactions; (d) providing an objective function; (e) determining at least one flux distribution that minimizes or maximizes the objective function when the constraint set is applied to the data structure, and (f) if the at least one flux distribution is not predictive of a Bacillus subtilis physiological function, then adding a reaction to or deleting a reaction from the data structure and repeating step (e), if the at least one flux distribution is predictive of a Bacillus subtilis physiological function, then storing the data structure in a computer readable medium or media. The invention further provides a data structure relating a plurality of Bacillus subtilis reactants to a plurality of Bacillus subtilis reactions, wherein the data structure is produced by the method.

### BRIEF DESCRIPTION OF THE DRAWINGS

FIG. 1 shows contour diagrams for glucose uptake (A and D), oxygen uptake (B and E), and carbon dioxide evolution (C and F) rates as a function of ratio of ATP molecules produced per atom of oxygen (PO ratio) and ATP maintenance requirement. The data from Tables 1 and 2 were used as inputs to the system. Growth rates are fixed at 0.11 hr⁻¹ (A-C) or 0.44 hr⁻¹ (D-F).

FIG. 2 shows phase plane analysis for possible byproduct patterns under different oxygen and glucose uptake rates. Units are in mmol/g dry cell weight (DCW)/hr. Depending on which byproducts are allowed to be secreted, different phase planes can be formed. Panel A: Acetate, acetoin, and diacetoin are allowed. Panel B: Butanediol, acetate, acetoin, and diacetoin are allowed. Panel C: Lactate (or ethanol), acetate, acetoin, and diacetoin are allowed. Thin lines in the upper and middle panels are isoclines that represent the locus of points in the two-dimensional space that define the same value of the objective function.

FIG. 3 shows maximum yield graphs for riboflavin (A), subtilisin (B), and amylase (C) as a function of growth rate and PO ratio.

FIG. 4 shows, in part A, carbon flux distributions that maximize biomass, riboflavin, amylase or protease (top, second, third and bottom numbers, respectively, in boxes) production in B. subtilis on glucose as the carbon substrate and ammonia as the nitrogen substrate, and, in part B, carbon flux distributions that maximize riboflavin biosynthesis as a function of PO ratio of 0.5, 1.0 and 1.5 (top, second and bottom numbers, respectively, in boxes).

FIG. 5 shows a schematic representation of a hypothetical metabolic network.

FIG. 6 shows mass balance constraints and flux constraints (reversibility constraints) that can be placed on the hypothetical metabolic network shown in FIG. 5.

FIG. 7 shows the stoichiometric matrix (S) for the hypothetical metabolic network shown in FIG. 5.

FIG. 8 shows a balanced pathway for histidine utilization in B. subtilis.

FIG. 9 shows a flux distribution map comparing results for simulation with a stand-alone metabolic model (lower numbers) and a combined regulatory/metabolic model (upper numbers).

FIG. 10 shows two possible routes for the synthesis of UDP-N-acetylglucosamine.

FIG. 11 shows, in Panel A, an exemplary biochemical reaction network and in Panel B, an exemplary regulatory control structure for the reaction network in panel A.

### DETAILED DESCRIPTION OF THE INVENTION

The present invention provides an in silico B. subtilis model that describes the interconnections between the metabolic genes in the B. subtilis genome and their associated reactions and reactants. The model can be used to simulate different aspects of the cellular behavior of B. subtilis under different environmental and genetic conditions, thereby providing valuable information for industrial and research applications. An advantage of the model of the invention is that it provides a holistic approach to simulating and predicting the metabolic activity of B. subtilis.

As an example, the B. subtilis metabolic model can be used to determine the optimal conditions for fermentation performance, such as for maximizing the yield of a specific industrially important enzyme. The model can also be used to calculate the range of cellular behaviors that B. subtilis can display as a function of variations in the activity of one gene or multiple genes. Thus, the model can be used to guide the design of improved fermentation conditions and organismal genetic makeup for a desired application. This ability to make predictions regarding cellular behavior as a consequence of altering specific parameters will increase the speed and efficiency of industrial development of B. subtilis strains and conditions for their use.

The B. subtilis metabolic model can also be used to predict or validate the assignment of particular biochemical reactions to the enzyme-encoding genes found in the genome, and to identify the presence of reactions or pathways not indicated by current genomic data. Thus, the model can be used to guide the research and discovery process, potentially leading to the identification of new enzymes, medicines or metabolites of commercial importance.

The models of the invention are based on a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating the substrate and the product.

As used herein, the term “Bacillus subtilis reaction” is intended to mean a conversion that consumes a substrate or forms a product that occurs in or by a viable strain of Bacillus subtilis. The term can include a conversion that occurs due to the activity of one or more enzymes that are genetically encoded by a Bacillus subtilis genome. The term can also include a conversion that occurs spontaneously in a Bacillus subtilis cell. Conversions included in the term include, for example, changes in chemical composition such as those due to nucleophilic or electrophilic addition, nucleophilic or electrophilic substitution, elimination, isomerization, deamination, phosphorylation, methylation, reduction, oxidation or changes in location such as those that occur due to a transport reaction that moves a reactant from one cellular compartment to another. In the case of a transport reaction, the substrate and product of the reaction can be chemically the same and the substrate and product can be differentiated according to location in a particular cellular compartment. Thus, a reaction that transports a chemically unchanged reactant from a first compartment to a second compartment has as its substrate the reactant in the first compartment and as its product the reactant in the second compartment. It will be understood that when used in reference to an in silico model or data structure, a reaction is intended to be a representation of a chemical conversion that consumes a substrate or produces a product.

As used herein, the term “Bacillus subtilis reactant” is intended to mean a chemical that is a substrate or a product of a reaction that occurs in or by a viable strain of Bacillus subtilis. The term can include substrates or products of reactions performed by one or more enzymes encoded by a Bacillus subtilis genome, reactions occurring in Bacillus subtilis that are performed by one or more non-genetically encoded macromolecule, protein or enzyme, or reactions that occur spontaneously in a Bacillus subtilis cell. Metabolites are understood to be reactants within the meaning of the term. It will be understood that when used in reference to an in silico model or data structure, a reactant is intended to be a representation of a chemical that is a substrate or a product of a reaction that occurs in or by a viable strain of Bacillus subtilis.

As used herein the term “substrate” is intended to mean a reactant that can be converted to one or more products by a reaction. The term can include, for example, a reactant that is to be chemically changed due to nucleophilic or electrophilic addition, nucleophilic or electrophilic substitution, elimination, isomerization, deamination, phosphorylation, methylation, reduction, oxidation or that is to change location such as by being transported across a membrane or to a different compartment.

As used herein, the term “product” is intended to mean a reactant that results from a reaction with one or more substrates. The term can include, for example, a reactant that has been chemically changed due to nucleophilic or electrophilic addition, nucleophilic or electrophilic substitution, elimination, isomerization, deamination, phosphorylation, methylation, reduction or oxidation or that has changed location such as by being transported across a membrane or to a different compartment.

As used herein, the term “stoichiometric coefficient” is intended to mean a numerical constant correlating the number of one or more reactants and the number of one or more products in a chemical reaction. Typically, the numbers are integers as they denote the number of molecules of each reactant in an elementally balanced chemical equation that describes the corresponding conversion. However, in some cases the numbers can take on non-integer values, for example, when used in a lumped reaction or to reflect empirical data.

As used herein, the term “plurality,” when used in reference to Bacillus subtilis reactions or reactants, is intended to mean at least 2 reactions or reactants. The term can include any number of Bacillus subtilis reactions or reactants in the range from 2 to the number of naturally occurring reactants or reactions for a particular strain of Bacillus subtilis. Thus, the term can include, for example, at least 10, 20, 30, 50, 100, 150, 200, 300, 400, 500, 600 or more reactions or reactants. The number of reactions or reactants can be expressed as a portion of the total number of naturally occurring reactions for a particular strain of Bacillus subtilis such as at least 20%, 30%, 50%, 60%, 75%, 90%, 95% or 98% of the total number of naturally occurring reactions that occur in a particular strain of Bacillus subtilis.

As used herein, the term “data structure” is intended to mean a physical or logical relationship among data elements, designed to support specific data manipulation functions. The term can include, for example, a list of data elements that can be added combined or otherwise manipulated such as a list of representations for reactions from which reactants can be related in a matrix or network. The term can also include, a matrix that correlates data elements from two or more lists of information such as a matrix that correlates reactants to reactions. Information included in the term can represent, for example, a substrate or product of a chemical reaction, a chemical reaction relating one or more substrates to one or more products, a constraint placed on a reaction, or a stoichiometric coefficient.

As used herein, the term “constraint” is intended to mean an upper or lower boundary for a reaction. A boundary can specify a minimum or maximum flow of mass, electrons or energy through a reaction. A boundary can further specify directionality of a reaction. A boundary can be a constant value such as zero, infinity, or a numerical value such as an integer. Alternatively, a boundary can be a variable boundary value as set forth below.

As used herein, the term “variable,” when used in reference to a constraint is intended to mean capable of assuming any of a set of values in response to being acted upon by a constraint function. The term “function,” when used in the context of a constraint, is intended to be consistent with the meaning of the term as it is understood in the computer and mathematical arts. A function can be binary such that changes correspond to a reaction being off or on. Alternatively, continuous functions can be used such that changes in boundary values correspond to increases or decreases in activity. Such increases or decreases can also be binned or effectively digitized by a function capable of converting sets of values to discreet integer values. A function included in the term can correlate a boundary value with the presence, absence or amount of a biochemical reaction network participant such as a reactant, reaction, enzyme or gene. A function included in the term can correlate a boundary value with an outcome of at least one reaction in a reaction network that includes the reaction that is constrained by the boundary limit. A function included in the term can also correlate a boundary value with an environmental condition such as time, pH, temperature or redox potential.

As used herein, the term “activity,” when used in reference to a reaction, is intended to mean the amount of product produced by the reaction, the amount of substrate consumed by the reaction or the rate at which a product is produced or a substrate is consumed. The amount of product produced by the reaction, the amount of substrate consumed by the reaction or the rate at which a product is produced or a substrate is consumed can also be referred to as the flux for the reaction.

As used herein, the term “activity,” when used in reference to Bacillus subtilis, is intended to mean the magnitude or rate of a change from an initial state of Bacillus subtilis to a final state of Bacillus subtilis. The term can include the amount of a chemical consumed or produced by Bacillus subtilis, the rate at which a chemical is consumed or produced by Bacillus subtilis, the amount or rate of growth of Bacillus subtilis or the amount of or rate at which energy, mass or electrons flow through a particular subset of reactions.

The invention provides a computer readable medium, having a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating the substrate and the product. The plurality of Bacillus subtilis reactions can include reactions of a peripheral metabolic pathway.

As used herein, the term “peripheral,” when used in reference to a metabolic pathway, is intended to mean a metabolic pathway that includes one or more reactions that are not a part of a central metabolic pathway. As used herein, the term “central,” when used in reference to a metabolic pathway, is intended to mean a metabolic pathway selected from glycolysis, the pentose phosphate pathway (PPP), the tricarboxylic acid (TCA) cycle and the electron transfer system (ETS) and associated anapleurotic reactions.

A plurality of Bacillus subtilis reactants can be related to a plurality of Bacillus subtilis reactions in any data structure that represents, for each reactant, the reactions by which it is consumed or produced. Thus, the data structure, which is referred to herein as a “reaction network data structure,” serves as a representation of a biological reaction network or system. An example of a reaction network that can be represented in a reaction network data structure of the invention is the collection of reactions that constitute the metabolic reactions of B. subtilis.

The methods and models of the invention can be applied to any strain of Bacillus subtilis including, for example, strain 168 or any laboratory or production strain. A strain of Bacillus subtilis can be identified according to classification criteria known in the art. Those skilled in the art will be able to recognize a strain as a Bacillus subtilis because it will have characteristics that are closer to known strains of Bacillus subtilis than to strains of other organisms. Such characteristics can include, for example, classical microbiological characteristics, such as those upon which taxonomic classification is traditionally based, or evolutionary distance as determined for example by comparing sequences from within the genomes of organisms, such as ribosome sequences.

The reactants to be used in a reaction network data structure of the invention can be obtained from or stored in a compound database. As used herein, the term “compound database” is intended to mean a computer readable medium or media containing a plurality of molecules that includes substrates and products of biological reactions. The plurality of molecules can include molecules found in multiple organisms, thereby constituting a universal compound database. Alternatively, the plurality of molecules can be limited to those that occur in a particular organism, thereby constituting an organism-specific compound database. Each reactant in a compound database can be identified according to the chemical species and the cellular compartment in which it is present. Thus, for example, a distinction can be made between glucose in the extracellular compartment versus glucose in the cytosol. Additionally each of the reactants can be specified as a metabolite of a primary or secondary metabolic pathway. Although identification of a reactant as a metabolite of a primary or secondary metabolic pathway does not indicate any chemical distinction between the reactants in a reaction, such a designation can assist in visual representations of large networks of reactions.

As used herein, the term “compartment” is intended to mean a subdivided region containing at least one reactant, such that the reactant is separated from at least one other reactant in a second region. A subdivided region included in the term can be correlated with a subdivided region of a cell. Thus, a subdivided region included in the term can be, for example, the intracellular space of a cell; the extracellular space around a cell; the periplasmic space, the interior space of an organelle such as a mitochondrium, endoplasmic reticulum, Golgi apparatus, vacuole or nucleus; or any subcellular space that is separated from another by a membrane or other physical barrier. Subdivided regions can also be made in order to create virtual boundaries in a reaction network that are not correlated with physical barriers. Virtual boundaries can be made for the purpose of segmenting the reactions in a network into different compartments or substructures.

As used herein, the term “substructure” is intended to mean a portion of the information in a data structure that is separated from other information in the data structure such that the portion of information can be separately manipulated or analyzed. The term can include portions subdivided according to a biological function including, for example, information relevant to a particular metabolic pathway such as an internal flux pathway, exchange flux pathway, central metabolic pathway, peripheral metabolic pathway, or secondary metabolic pathway. The term can include portions subdivided according to computational or mathematical principles that allow for a particular type of analysis or manipulation of the data structure.

The reactions included in a reaction network data structure can be obtained from a metabolic reaction database that includes the substrates, products, and stoichiometry of a plurality of metabolic reactions of Bacillus subtilis. The reactants in a reaction network data structure can be designated as either substrates or products of a particular reaction, each with a stoichiometric coefficient assigned to it to describe the chemical conversion taking place in the reaction. Each reaction is also described as occurring in either a reversible or irreversible direction. Reversible reactions can either be represented as one reaction that operates in both the forward and reverse direction or be decomposed into two irreversible reactions, one corresponding to the forward reaction and the other corresponding to the backward reaction.

Reactions included in a reaction network data structure can include intra-system or exchange reactions. Intra-system reactions are the chemically and electrically balanced interconversions of chemical species and transport processes, which serve to replenish or drain the relative amounts of certain metabolites. These intra-system reactions can be classified as either being transformations or translocations. A transformation is a reaction that contains distinct sets of compounds as substrates and products, while a translocation contains reactants located in different compartments. Thus a reaction that simply transports a metabolite from the extracellular environment to the cytosol, without changing its chemical composition is solely classified as a translocation, while a reaction such as the phosphotransferase system (PTS) which takes extracellular glucose and converts it into cytosolic glucose-6-phosphate is a translocation and a transformation.

Exchange reactions are those which constitute sources and sinks, allowing the passage of metabolites into and out of a compartment or across a hypothetical system boundary. These reactions are included in a model for simulation purposes and represent the metabolic demands placed on B. subtilis. While they may be chemically balanced in certain cases, they are typically not balanced and can often have only a single substrate or product. As a matter of convention the exchange reactions are further classified into demand exchange and input/output exchange reactions.

The metabolic demands placed on the B. subtilis metabolic reaction network can be readily determined from the dry weight composition of the cell which is available in the published literature or which can be determined experimentally. The uptake rates and maintenance requirements for B. subtilis can be determined by microbiological experiments in which the uptake rate is determined by measuring the depletion of the substrate from the growth medium. The measurement of the biomass at each point can also be determined, in order to determine the uptake rate per unit biomass. The maintenance requirements can be determined from a chemostat experiment. The glucose uptake rate is plotted versus the growth rate, and the y-intercept is interpreted as the non-growth associated maintenance requirements. The growth associated maintenance requirements are determined by fitting the model results to the experimentally determined points in the growth rate versus glucose uptake rate plot.

Input/output exchange reactions are used to allow extracellular reactants to enter or exit the reaction network represented by a model of the invention. For each of the extracellular metabolites a corresponding input/output exchange reaction can be created. These reactions are always reversible with the metabolite indicated as a substrate with a stoichiometric coefficient of one and no products produced by the reaction. This particular convention is adopted to allow the reaction to take on a positive flux value (activity level) when the metabolite is being produced or removed from the reaction network and a negative flux value when the metabolite is being consumed or introduced into the reaction network. These reactions will be further constrained during the course of a simulation to specify exactly which metabolites are available to the cell and which can be excreted by the cell.

A demand exchange reaction is always specified as an irreversible reaction containing at least one substrate. These reactions are typically formulated to represent the production of an intracellular metabolite by the metabolic network or the aggregate production of many reactants in balanced ratios such as in the representation of a reaction that leads to biomass formation, also referred to as growth. As set forth in the Examples, the biomass components to be produced for growth include the components listed in Table 3 and ALA, ARG, ASP, ASN, CYS, GLU, GLN, GLY, HIS, ILE, LEU, LYS, MET, PHE, PRO, THR, TRP, TYR, VAL, DATP, DGTP, DCTP, DTTP, GTP, CTP, UTP, PEPTIDO, PS, PE, CL, PG, THIAMIN, GLYTC1, GLYTC2, TEICHU, MTHF, SUCCOA, PTRC, Q, HEMEA, SHEME, FAD, NADP and SPMD.

A demand exchange reactions can be introduced for any metabolite in a model of the invention. Most commonly these reactions are introduced for metabolites that are required to be produced by the cell for the purposes of creating a new cell such as amino acids, nucleotides, phospholipids, and other biomass constituents, or metabolites that are to be produced for alternative purposes. Once these metabolites are identified, a demand exchange reaction that is irreversible and specifies the metabolite as a substrate with a stoichiometric coefficient of unity can be created. With these specifications, if the reaction is active it leads to the net production of the metabolite by the system meeting potential production demands. Examples of processes that can be represented as a demand exchange reaction in a reaction network data structure and analyzed by the methods of the invention include, for example, production or secretion of an individual protein; production or secretion of an individual metabolite such as an amino acid, vitamin, nucleoside, antibiotic or surfactant; production of ATP for extraneous energy requiring processes such as locomotion; or formation of biomass constituents.

In addition to these demand exchange reactions that are placed on individual metabolites, demand exchange reactions that utilize multiple metabolites in defined stoichiometric ratios can be introduced. These reactions are referred to as aggregate demand exchange reactions. An example of an aggregate demand reaction is a reaction used to simulate the concurrent growth demands or production requirements associated with cell growth that are placed on a cell, for example, by simulating the formation of multiple biomass constituents simultaneously at a particular cellular growth rate.

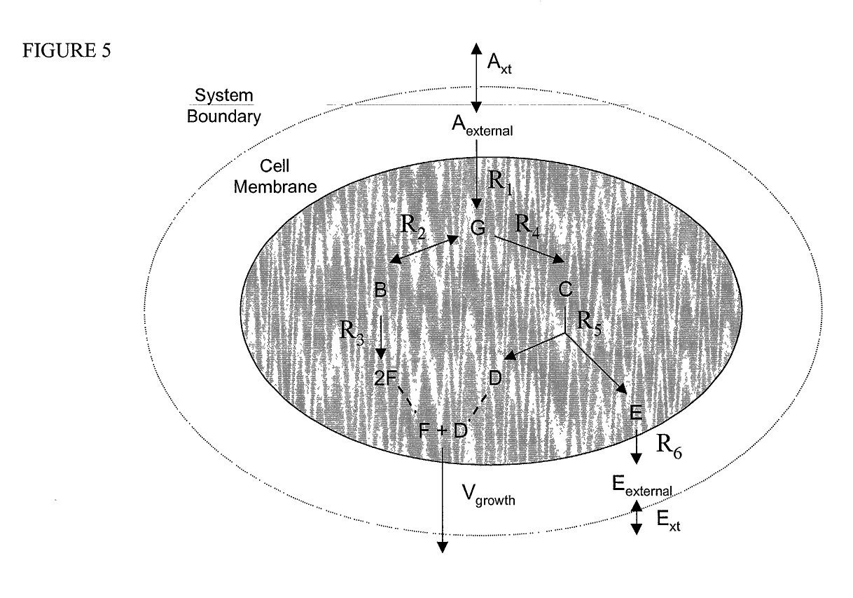

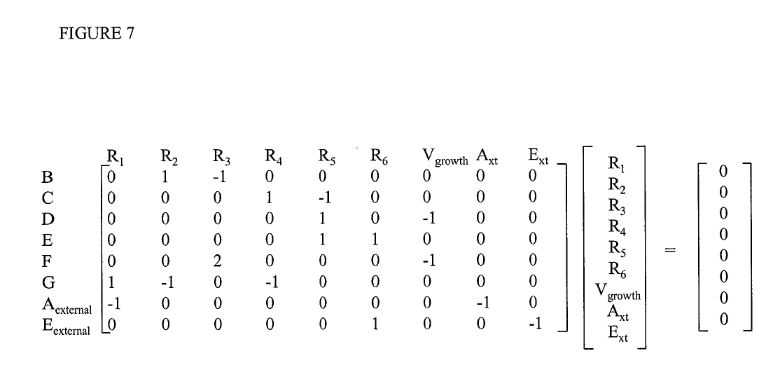

A hypothetical reaction network is provided in FIG. 5 to exemplify the above-described reactions and their interactions. The reactions can be represented in the exemplary data structure shown in FIG. 7 as set forth below. The reaction network, shown in FIG. 5, includes intrasystem reactions that occur entirely within the compartment indicated by the shaded oval such as reversible reaction R₂ which acts on reactants B and G and reaction R₃ which converts one equivalent of B to 2 equivalents of F. The reaction network shown in FIG. 5 also contains exchange reactions such as input/output exchange reactions Aₓₜ and Eₓₜ, and the demand exchange reaction, V_{growth}, which represents growth in response to the one equivalent of D and one equivalent of F. Other intrasystem reactions include R₁ which is a translocation and transformation reaction that translocates reactant A into the compartment and transforms it to reactant G and reaction R₆ which is a transport reaction that translocates reactant E out of the compartment.

A reaction network can be represented as a set of linear algebraic equations which can be presented as a stoichiometric matrix S, with S being an m×n matrix where m corresponds to the number of reactants or metabolites and n corresponds to the number of reactions taking place in the network. An example of a stoichiometric matrix representing the reaction network of FIG. 5 is shown in FIG. 7. As shown in FIG. 7, each column in the matrix corresponds to a particular reaction n, each row corresponds to a particular reactant m, and each Sₘₙ element corresponds to the stoichiometric coefficient of the reactant m in the reaction denoted n. The stoichiometric matrix includes intra-system reactions such as R₂ and R₃ which are related to reactants that participate in the respective reactions according to a stoichiometric coefficient having a sign indicative of whether the reactant is a substrate or product of the reaction and a value correlated with the number of equivalents of the reactant consumed or produced by the reaction. Exchange reactions such as −Eₓₜ and −Aₓₜ are similarly correlated with a stoichiometric coefficient. As exemplified by reactant E, the same compound can be treated separately as an internal reactant (E) and an external reactant (Eₑₓₜₑᵣₙₐₗ) such that an exchange reaction (R₆) exporting the compound is correlated by stoichiometric coefficients of −1 and 1, respectively. However, because the compound is treated as a separate reactant by virtue of its compartmental location, a reaction, such as R₅, which produces the internal reactant (E) but does not act on the external reactant (Eₑₓₜₑᵣₙₐₗ) is correlated by stoichiometric coefficients of 1 and 0, respectively. Demand reactions such as V_{growth} can also be included in the stoichiometric matrix being correlated with substrates by an appropriate stoichiometric coefficient.

As set forth in further detail below, a stoichiometric matrix provides a convenient format for representing and analyzing a reaction network because it can be readily manipulated and used to compute network properties, for example, by using linear programming or general convex analysis. A reaction network data structure can take on a variety of formats so long as it is capable of relating reactants and reactions in the manner exemplified above for a stoichiometric matrix and in a manner that can be manipulated to determine an activity of one or more reactions using methods such as those exemplified below. Other examples of reaction network data structures that are useful in the invention include a connected graph, list of chemical reactions or a table of reaction equations.

A reaction network data structure can be constructed to include all reactions that are involved in Bacillus subtilis metabolism or any portion thereof. A portion of Bacillus subtilis metabolic reactions that can be included in a reaction network data structure of the invention includes, for example, a central metabolic pathway such as glycolysis, the TCA cycle, the PPP or ETS; or a peripheral metabolic pathway such as amino acid biosynthesis, amino acid degradation, purine biosynthesis, pyrimidine biosynthesis, lipid biosynthesis, fatty acid metabolism, vitamin or cofactor biosynthesis, cell wall metabolism, transport processes and alternative carbon source catabolism. Examples of individual pathways within the peripheral pathways are set forth in Table 8, including, for example, the cofactor biosynthesis pathways for isoprenoid biosynthesis, quinone biosynthesis, enterochelin biosynthesis, riboflavin biosynthesis, folate biosyntheis, coenzyme A biosynthesis, NAD biosynthesis, tetrapyrrole biosynthesis, biotin biosynthesis and thaimin biosynthesis. A reaction network can also include the production of a particular protein such as amylase or its secretion or both as demonstrated in the Examples below.

Depending upon a particular application, a reaction network data structure can include a plurality of Bacillus subtilis reactions including any or all of the reactions listed in Table 8. Exemplary reactions that can be included are those that are identified as being required to achieve a desired B. subtilis growth rate or activity including, for example, reactions identified as SUCA, GND, PGL, ACKA, ACS, ACNA, GLTA, ENO, FBP, FBA, FRDA, GLK2, ZWF, GAPA, ICDA, MDH, PC, PFKA, PGI1, PGK, PTA, GPMA, ACEE, PYKF, RPIA, ARAD, SDHA1, TKTA1 or TPIA in Table 7. Other reactions that can be included are those that are not described in the literature or genome annotation but can be identified during the course of iteratively developing a B. subtilis model of the invention including, for example, reactions identified as ADCSASE, MCCOAC, MGCOAH, ARGA, FORAMD, PMDPHT, PATRAN, PCDCL, PCLIG, NADF, ISPB, HMPK, THIK, BISPHDS, DAPC, METF, MTHIPIS, MTHRKN, MENG, NE1PH, NE3UNK, TNSUNK, SERB, CYSG3, CYSG2, PGPA, PLS2, 3MBACP, 2 MBACP, ISBACP, UDPNA4E, GLMM, MMCOAEP, MMCOAMT or PGL in Table 1. Standard chemical names for the acronyms used to identify the reactants in the reactions of Tables 1 and 7 are provided in Table 9.

For some applications, it can be advantageous to use a reaction network data structure that includes a minimal number of reactions to achieve a particular B. subtilis activity under a particular set of environmental conditions. A reaction network data structure having a minimal number of reactions can be identified by performing the simulation methods described below in an iterative fashion where different reactions or sets of reactions are systematically removed and the effects observed. As demonstrated in Example V, such methods were used to identify a reaction network data structure having at least 252 reactions. Accordingly, the invention provides a computer readable medium, containing a data structure relating a plurality of Bacillus subtilis reactants to a plurality of Bacillus subtilis reactions, wherein the plurality of Bacillus subtilis reactions contains at least 252 reactions. In another embodiment, a data structure of the invention can exclude one or more peripheral pathway including, for example, the cofactor biosynthesis pathways for isoprenoid biosynthesis, quinone biosynthesis, enterochelin biosynthesis, riboflavin biosynthesis, folate biosyntheis, coenzyme A biosynthesis, NAD biosynthesis, tetrapyrrole biosynthesis, biotin biosynthesis and thaimin biosynthesis.

Depending upon the particular environmental conditions being tested and the desired activity, a reaction network data structure can contain smaller numbers of reactions such as at least 200, 150, 100 or 50 reactions. A reaction network data structure having relatively few reactions can provide the advantage of reducing computation time and resources required to perform a simulation. When desired, a reaction network data structure having a particular subset of reactions can be made or used in which reactions that are not relevant to the particular simulation are omitted. Alternatively, larger numbers of reactions can be included in order to increase the accuracy or molecular detail of the methods of the invention or to suit a particular application. Thus, a reaction network data structure can contain at least 300, 350, 400, 450, 500, 550, 600 or more reactions up to the number of reactions that occur in or by B. subtilis or that are desired to simulate the activity of the full set of reactions occurring in B. subtilis. A reaction network data structure that is substantially complete with respect to the metabolic reactions of B. subtilis provides the advantage of being relevant to a wide range of conditions to be simulated, whereas those with smaller numbers of metabolic reactions are limited to a particular subset of conditions to be simulated.

A B. subtilis reaction network data structure can include one or more reactions that occur in or by Bacillus subtilis and that do not occur, either naturally or following manipulation, in or by another organism, such as Escherichia coli, Haemophilus influenzae, Saccharomyces cerevisiae or human. Examples of reactions that are unique to B. subtilis compared to Escherichia coli, Haemophilus influenzae, Saccharomyces cerevisiae and human include those identified in Table 8 as any of BS001 through BS125. It is understood that a B. subtilis reaction network data structure can also include one or more reactions that occur in another organism. Addition of such heterologous reactions to a reaction network data structure of the invention can be used in methods to predict the consequences of heterologous gene transfer and protein expression in B. subtilis, for example, when designing or engineering man-made strains.

The reactions included in a reaction network data structure of the invention can be metabolic reactions. A reaction network data structure can also be constructed to include other types of reactions such as regulatory reactions, signal transduction reactions, cell cycle reactions, reactions controlling developmental processes such as sporulation, reactions involved in protein synthesis and regulation thereof, reactions involved in gene transcription and translation, and regulation thereof, and reactions involved in assembly of a cell and its subcellular components.

A reaction network data structure or index of reactions used in the data structure such as that available in a metabolic reaction database, as described above, can be annotated to include information about a particular reaction. A reaction can be annotated to indicate, for example, assignment of the reaction to a protein, macromolecule or enzyme that performs the reaction, assignment of a gene(s) that codes for the protein, macromolecule or enzyme, the Enzyme Commission (EC) number of the particular metabolic reaction, a subset of reactions to which the reaction belongs, citations to references from which information was obtained, or a level of confidence with which a reaction is believed to occur in B. subtilis. A computer readable medium or media of the invention can include a gene database containing annotated reactions. Such information can be obtained during the course of building a metabolic reaction database or model of the invention as described below.

As used herein, the term “gene database” is intended to mean a computer readable medium or media that contains at least one reaction that is annotated to assign a reaction to one or more macromolecules that perform the reaction or to assign one or more nucleic acid that encodes the one or more macromolecules that perform the reaction. A gene database can contain a plurality of reactions some or all of which are annotated. An annotation can include, for example, a name for a macromolecule; assignment of a function to a macromolecule; assignment of an organism that contains the macromolecule or produces the macromolecule; assignment of a subcellular location for the macromolecule; assignment of conditions under which a macromolecule is regulated with respect to performing a reaction, being expressed or being degraded; assignment of a cellular component that regulates a macromolecule; an amino acid or nucleotide sequence for the macromolecule; or any other annotation found for a macromolecule in a genome database such as those that can be found in Genbank, a site maintained by the NCBI (ncbi.nlm.gov) or the Subtilist database (see, for example, Moszer et al., Nucl. Acids Res. 30:62-65 (2002)).

A gene database of the invention can include a substantially complete collection of genes or open reading frames in B. subtilis or a substantially complete collection of the macromolecules encoded by the B. subtilis genome. Alternatively, a gene database can include a portion of genes or open reading frames in B. subtilis or a portion of the macromolecules encoded by the B. subtilis genome. The portion can be at least 10%, 15% 20%, 25%, 50%, 75%, 90% or 95% of the genes or open reading frames encoded by the B. subtilis genome, or the macromolecules encoded therein. A gene database can also include macromolecules encoded by at least a portion of the nucleotide sequence for the B. subtilis genome such as at least 10%, 15%, 20%, 25%, 50%, 75%, 90% or 95% of the B. subtilis genome. Accordingly, a computer readable medium or media of the invention can include at least one reaction for each macromolecule encoded by a portion of the B. subtilis genome.

An in silico B. subtilis model of the invention can be built by an iterative process which includes gathering information regarding particular reactions to be added to a model, representing the reactions in a reaction network data structure, and performing preliminary simulations wherein a set of constraints is placed on the reaction network and the output evaluated to identify errors in the network. Errors in the network such as gaps that lead to non-natural accumulation or consumption of a particular metabolite can be identified as described below and simulations repeated until a desired performance of the model is attained. An exemplary method for iterative model construction is provided in Example I.

Thus, the invention provides a method for making a data structure relating a plurality of Bacillus subtilis reactants to a plurality of Bacillus subtilis reactions in a computer readable medium or media. The method includes the steps of: (a) identifying a plurality of Bacillus subtilis reactions and a plurality of Bacillus subtilis reactants that are substrates and products of the Bacillus subtilis reactions; (b) relating the plurality of Bacillus subtilis reactants to the plurality of Bacillus subtilis reactions in a data structure, wherein each of the Bacillus subtilis reactions includes a reactant identified as a substrate of the reaction, a reactant identified as a product of the reaction and a stoichiometric coefficient relating the substrate and the product; (c) making a constraint set for the plurality of Bacillus subtilis reactions; (d) providing an objective function; (e) determining at least one flux distribution that minimizes or maximizes the objective function when the constraint set is applied to the data structure, and (f) if the at least one flux distribution is not predictive of Bacillus subtilis physiology, then adding a reaction to or deleting a reaction from the data structure and repeating step (e), if the at least one flux distribution is predictive of Bacillus subtilis physiology, then storing the data structure in a computer readable medium or media.

Information to be included in a data structure of the invention can be gathered from a variety of sources including, for example, the scientific literature or an annotated genome sequence of B. subtilis such as the Subtilist database (see, for example, Moszer et al., Nucl. Acids Res. 30:62-65 (2002)). In the course of developing an in silico model of B. subtilis metabolism, the types of data that can be considered include, for example, biochemical information which is information related to the experimental characterization of a chemical reaction, often directly indicating a protein(s) associated with a reaction and the stoichiometry of the reaction or indirectly demonstrating the existence of a reaction occurring within a cellular extract; genetic information which is information related to the experimental identification and genetic characterization of a gene(s) shown to code for a particular protein(s) implicated in carrying out a biochemical event; genomic information which is information related to the identification of an open reading frame and functional assignment, through computational sequence analysis, that is then linked to a protein performing a biochemical event; physiological information which is information related to overall cellular physiology, fitness characteristics, substrate utilization, and phenotyping results, which provide evidence of the assimilation or dissimilation of a compound used to infer the presence of specific biochemical event (in particular translocations); and modeling information which is information generated through the course of simulating activity of B. subtilis using methods such as those described herein which lead to predictions regarding the status of a reaction such as whether or not the reaction is required to fulfill certain demands placed on a metabolic network.

The majority of the reactions occurring in B. subtilis reaction networks are catalyzed by enzymes/proteins, which are created through the transcription and translation of the genes found within the chromosome in the cell. The remaining reactions occur either spontaneously or through non-enzymatic processes. Furthermore, a reaction network data structure can contain reactions that add or delete steps to or from a particular reaction pathway. For example, reactions can be added to optimize or improve performance of a B. subtilis model in view of empirically observed activity. Alternatively, reactions can be deleted to remove intermediate steps in a pathway when the intermediate steps are not necessary to model flux through the pathway. For example, if a pathway contains 3 nonbranched steps, the reactions can be combined or added together to give a net reaction, thereby reducing memory required to store the reaction network data structure and the computational resources required for manipulation of the data structure. An example of a combined reaction is that for UDP-N-acetylglucosamine diphosphorylase shown in Table 8, which combines the reactions for glucosamine-1-phosphate N-acetyltransferase and UDP-N-acetylglucosamine diphosphorylase.

The reactions that occur due to the activity of gene-encoded enzymes can be obtained from a genome database which lists genes identified from genome sequencing and subsequent genome annotation. Genome annotation consists of the locations of open reading frames and assignment of function from homology to other known genes or empirically determined activity. Such a genome database can be acquired through public or private databases containing annotated B. subtilis nucleic acid or protein sequences. If desired, a model developer can perform a network reconstruction and establish the model content associations between the genes, proteins, and reactions as described, for example, in Covert et al. Trends in Biochemical Sciences 26:179-186 (2001) and Palsson, WO 00/46405.

As reactions are added to a reaction network data structure or metabolic reaction database, those having known or putative associations to the proteins/enzymes which enable/catalyze the reaction and the associated genes that code for these proteins can be identified by annotation. Accordingly, the appropriate associations for all of the reactions to their related proteins or genes or both can be assigned. These associations can be used to capture the non-linear relationship between the genes and proteins as well as between proteins and reactions. In some cases one gene codes for one protein which then perform one reaction. However, often there are multiple genes which are required to create an active enzyme complex and often there are multiple reactions that can be carried out by one protein or multiple proteins that can carry out the same reaction. These associations capture the logic (i.e. AND or OR relationships) within the associations. Annotating a metabolic reaction database with these associations can allow the methods to be used to determine the effects of adding or eliminating a particular reaction not only at the reaction level, but at the genetic or protein level in the context of running a simulation or predicting B. subtilis activity.

A reaction network data structure of the invention can be used to determine the activity of one or more reactions in a plurality of B. subtilis reactions independent of any knowledge or annotation of the identity of the protein that performs the reaction or the gene encoding the protein. A model that is annotated with gene or protein identities can include reactions for which a protein or encoding gene is not assigned. While a large portion of the reactions in a cellular metabolic network are associated with genes in the organism's genome, there are also a substantial number of reactions included in a model for which there are no known genetic associations. Such reactions can be added to a reaction database based upon other information that is not necessarily related to genetics such as biochemical or cell based measurements or theoretical considerations based on observed biochemical or cellular activity. For example, there are many reactions that can either occur spontaneously or are not protein-enabled reactions. Furthermore, the occurrence of a particular reaction in a cell for which no associated proteins or genetics have been currently identified can be indicated during the course of model building by the iterative model building methods of the invention.

The reactions in a reaction network data structure or reaction database can be assigned to subsystems by annotation, if desired. The reactions can be subdivided according to biological criteria, such as according to traditionally identified metabolic pathways (glycolysis, amino acid metabolism and the like) or according to mathematical or computational criteria that facilitate manipulation of a model that incorporates or manipulates the reactions. Methods and criteria for subdviding a reaction database are described in further detail in Schilling et al., J. Theor. Biol. 203:249-283 (2000). The use of subsystems can be advantageous for a number of analysis methods, such as extreme pathway analysis, and can make the management of model content easier. Although assigning reactions to subsystems can be achieved without affecting the use of the entire model for simulation, assigning reactions to subsystems can allow a user to search for reactions in a particular subsystem which may be useful in performing various types of analyses. Therefore, a reaction network data structure can include any number of desired subsystems including, for example, 2 or more subsystems, 5 or more subsystems, 10 or more subsystems, 25 or more subsystems or 50 or more subsystems.

The reactions in a reaction network data structure or metabolic reaction database can be annotated with a value indicating the confidence with which the reaction is believed to occur in B. subtilis. The level of confidence can be, for example, a function of the amount and form of supporting data that is available. This data can come in various forms including published literature, documented experimental results, or results of computational analyses. Furthermore, the data can provide direct or indirect evidence for the existence of a chemical reaction in a cell based on genetic, biochemical, and/or physiological data.

The invention further provides a computer readable medium, containing (a) a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating the substrate and the product, and (b) a constraint set for the plurality of Bacillus subtilis reactions.

Constraints can be placed on the value of any of the fluxes in the metabolic network using a constraint set!. These constraints can be representative of a minimum or maximum allowable flux through a given reaction, possibly resulting from a limited amount of an enzyme present. Additionally, the constraints can determine the direction or reversibility of any of the reactions or transport fluxes in the reaction network data structure. Based on the in vivo environment where B. subtilis lives the metabolic resources available to the cell for biosynthesis of essential molecules for can be determined. Allowing the corresponding transport fluxes to be active provides the in silico B. subtilis with inputs and outputs for substrates and by-products produced by the metabolic network.

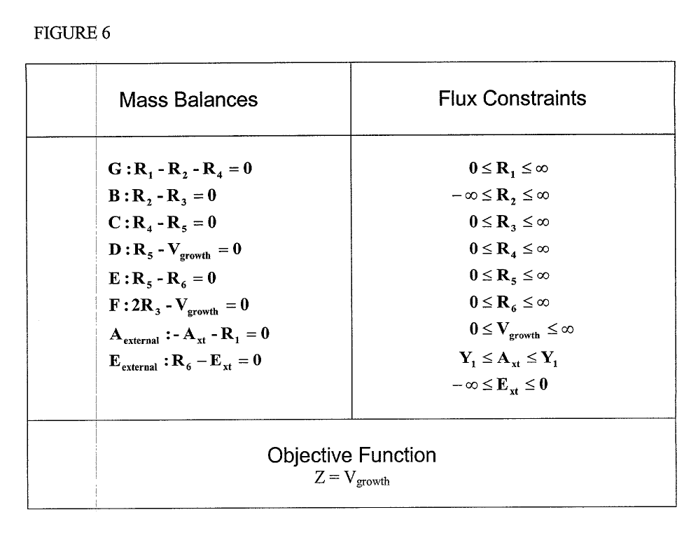

Returning to the hypothetical reaction network shown in FIG. 5, constraints can be placed on each reaction in the exemplary format, shown in FIG. 6, as follows. The constraints are provided in a format that can be used to constrain the reactions of the stoichiometric matrix shown in FIG. 7. The format for the constraints used for a matrix or in linear programming can be conveniently represented as a linear inequality such as

βⱼ≦vⱼ≦αⱼ:j=1 . . . n (Eq. 1)

where vⱼ is the metabolic flux vector, βⱼ is the minimum flux value and αⱼ is the maximum flux value. Thus, αⱼ can take on a finite value representing a maximum allowable flux through a given reaction or βⱼ can take on a finite value representing minimum allowable flux through a given reaction. Additionally, if one chooses to leave certain reversible reactions or transport fluxes to operate in a forward and reverse manner the flux may remain unconstrained by setting βⱼ to negative infinity and αⱼ to positive infinity as shown for reaction R₂ in FIG. 6. If reactions proceed only in the forward reaction βⱼ is set to zero while αⱼ is set to positive infinity as shown for reactions R₁, R₃, R₄, R₅, and R₆ in FIG. 6. As an example, to simulate the event of a genetic deletion or non-expression of a particular protein, the flux through all of the corresponding metabolic reactions related to the gene or protein in question are reduced to zero by setting αⱼ and βⱼ to be zero. Furthermore, if one wishes to simulate the absence of a particular growth substrate one can simply constrain the corresponding transport fluxes that allow the metabolite to enter the cell to be zero by setting αⱼ and βⱼ to be zero. On the other hand if a substrate is only allowed to enter or exit the cell via transport mechanisms, the corresponding fluxes can be properly constrained to reflect this scenario.

The ability of a reaction to be actively occurring is dependent on a large number of additional factors beyond just the availability of substrates. These factors, which can be represented as variable constraints in the models and methods of the invention include, for example, the presence of cofactors necessary to stabilize the protein/enzyme, the presence or absence of enzymatic inhibition and activation factors, the active formation of the protein/enzyme through translation of the corresponding mRNA transcript, the transcription of the associated gene(s) or the presence of chemical signals and/or proteins that assist in controlling these processes that ultimately determine whether a chemical reaction is capable of being carried out within an organism. Regulation can be represented in an in silico B. subtilis model by providing a variable constraint as set forth below.

Thus, the invention provides a computer readable medium or media, including (a) a data structure relating a plurality of B. subtilis reactants to a plurality of B. subtilis reactions, wherein each of the reactions includes a reactant identified as a substrate of the reaction, a reactant identified as a product of the reaction and a stoichiometric coefficient relating the substrate and the product, and wherein at least one of the reactions is a regulated reaction; and (b) a constraint set for the plurality of reactions, wherein the constraint set includes a variable constraint for the regulated reaction.

As used herein, the term “regulated,” when used in reference to a reaction in a data structure, is intended to mean a reaction that experiences an altered flux due to a change in the value of a constraint or a reaction that has a variable constraint.

As used herein, the term “regulatory reaction” is intended to mean a chemical conversion or interaction that alters the activity of a protein, macromolecule or enzyme. A chemical conversion or interaction can directly alter the activity of a protein, macromolecule or enzyme such as occurs when the protein, macromolecule or enzyme is post-translationally modified or can indirectly alter the activity of a protein, macromolecule or enzyme such as occurs when a chemical conversion or binding event leads to altered expression of the protein, macromolecule or enzyme. Thus, transcriptional or translational regulatory pathways can indirectly alter a protein, macromolecule or enzyme or an associated reaction. Similarly, indirect regulatory reactions can include reactions that occur due to downstream components or participants in a regulatory reaction network. When used in reference to a data structure or in silico B. subtilis model, the term is intended to mean a first reaction that is related to a second reaction by a function that alters the flux through the second reaction by changing the value of a constraint on the second reaction.

As used herein, the term “regulatory data structure” is intended to mean a representation of an event, reaction or network of reactions that activate or inhibit a reaction, the representation being in a format that can be manipulated or analyzed. An event that activates a reaction can be an event that initiates the reaction or an event that increases the rate or level of activity for the reaction. An event that inhibits a reaction can be an event that stops the reaction or an event that decreases the rate or level of activity for the reaction. Reactions that can be represented in a regulatory data structure include, for example, reactions that control expression of a macromolecule that in turn, performs a reaction such as transcription and translation reactions, reactions that lead to post translational modification of a protein or enzyme such as phophorylation, dephosphorylation, prenylation, methylation, oxidation or covalent modification, reactions that process a protein or enzyme such as removal of a pre- or pro-sequence, reactions that degrade a protein or enzyme or reactions that lead to assembly of a protein or enzyme.

As used herein, the term “regulatory event” is intended to mean a modifier of the flux through a reaction that is independent of the amount of reactants available to the reaction. A modification included in the term can be a change in the presence, absence, or amount of an enzyme that performs a reaction. A modifier included in the term can be a regulatory reaction such as a signal transduction reaction or an environmental condition such as a change in pH, temperature, redox potential or time. It will be understood that when used in reference to an in silico B. subtilis model or data structure a regulatory event is intended to be a representation of a modifier of the flux through a B. subtilis reaction that is independent of the amount of reactants available to the reaction.

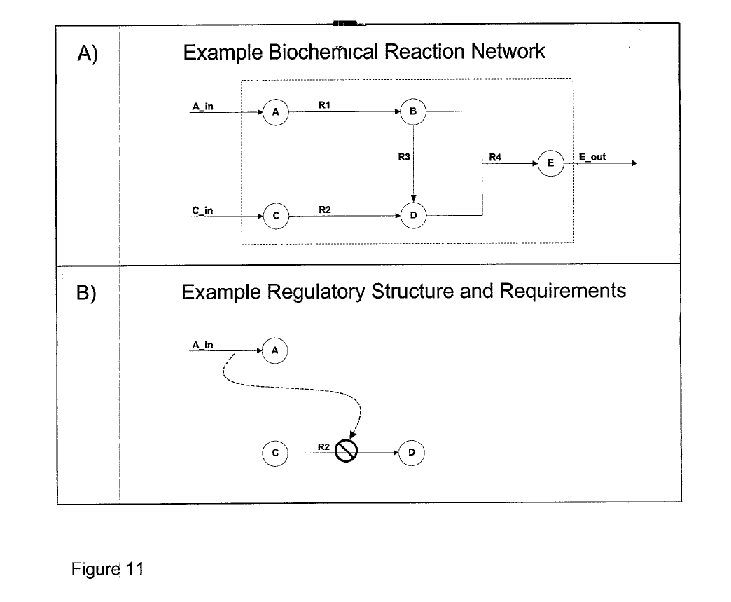

The effects of regulation on one or more reactions that occur in B. subtilis can be predicted using an in silico B. subtilis model of the invention. Regulation can be taken into consideration in the context of a particular condition being examined by providing a variable constraint for the reaction in an in silico B. subtilis model. Such constraints constitute condition-dependent constraints. A data structure can represent regulatory reactions as Boolean logic statements (Reg-reaction). The variable takes on a value of 1 when the reaction is available for use in the reaction network and will take on a value of 0 if the reaction is restrained due to some regulatory feature. A series of Boolean statements can then be introduced to mathematically represent the regulatory network as described for example in Covert et al. J. Theor. Biol. 2131:73-88 (2001). For example, in the case of a transport reaction (A_in) that imports metabolite A, where metabolite A inhibits reaction R2 as shown in FIG. 11, a boolean rule can state that:

Reg−R2=IF NOT(A_in). (Eq. 2)

This statement indicates that reaction R2 can occur if reaction A_in is not occurring (i.e. if metabolite A is not present). Similarly, it is possible to assign the regulation to a variable A which would indicate an amount of A above or below a threshold that leads to the inhibition of reaction R2. Any function that provides values for variables corresponding to each of the reactions in the biochemical reaction network can be used to represent a regulatory reaction or set of regulatory reactions in a regulatory data structure. Such functions can include, for example, fuzzy logic, heuristic rule-based descriptions, differential equations or kinetic equations detailing system dynamics.

A reaction constraint placed on a reaction can be incorporated into an in silico B. subtilis model using the following general equation:

(Reg-Reaction)*βⱼ≦vⱼ≦αⱼ*(Reg-Reaction): j=1 . . . n (Eq. 3)

For the example of reaction R2 this equation is written as follows:

(0)*Reg−R2≦R2≦(∞)*Reg−R2. (Eq. 4)

Thus, during the course of a simulation, depending upon the presence or absence of metabolite A in the interior of the cell where reaction R2 occurs, the value for the upper boundary of flux for reaction R2 will change from 0 to infinity, respectively.

With the effects of a regulatory event or network taken into consideration by a constraint function and the condition-dependent constraints set to an initial relevant value, the behavior of the B. subtilis reaction network can be simulated for the conditions considered as set forth below.

Although regulation has been exemplified above for the case where a variable constraint is dependent upon the outcome of a reaction in the data structure, a plurality of variable constraints can be included in an in silico B. subtilis model to represent regulation of a plurality of reactions. Furthermore, in the exemplary case set forth above, the regulatory structure includes a general control stating that a reaction is inhibited by a particular environmental condition. Using a general control of this type, it is possible to incorporate molecular mechanisms and additional detail into the regulatory structure that is responsible for determining the active nature of a particular chemical reaction within an organism.

Regulation can also be simulated by a model of the invention and used to predict a B. subtilis physiological function without knowledge of the precise molecular mechanisms involved in the reaction network being modeled. Thus, the model can be used to predict, in silico, overall regulatory events or causal relationships that are not apparent from in vivo observation of any one reaction in a network or whose in vivo effects on a particular reaction are not known. Such overall regulatory effects can include those that result from overall environmental conditions such as changes in pH, temperature, redox potential, or the passage of time.

The in silico B. subtilis model and methods described herein can be implemented on any conventional host computer system, such as those based on Intel.RTM. microprocessors and running Microsoft Windows operating systems. Other systems, such as those using the UNIX or LINUX operating system and based on IBM.RTM., DEC.RTM. or Motorola.RTM. microprocessors are also contemplated. The systems and methods described herein can also be implemented to run on client-server systems and wide-area networks, such as the Internet.

Software to implement a method or model of the invention can be written in any well-known computer language, such as Java, C, C++, Visual Basic, FORTRAN or COBOL and compiled using any well-known compatible compiler. The software of the invention normally runs from instructions stored in a memory on a host computer system. A memory or computer readable medium can be a hard disk, floppy disc, compact disc, magneto-optical disc, Random Access Memory, Read Only Memory or Flash Memory. The memory or computer readable medium used in the invention can be contained within a single computer or distributed in a network. A network can be any of a number of conventional network systems known in the art such as a local area network (LAN) or a wide area network (WAN). Client-server environments, database servers and networks that can be used in the invention are well known in the art. For example, the database server can run on an operating system such as UNIX, running a relational database management system, a World Wide Web application and a World Wide Web server. Other types of memories and computer readable media are also contemplated to function within the scope of the invention.

A database or data structure of the invention can be represented in a markup language format including, for example, Standard Generalized Markup Language (SGML), Hypertext markup language (HTML) or Extensible Markup language (XML). Markup languages can be used to tag the information stored in a database or data structure of the invention, thereby providing convenient annotation and transfer of data between databases and data structures. In particular, an XML format can be useful for structuring the data representation of reactions, reactants and their annotations; for exchanging database contents, for example, over a network or internet; for updating individual elements using the document object model; or for providing differential access to multiple users for different information content of a data base or data structure of the invention. XML programming methods and editors for writing XML code are known in the art as described, for example, in Ray, “Learning XML” O'Reilly and Associates, Sebastopol, Calif. (2001).

A set of constraints can be applied to a reaction network data structure to simulate the flux of mass through the reaction network under a particular set of environmental conditions specified by a constraints set. Because the time constants characterizing metabolic transients and/or metabolic reactions are typically very rapid, on the order of milli-seconds to seconds, compared to the time constants of cell growth on the order of hours to days, the transient mass balances can be simplified to only consider the steady state behavior. Referring now to an example where the reaction network data structure is a stoichiometric matrix, the steady state mass balances can be applied using the following system of linear equations

S·v=0 (Eq. 5)

where S is the stoichiometric matrix as defined above and v is the flux vector. This equation defines the mass, energy, and redox potential constraints placed on the metabolic network as a result of stoichiometry. Together Equations 1 and 5 representing the reaction constraints and mass balances, respectively, effectively define the capabilities and constraints of the metabolic genotype and the organism's metabolic potential. All vectors, v, that satisfy Equation 5 are said to occur in the mathematical nullspace of S. Thus, the null space defines steady-state metabolic flux distributions that do not violate the mass, energy, or redox balance constraints. Typically, the number of fluxes is greater than the number of mass balance constraints, thus a plurality of flux distributions satisfy the mass balance constraints and occupy the null space. The null space, which defines the feasible set of metabolic flux distributions, is further reduced in size by applying the reaction constraints set forth in Equation 1 leading to a defined solution space. A point in this space represents a flux distribution and hence a metabolic phenotype for the network. An optimal solution within the set of all solutions can be determined using mathematical optimization methods when provided with a stated objective and a constraint set. The calculation of any solution constitutes a simulation of the model.

Objectives for activity of B. subtilis can be chosen to explore the improved use of the metabolic network within a given reaction network data structure. These objectives can be design objectives for a strain, exploitation of the metabolic capabilities of a genotype, or physiologically meaningful objective functions, such as maximum cellular growth. Growth can be defined in terms of biosynthetic requirements based on literature values of biomass composition or experimentally determined values such as those obtained as described above. Thus, biomass generation can be defined as an exchange reaction that removes intermediate metabolites in the appropriate ratios and represented as an objective function. In addition to draining intermediate metabolites this reaction flux can be formed to utilize energy molecules such as ATP, NADH and NADPH so as to incorporate any maintenance requirement that must be met. This new reaction flux then becomes another constraint/balance equation that the system must satisfy as the objective function. Using the stoichiometric matrix of FIG. 7 as an example, adding such a constraint is analogous to adding the additional column V_{growth} to the stoichiometric matrix to represent fluxes to describe the production demands placed on the metabolic system. Setting this new flux as the objective function and asking the system to maximize the value of this flux for a given set of constraints on all the other fluxes is then a method to simulate the growth of the organism.

Continuing with the example of the stoichiometric matrix applying a constraint set to a reaction network data structure can be illustrated as follows. The solution to equation 5 can be formulated as an optimization problem, in which the flux distribution that minimizes a particular objective is found. Mathematically, this optimization problem can be stated as:

Minimize Z (Eq. 6)

where z=Σcᵢ·vᵢ (Eq. 7)

where Z is the objective which is represented as a linear combination of metabolic fluxes vᵢ using the weights cᵢ in this linear combination. The optimization problem can also be stated as the equivalent maximization problem; i.e. by changing the sign on Z. Any commands for solving the optimazation problem can be used including, for example, linear programming commands.

A computer system of the invention can further include a user interface capable of receiving a representation of one or more reactions. A user interface of the invention can also be capable of sending at least one command for modifying the data structure, the constraint set or the commands for applying the constraint set to the data representation, or a combination thereof. The interface can be a graphic user interface having graphical means for making selections such as menus or dialog boxes. The interface can be arranged with layered screens accessible by making selections from a main screen. The user interface can provide access to other databases useful in the invention such as a metabolic reaction database or links to other databases having information relevant to the reactions or reactants in the reaction network data structure or to B. subtilis physiology. Also, the user interface can display a graphical representation of a reaction network or the results of a simulation using a model of the invention.

Once an initial reaction network data structure and set of constraints has been created, this model can be tested by preliminary simulation. During preliminary simulation, gaps in the network or “dead-ends” in which a metabolite can be produced but not consumed or where a metabolite can be consumed but not produced can be identified. Based on the results of preliminary simulations areas of the metabolic reconstruction that require an additional reaction can be identified. The determination of these gaps can be readily calculated through appropriate queries of the reaction network data structure and need not require the use of simulation strategies, however, simulation would be an alternative approach to locating such gaps.

In the preliminary simulation testing and model content refinement stage the existing model is subjected to a series of functional tests to determine if it can perform basic requirements such as the ability to produce the required biomass constituents and generate predictions concerning the basic physiological characteristics of the particular organism strain being modeled. The more preliminary testing that is conducted the higher the quality of the model that will be generated. Typically the majority of the simulations used in this stage of development will be single optimizations. A single optimization can be used to calculate a single flux distribution demonstrating how metabolic resources are routed determined from the solution to one optimization problem. An optimization problem can be solved using linear programming as demonstrated in the Examples below. The result can be viewed as a display of a flux distribution on a reaction map. Temporary reactions can be added to the network to determine if they should be included into the model based on modeling/simulation requirements.

Once a model of the invention is sufficiently complete with respect to the content of the reaction network data structure according to the criteria set forth above, the model can be used to simulate activity of one or more reactions in a reaction network. The results of a simulation can be displayed in a variety of formats including, for example, a table, graph, reaction network, flux distribution map or a phenotypic phase plane graph.

Thus, the invention provides a method for predicting a Bacillus subtilis physiological function. The method includes the steps of (a) providing a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating said substrate and said product; (b) providing a constraint set for the plurality of Bacillus subtilis reactions; (c) providing an objective function, and (d) determining at least one flux distribution that minimizes or maximizes the objective function when the constraint set is applied to the data structure, thereby predicting a Bacillus subtilis physiological function.

A method for predicting a Bacillus subtilis physiological function can include the steps of (a) providing a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating the substrate and the product, and wherein at least one of the reactions is a regulated reaction; (b) providing a constraint set for the plurality of reactions, wherein the constraint set includes a variable constraint for the regulated reaction; (c) providing a condition-dependent value to the variable constraint; (d) providing an objective function, and (e) determining at least one flux distribution that minimizes or maximizes the objective function when the constraint set is applied to the data structure, thereby predicting a Bacillus subtilis physiological function.

As used herein, the term “physiological function,” when used in reference to Bacillus subtilis, is intended to mean an activity of a Bacillus subtilis cell as a whole. An activity included in the term can be the magnitude or rate of a change from an initial state of a Bacillus subtilis cell to a final state of the Bacillus subtilis cell. An activity included in the term can be, for example, growth, energy production, redox equivalent production, biomass production, development, or consumption of carbon nitrogen, sulfur, phosphate, hydrogen or oxygen. An activity can also be an output of a particular reaction that is determined or predicted in the context of substantially all of the reactions that affect the particular reaction in a B. subtilis cell or substantially all of the reactions that occur in a B. subtilis cell. Examples of a particular reaction included in the term are production of biomass precursors, production of a protein, production of an amino acid, production of a purine, production of a pyrimidine, production of a lipid, production of a fatty acid, production of a cofactor, production of a cell wall component or transport of a metabolite. A physiological function can include an emergent property which emerges from the whole but not from the sum of parts where the parts are observed in isolation (see for example, Palsson Nat. Biotech 18:1147-1150 (2000)).

A physiological function of B. subtilis reactions can be determined using phase plane analysis of flux distributions. Phase planes are representations of the feasible set which can be presented in two or three dimensions. As an example, two parameters that describe the growth conditions such as substrate and oxygen uptake rates can be defined as two axes of a two-dimensional space. The optimal flux distribution can be calculated from a reaction network data structure and a set of constraints as set forth above for all points in this plane by repeatedly solving the linear programming problem while adjusting the exchange fluxes defining the two-dimensional space. A finite number of qualitatively different metabolic pathway utilization patterns can be identified in such a plane, and lines can be drawn to demarcate these regions. The demarcations defining the regions can be determined using shadow prices of linear optimization as described, for example in Chvatal, Linear Programming New York, W. H. Freeman and Co. (1983). The regions are referred to as regions of constant shadow price structure. The shadow prices define the intrinsic value of each reactant toward the objective function as a number that is either negative, zero, or positive and are graphed according to the uptake rates represented by the x and y axes. When the shadow prices become zero as the value of the uptake rates are changed there is a qualitative shift in the optimal reaction network.

One demarcation line in the phenotype phase plane is defined as the line of optimality (LO). This line represents the optimal relation between respective metabolic fluxes. The LO can be identified by varying the x-axis flux and calculating the optimal y-axis flux with the objective function defined as the growth flux From the phenotype phase plane analysis the conditions under which a desired activity is optimal can be determined. The maximal uptake rates lead to the definition of a finite area of the plot that is the predicted outcome of a reaction network within the environmental conditions represented by the constraint set. Similar analyses can be performed in multiple dimensions where each dimension on the plot corresponds to a different uptake rate. These and other methods for using phase plane analysis, such as those described in Edwards et al., Biotech Bioeng. 77:27-36(2002), can be used to analyze the results of a simulation using an in silico B. subtilis model of the invention.

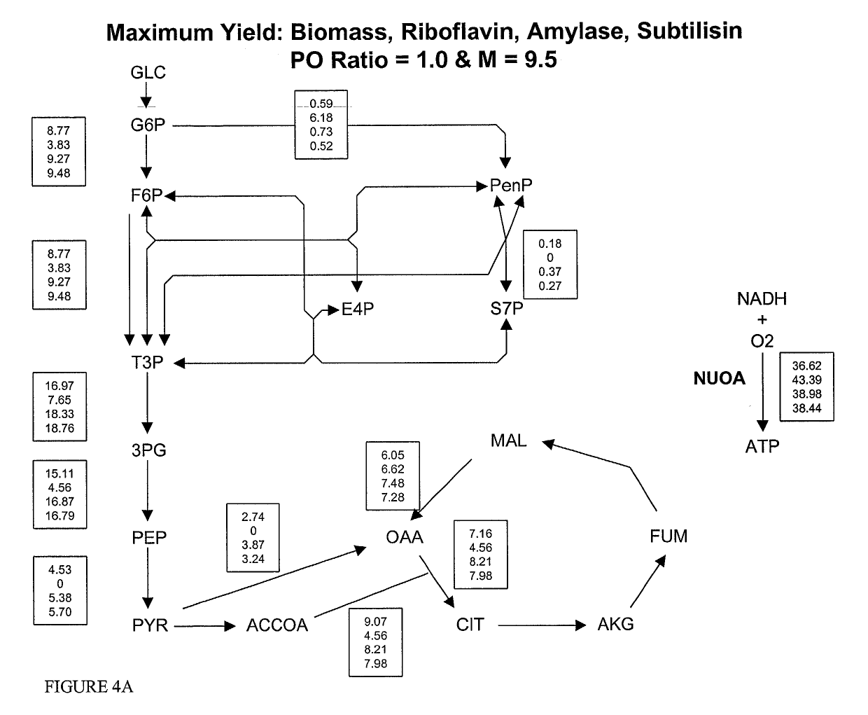

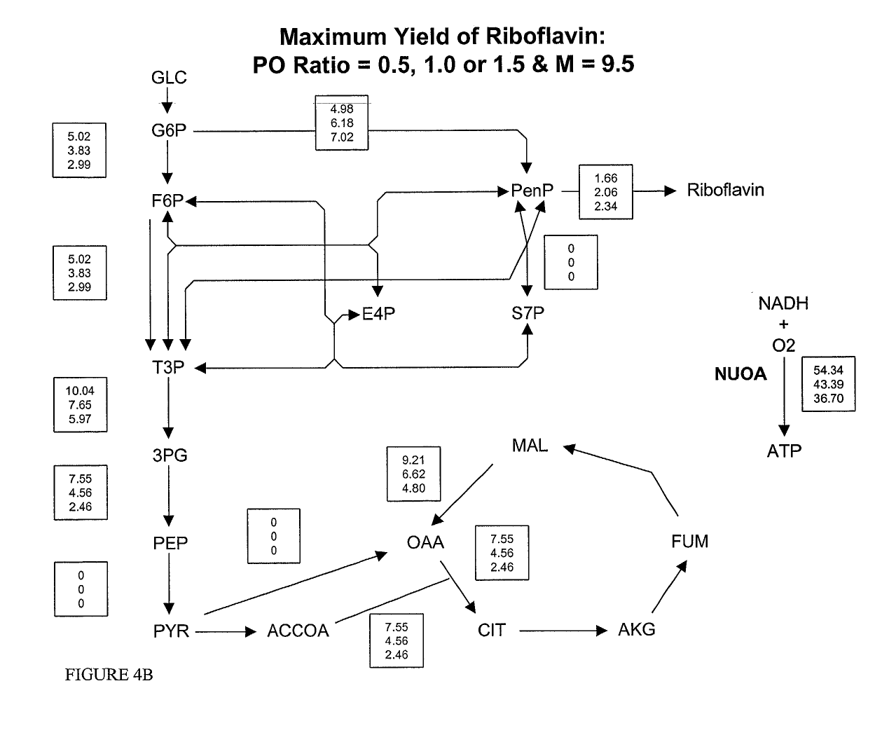

A physiological function of B. subtilis can also be determined using a reaction map to display a flux distribution. A reaction map of B. subtilis can be used to view reaction networks at a variety of levels. In the case of a cellular metabolic reaction network a reaction map can contain the entire reaction complement representing a global perspective. Alternatively, a reaction map can focus on a particular region of metabolism such as a region corresponding to a reaction subsystem described above or even on an individual pathway or reaction. An example of a reaction map showing a subset of reactions in a reaction network of B. subtilis is shown in FIG. 4.

Thus, the invention provides an apparatus that produces a representation of a Bacillus subtilis physiological function, wherein the representation is produced by a process including the steps of: (a) providing a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating said substrate and said product; (b) providing a constraint set for the plurality of Bacillus subtilis reactions; (c) providing an objective function; (d) determining at least one flux distribution that minimizes or maximizes the objective function when the constraint set is applied to the data structure, thereby predicting a Bacillus subtilis physiological function, and (e) producing a representation of the activity of the one or more Bacillus subtilis reactions.

The methods of the invention can be used to determine the activity of a plurality of Bacillus subtilis reactions including, for example, biosynthesis of an amino acid, degradation of an amino acid, biosynthesis of a purine, biosynthesis of a pyrimidine, biosynthesis of a lipid, metabolism of a fatty acid, biosynthesis of a cofactor, metabolism of a cell wall component, transport of a metabolite and metabolism of an alternative carbon source. In addition, the methods can be used to determine the activity of one or more of the reactions described above or listed in Table 8.

The methods of the invention can be used to determine a phenotype of a Bacillus subtilis mutant. The activity of one or more Bacillus subtilis reactions can be determined using the methods described above, wherein the reaction network data structure lacks one or more gene-associated reactions that occur in Bacillus subtilis. Alternatively, the methods can be used to determine the activity of one or more Bacillus subtilis reactions when a reaction that does not naturally occur in B. subtilis is added to the reaction network data structure. Deletion of a gene can also be represented in a model of the invention by constraining the flux through the reaction to zero, thereby allowing the reaction to remain within the data structure. Thus, simulations can be made to predict the effects of adding or removing genes to or from B. subtilis. The methods can be particularly useful for determining the effects of adding or deleting a gene that encodes for a gene product that performs a reaction in a peripheral metabolic pathway.

A drug target or target for any other agent that affects B. subtilis function can be predicted using the methods of the invention. Such predictions can be made by removing a reaction to simulate total inhibition or prevention by a drug or agent. Alternatively, partial inhibition or reduction in the activity a particular reaction can be predicted by performing the methods with altered constraints. For example, reduced activity can be introduced into a model of the invention by altering the αⱼ or βⱼ values for the metabolic flux vector of a target reaction to reflect a finite maximum or minimum flux value corresponding to the level of inhibition. Similarly, the effects of activating a reaction, by initiating or increasing the activity of the reaction, can be predicted by performing the methods with a reaction network data structure lacking a particular reaction or by altering the αⱼ or βⱼ values for the metabolic flux vector of a target reaction to reflect a maximum or minimum flux value corresponding to the level of activation. The methods can be particularly useful for identifying a target in a peripheral metabolic pathway.

Once a reaction has been identified for which activation or inhibition produces a desired effect on B. subtilis function, an enzyme or macromolecule that performs the reaction in B. subtilis or a gene that expresses the enzyme or macromolecule can be identified as a target for a drug or other agent. A candidate compound for a target identified by the methods of the invention can be isolated or synthesized using known methods. Such methods for isolating or synthesizing compounds can include, for example, rational design based on known properties of the target (see, for example, DeCamp et al., Protein Engineering Principles and Practice, Ed. Cleland and Craik, Wiley-Liss, New York, pp. 467-506 (1996)), screening the target against combinatorial libraries of compounds (see for example, Houghten et al., Nature, 354, 84-86 (1991); Dooley et al., Science, 266, 2019-2022 (1994), which describe an iterative approach, or R. Houghten et al. PCT/US91/08694 and U.S. Pat. No. 5,556,762 which describe the positional-scanning approach), or a combination of both to obtain focused libraries. Those skilled in the art will know or will be able to routinely determine assay conditions to be used in a screen based on properties of the target or activity assays known in the art.

A candidate drug or agent, whether identified by the methods described above or by other methods known in the art, can be validated using an in silico B. subtilis model or method of the invention. The effect of a candidate drug or agent on B. subtilis physiological function can be predicted based on the activity for a target in the presence of the candidate drug or agent measured in vitro or in vivo. This activity can be represented in an in silico B. subtilis model by adding a reaction to the model, removing a reaction from the model or adjusting a constraint for a reaction in the model to reflect the measured effect of the candidate drug or agent on the activity of the reaction. By running a simulation under these conditions the holistic effect of the candidate drug or agent on B. subtilis physiological function can be predicted.

The methods of the invention can be used to determine the effects of one or more environmental components or conditions on an activity of Bacillus subtilis. As set forth above an exchange reaction can be added to a reaction network data structure corresponding to uptake of an environmental component, release of a component to the environment, or other environmental demand. The effect of the environmental component or condition can be further investigated by running simulations with adjusted αⱼ or βⱼ values for the metabolic flux vector of the exchange reaction target reaction to reflect a finite maximum or minimum flux value corresponding to the effect of the environmental component or condition. The environmental component can be, for example an alternative carbon source or a metabolite that when added to the environment of B. subtilis can be taken up and metabolized. The environmental component can also be a combination of components present for example in a minimal medium composition. Thus, the methods can be used to determine an optimal or minimal medium composition that is capable of supporting a particular activity of B. subtilis.

The invention further provides a method for determining a set of environmental components to achieve a desired activity for Bacillus subtilis. The method includes the steps of (a) providing a data structure relating a plurality of Bacillus subtilis reactants to a plurality of Bacillus subtilis reactions, wherein each of the Bacillus subtilis reactions includes a reactant identified as a substrate of the reaction, a reactant identified as a product of the reaction and a stoichiometric coefficient relating the substrate and the product; (b)providing a constraint set for the plurality of Bacillus subtilis reactions; (c) applying the constraint set to the data representation, thereby determining the activity of one or more Bacillus subtilis reactions (d) determining the activity of one or more Bacillus subtilis reactions according to steps (a) through (c), wherein the constraint set includes an upper or lower bound on the amount of an environmental component and (e) repeating steps (a) through (c) with a changed constraint set, wherein the activity determined in step (e) is improved compared to the activity determined in step (d).

The following examples are intended to illustrate but not limit the present invention.

### EXAMPLE I

This example shows the construction of a substantially complete B. subtilis metabolic model. This example also demonstrates the iterative model building approach for identifying B. subtilis metabolic reactions that are not present in the scientific literature or genome annotations and adding these reactions to a B. subtilis in silico model to improve the range of physiological functions that can be predicted by the model.

A metabolic reaction database was constructed as follows. The metabolic reactions initially included in the metabolic reaction database were compiled from the biochemical literature (Sonenshein et al., Bacillus subtilis and other gram-positive bacteria: biochemistry, physiology, and molecular genetics. ASM Press, Washington, D.C. (1993) and Sonenshein et al., Bacillus subtilis and its closest relatives: from genes to cells. ASM Press, Washington, D.C. (2002), from genomic reference databases, including SubtiList (described in Moszer et al., Nucleic Acids Res. 30:62-65 and from Kunst et al., Nature 390:249-256 (1997).

Additional reactions, not described in the biochemical literature or genome annotation, were subsequently included in the database following preliminary simulation testing and model content refinement. A list of reactions that were not present in the literature or genome annotations but were determined in the course of metabolic model building to be essential to support growth, as defined by the production of required biomass components, of B. subtilis under several different fermentation conditions is provided in Table 1.

TABLE 1

| | | Reaction |
|---|---|---|
| Enzyme Name | Reaction Stoichiometry | Name |
| Adenosyl | HCYS + ADN <-> SAH | ADCSASE |
| homocysteinase | | |
| (unknown) | | |
| Methylcrotonoyl- | 3M2ECOA + ATP + CO2 -> | MCCOAC |
| CoA carboxylase | 3MGCOA + PI + ADP | |
| Methylgutaconyl- | 3MGCOA -> 3HMGCOA | MGCOAH |
| CoA hydratase | | |
| Formamidase | FAM -> NH3 + FOR | FORAMD |
| Pyrimidine | A6RP5P2 -> A6RP + PI | PMDPHT |
| phosphatase | | |
| Phospho- | 4PPNTE + ATP -> PPI + | PATRAN |
| pantethiene | DPCOA | |
| adenyly- | | |
| transferase | | |
| Phosphopanto- | 4PPNCYS -> CO2 + 4PPNTE | PCDCL |
| thenate-cysteine | | |
| decarboxylase | | |
| Phosphopanto- | 4PPNTO + CTP + CYS -> | PCLIG |
| thenate-cysteine | CMP + PPI + 4PPNCYS | |
| ligase | | |
| NAD kinase | NAD + ATP -> NADP + ADP | NADF |
| Octoprenyl | 5 IPPP + FPP -> OPP + 5 | ISPB |
| pyrophosphate | PPI | |
| synthase | | |
| (5 reactions) | | |
| HMP kinase | AHM + ADP -> AHMP + ADP | HMPK |
| Thiamin kinase | THMP + ADP <-> THIAMIN + | THIK |
| | ATP | |
| 3′-5′ | PAP -> AMP + PI | BISPHDS |
| Bisphosphate | | |
| nucleotidase | | |
| Succinyl | NS2A6O + GLU <-> AKG + | DAPC |
| diaminopimelate | NS26DP | |
| aminotransferase | | |
| Methylene | METTHF + NADH -> NAD + | METF |
| tetrahydrofolate | MTHF | |
| reductase | | |
| 5- | 5MTRP <-> 5MTR1P | MTHIPIS |
| Methylthioribose- | | |
| 1-phosphate | | |
| isomerase | | |
| 5- | 5MTR + ATP -> 5MTRP + | MTHRKN |
| Methylthioribose | ADP | |
| kinase | | |
| S- | DMK + SAM -> MK + SAH | MENG |
| Adenosylmethionine- | | |
| 2-DMK | | |
| methyltransferase | | |
| E-1 (Enolase- | 5MTR1P -> DKMPP | NE1PH |
| phosphatase) | | |
| E-3 (Unknown) | DKMPP -> FOR + KMB | NE3UNK |
| Transamination | KMB + GLN -> GLU + MET | TNSUNK |
| (Unknown) | | |
| Phosphoserine | 3PSER -> PI + SER | SERB |
| phosphatase | | |
| Siroheme | SHCL -> SHEME | CYSG3 |
| ferrochelatase | | |
| 1,3-Dimethyluro- | PC2 + NAD -> NADH + SHCL | CYSG2 |
| (porphyrinogen) | | |
| III dehydrogenase | | |
| Phosphatidyl- | PGP -> PI + PG | PGPA |
| glycerol | | |
| phosphate | | |
| phosphatase A | | |
| Acyltransferase | GL3P + 0.035 | PLS2 |
| | C140ACP + 0.102 C141ACP + | |
| | 0.717 C160ACP + 0.142 | |
| | C161ACP + 1.004 C181ACP -> | |
| | 2ACP + PA | |
| Isovaleryl-CoA | 3MBACP + COA <-> | 3MBACP |
| ACP transacylase | 2MBCOA + ACP | |
| 2-Methylbutyryl- | 2MBACP + COA <-> | 2MBACP |
| CoA ACP | 2MBCOA + ACP | |
| transacylase | | |
| Isobutyryl-CoA | ISBACP + COA <-> | ISBACP |
| ACP transacylase | ISBCOA + ACP | |
| UDP-N- | UDPNAG -> UDPNAGAL | UDPNA4E |
| acetylglucosamine | | |
| 4-epimerase | | |
| Phosphogluco- | GA6P <-> GA1P | GLMM |
| samine mutase | | |
| Methylmalonyl-CoA | SMMCOA <-> RMMCOA | MMCOAEP |
| epimerase | | |
| (5.1.99.1) | | |
| Methylmalonyl-CoA | RMMCOA -> SUCCOA | MMCOAMT |
| mutase (5.4.99.2) | | |
| 6-Phosphoglucono- | D6PGL -> D6PGC | PGL |
| lactonase | | |

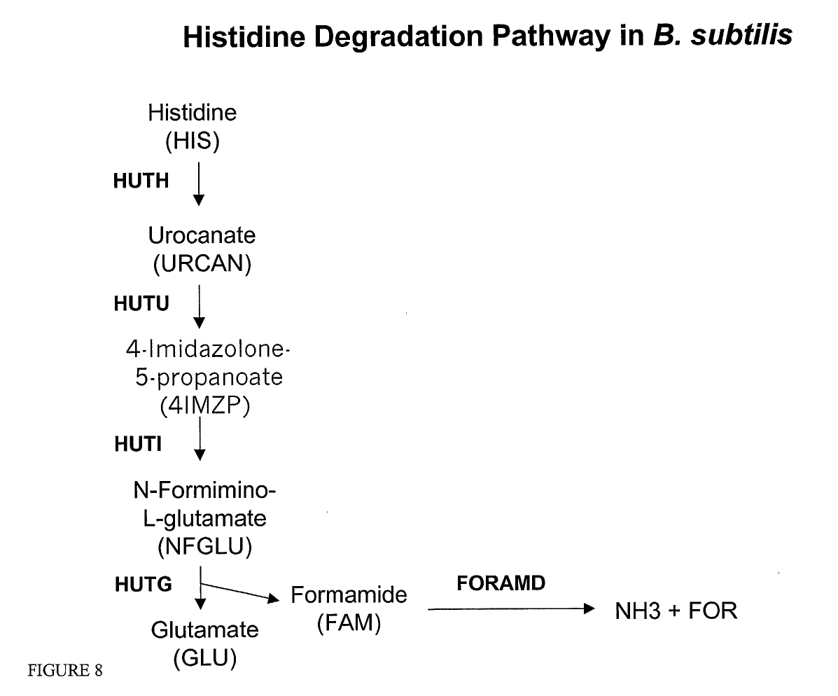

As an example, the formamidase reaction, identified as FORAMD in Table 1, was added to the B. subtilis in silico model as follows. It is known from microbiological experiments that histidine can be metabolized as a carbon and nitrogen source in B. subtilis, indicating that a histidine degradation pathway must be present in the metabolic network (Fisher et al., Bacillus subtilis and its closest relatives: from genes to cells, ASM Press, Washington, D.C. (2002)). Four genes capable of degrading histidine were found in the Subtilist genome sequence and annotation including HUTH, HUTU, HUTI and HUTG. Therefore, to incorporate histidine utilization into the model, the HUTH, HUTU, HUTI and HUTG reactions were added to the stoichiometric matrix and metabolic reaction database to represent the pathway shown in FIG. 8.

A preliminary simulation was run using the stoichiometric matrix having equations for the reactions described in the biochemical literature or genome annotation including HUTH, HUTU, HUTI and HUTG. The simulation was setup with histidine as the only carbon source available to the model by constraining the input/output exchange flux on all other carbon sources to be only positive, whereby only allowing those other compounds to exit the metabolic network. The result of this simulation was that the model could not utilize histidine contrary to experimental evidence. The simulation indicates that the histidine cannot be utilized because the production of formamide (FAM) by the HUTG reaction was found to be unbalanced in the simulation and resulted in a flux of zero for the histidine degradation pathway. There are no reactions in the network capable of using FAM as a substrate to balance the production of FAM by the HUTG reaction. In order to allow the model to represent histidine utilization, a decision was made to balance the production of FAM by adding a reaction that would allow FAM to be utilized by the reaction network. This decision lead to the inclusion of the FORAMD reaction into the network. The simulation was then rerun with this reaction added to the reaction index and hence to the stoichiometric matrix. Addition of the reaction for FORAMD to the stoichiometric matrix was found to balance the production of FAM and to allow flux of mass from histidine through ammonia (NH3) and formate (FOR) to other reactions in the network, thereby simulating histidine utilization as a carbon source for the network in agreement with the true physiology of the organism.

The reactions for methylcrotonoyl-CoA carboxylase (MCCOAC), methylglutaconyl-CoA hydratase (MGCOAH), methylmalonyl-CoA epimerase (MMCOAEP), and methylmalonyl-CoA mutase (MMCOAMT) were also added to the B. subtilis stoichiometric matrix and metabolic reaction database based on iterative model building. The MCCOAC, MGCOAH, MMCOAEP, and MMCOAMT reactions were not apparent from the B. subtilis biochemical literature or from the Subtilist database annotation. However, it is known from microbiological experiments that leucine, isoleucine, and valine are degraded by B. subtilis (Fisher et al., supra (2002)). Therefore, reactions for methylcrotonoyl-CoA carboxylase, methylglutaconyl-CoA hydratase, methylmalonyl-CoA epimerase, and methylmalonyl-CoA mutase were added to complete the degradation pathways. Prior to addition of these reactions the model was not able to accurately predict utilization of leucine, isoleucine, or valine by B. subtilis. However, once the MCCOAC, MGCOAH, MMCOAEP, and MMCOAMT reactions were added, utilization of leucine, isoleucine, or valine by B. subtilis was accurately predicted by the model.

Other reactions added to the metabolic reaction index and stoichiometric matrix during the course of iterative model building included, (1) the pyrimidine phosphatase reaction which, when added, balanced the riboflavin biosynthetic pathway and (2) Isovaleryl-CoA ACP transacylase, 2-methylbutyryl-CoA ACP transacylase and isobutyryl-CoA ACP transacylase which, when added, balanced the production of the multitude of fatty acid structures found in the B. subtilis membranes.

The enzyme 6-phosphogluconolactonase (EC: 3.1.3.31 denoted as PGL) is missing from the Subtilist database. This reaction may or may not be essential for cell growth depending on how constraints are set for the reactions involved in the pentose phosphate pathway. For example, if the transaldolase and transketolase reactions are assumed to be reversible, the cell can replenish all of pentose phosphate intermediates without the action of this enzyme. However, if the transaldolase and transketolase reactions are assumed to be irreversible and operate only in the direction from ribulose 5-phosphate to ribose 5-phosphate and xylulose 5-phosphate, then the PGL reaction becomes essential. Since the latter is most likely operative in most cellular systems, the PGL reaction was added to the reaction database and stoichiometric matrix.

The presence of the PGL reaction in the B. subtilis reaction network was further supported by the results shown in Example V. It was shown by both in silico simulation and in vivo experimentation that deletion of the PGI or GPM reaction was not lethal but only growth-retarding as the pentose phosphate pathway can compensate partially for the inactivity of the glycolytic functions. However, if the PGL reaction is removed from the metabolic network, no carbon flow via PPP can occur which results in no cell growth. Thus, the PGL reaction must be present to reconcile the results of preliminary simulation without the PGL reaction with the results of Example V.

When the 6-phosphoglucolactonase gene of Neisseria meningitides MC58 (nme: NMB1391) was used in a BLAST search of the Bacillus subtilis genome, significant homology (E value of 6E-5) was found with the gamA gene. The gamA gene is putatively assigned to be glucosamine-6-phosphate isomerase (EC: 3.5.99.6). These results demonstrate that a Bacillus subtilis in silico model can be used to identify a putative activity for Bacillus subtilis which can be further used in combination with sequence comparison methods to determine a putative activity for a protein encoded by the Bacillus subtilis genome.

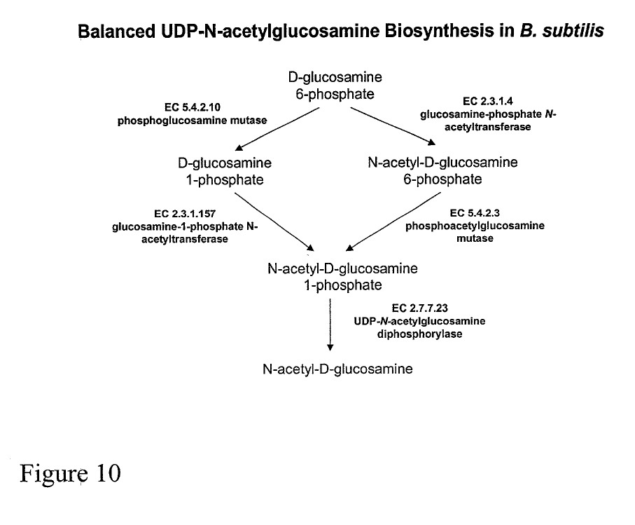

The enzyme phosphoglucosamine mutase (EC:5.4.2.10) is also missing from the Subtilist database. This enzyme is involved in the pathway for bacterial cell-wall peptidoglycan and lipopolysaccharide biosynthesis in E. coli, being an essential step in the pathway for UDP-N-acetylglucosamine biosynthesis. In B. subtilis, UDP-N-acetylglucosamine is required in the synthesis of glycerol techoic acid, a major cell-wall component. The first step in the glycerol techoic acid is catalyzed by the TagO gene product which links the carrier undecaprenyl phosphate with UDP-N-acetylglucosamine to form undecaprenylpyrophosphate-N-acetylglucosamine. FIG. 10 shows two possible routes for the synthesis of UDP-N-acetylglucosamine in E. coli. Neither of these two pathways is complete in the B. subtilis genome but it is likely that one or both of these two pathways is active in B. subtilis.

When the E. coli glmM gene, encoding phosphoglucosamine mutase (EC:5.4.2.10), was searched using BLAST against the Bacillus subtilis genome, two likely candidates were identified: ybbT (E value 1.6e-67) and yhxB (E value 1.2e-7). Annotation for the ybbT gene indicated that its role was “unknown; similar to phosphoglucomutase (EC 5.4.2.2 involved in glycolysis, different from phosphoglucosamine mutase)”. The role of the yhxB was “unknown; similar to phosphomannomutase.” It is therefore very likely that the ybbT gene encodes phosphoglucosamine mutase, and thus the reaction was added to the reaction database and stoichiometric matrix. The pathway from D-glucosamine 6-phosphate to D-glucosaminel-phosphate to N-acetyl-D-glucosaminel-phosphate to N-acetyl-D-glucosamine was chosen to be active in the B. subtilis model.

The alternative route via glucosamine-phosphate N-acetyltransferase was not included since no significant homology was found when the glucosamine-phosphate N-acetyltransferase genes from Drosophila melanogaster and Caenorhabditis elegans were searched using BLAST against the B. subtilis genome.

It should be noted that in Table 8, UDP-N-acetylglucosamine diphosphorylase reaction is combined to catalyze both glucosamine-1-phosphate N-acetyltransferase (EC 2.3.1.157) and UDP-N-acetylglucosamine diphosphorylase reaction.

TABLE 2

| | | | Growth on |
|---|---|---|---|
| | | Putative | glucose of |
| | Reaction | gene | Knockout |
| Enzyme Name | Stoichiometry | assigned | mutant |
| Cardiolipin | 2PG <-> CL + GL | ywnE | Same |
| synthase | | | |
| Tetrahydro- | PIP26DX + SUCCOA -> | ykuQ | |
| dipicolinate | COA + NS2A6O | | |
| succinylase | | | |
| Succinyl | NS26DP -> SUCC + | yodQ | |
| diaminopimelate | D26PIM | | |
| desuccinylase | | | |
| DephosphoCoA | DPCOA + ATP -> | ytaG | Same |
| kinase | ADP + COA | | |
| Isoprenyl | IPPP -> DMPP | ypgA | Same |
| pyrophosphate | | | |
| isomerase | | | |
| NAMN adenylyl | NAMN + ATP -> PPI + | yqeJ1 | |
| transferase | NAAD | | |
| Ketopantoate | AKP + NADPH -> | ylbQ | Slow |
| reductase | NADP + PANT | | |
| Phosphogluco- | G1P <-> G6P | ybbT | |
| mutase | | | |
| Ribose-5- | RL5P <-> R5P | ywlF | |
| phosphate | | | |
| isomerase A | | | |
| Transaldolase A | T3P1 + S7P <-> | ywjH | Same |
| | E4P + F6P | | |
| Hydroxymethyl- | 3HMGCOA -> ACCOA + | yngG | |
| glutaryl-CoA | AAC | | |
| lysase | | | |

Table 2 shows 11 reactions that were added to the B. subtilis metabolic reaction database and stoichiometric matrix based on putative assignments provided by the Subtilist genome database. The in silico B. subtilis model predicted that all of these reactions were essential for B. subtilis growth on glucose. Phenotypic studies using gene knockout studies on five of these genes have been performed by the European consortium group MICADO (MICrobial Advanced Database Organization; see, for example, Biaudet et al., Comput. Appl. Biosci. 13:431-438 (1997)) and include cardiolipin synthase, dephosphoCoA kinase, isoprenyl pyrophosphate isomerase, ketopantoate reductase and transaldolase A. Eleven reactions in Table 2 are also essential reactions. However, these reactions are slightly different from those in Table 1 in that at least some putative genes can be found. Deletion of any of the reactions in Table 2 should be lethal. However, the in vivo data (five reactions) which is shown in Column 4 of Table 2 indicated that they are not essential. Since the observed results from the gene deletion studies are inconsistent with the results predicted by the model, it is likely that the five genes are incorrectly assigned to the associated reactions.

The complete list of the 792 metabolic reactions included in the database, with the corresponding gene whose product catalyzes each reaction, is provided in Table 8. A list of abbreviations for the 525 metabolites that act as substrates and products of the reactions listed in Table 8 is provided in Table 9. The dimensions of the stoichiometric matrix including all reactions and reactants in the database is, therefore, 525×792. Individual exchange reactions (such as glucose and oxygen) and lumped demand exchange reaction (such as amylase and biomass) are not shown in the Tables 8 and 9 but are included in the reaction matrices for the specific simulations described below.

Thus, this example demonstrates that investigation of the metabolic biochemistry of B. subtilis using an in silico model of the invention can be useful for assigning pertinent biochemical reactions to sequences found in the genome; validating and scrutinizing annotation found in a genome database; and determining the presence of reactions or pathways in B. subtilis that are not indicated in the annotation of the B. subtilis genome or the biochemical literature.

### EXAMPLE II

This example shows how two parameters, the ratio of the number of ATP molecules produced per atom of oxygen (PO ratio), and the ATP maintenance requirement (M), can be determined using the B. subtilis metabolic model described in Example I.

The PO ratio and maintenance requirement (M) cannot be independently determined from fermentation studies alone because these two values are coupled as

m_{ATP}=m_{GLC}(12*PO+4) (Eq. 8)

where m_{ATP} is the mass of ATP, PO is the PO ratio and m_{GLC} is the mass of glucose consumed. The PO ratio is a molecular property which remains constant regardless of environmental conditions whereas the maintenance requirement is a macroscopic property which changes under different environmental conditions as described, for example, in Sauer and Bailey, Biotechnol. Bioeng. 64: 750-754 (1999). However, combinations of both parameters can be determined that are consistent with experimental data using the B. subtilis in silico model of the invention. The requirements for certain cellular building blocks, as listed in Table 3, were included in the metabolic flux analysis. The values in Table 3 were obtained from Dauner et al., Biotechnol. Bioeng. 76:132-143 (2001).

TABLE 3
REQUIREMENT (μmol/g DCW)

| Component | D = 0.11 hr⁻¹ | D = 0.44 hr⁻¹ |
|---|---|---|
| ATP | 35115 | 39440 |
| NADH | −3015 | −4052 |
| NADPH | 14405 | 14512 |
| CO2 | −2852 | −3011 |
| G6P | 712 | 444 |
| R5P | 445 | 644 |
| E4P | 397 | 460 |
| T3P (T3P1) | 428 | 235 |
| PGA (3PG) | 1241 | 1505 |
| PEP | 642 | 685 |
| PYR | 2994 | 3143 |
| ACA (ACCOA) | 2097 | 1524 |
| OAA (OA) | 1785 | 1998 |
| OGA (AKG) | 1236 | 1309 |
| SER | 262 | 304 |
| GLY | 542 | 629 |
| Cl | 411 | 549 |
| (not included) | | |
| Pi | 1640 | 1737 |
| NH4 (NH3) | 8066 | 9275 |
| SO4 (H2SO4) | 195 | 226 |

The values for certain extracellular fluxes, as listed in Table 4, were also included in the analysis. The values in Table 4 were obtained from Dauner et al., Biotechnol. Bioeng. 76:144-156 (2001).

TABLE 4
FLUX (mmol/g DCW/hr)

| Component | D = 0.11 hr⁻¹ | D = 0.44 hr⁻¹ |
|---|---|---|
| Riboflavin | 0.02 | 0.03 |
| Acetate | 0.01 | 0.09 |
| Citrate | 0.03 | 0.06 |
| Diaceytl | 0.09 | 0.17 |
| (Diacetoin) | | |
| Glucose | 1.98 | 7.05 |
| CO2 | 6.69 | 19.75 |
| O2 | 6.71 | 19.71 |

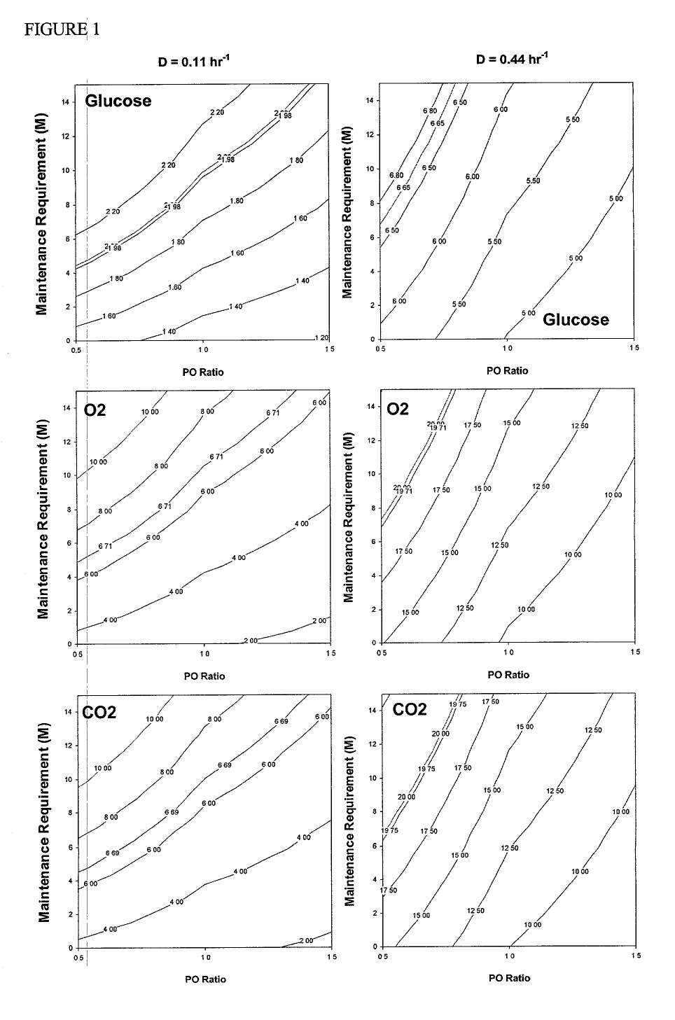

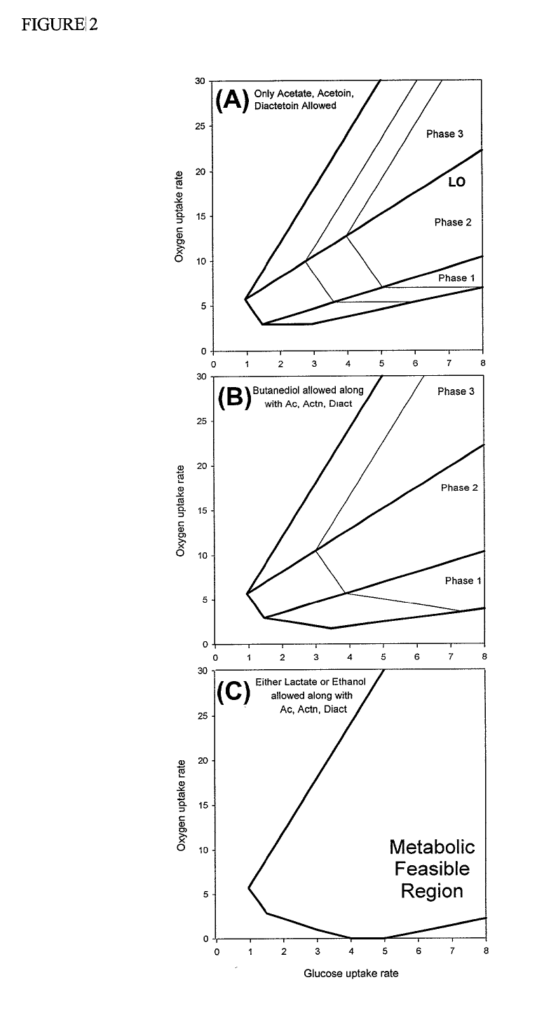

To estimate the values of P0 and M, linear programming (LP) was used to determine optimal flux for the in silico B. subtilis model. FIG. 1 shows the expected glucose uptake rate, O₂ uptake rate and CO₂ evolution rate as a function of PO and M at a growth rate (μ) of 0.11 hr⁻¹ or 0.44 hr⁻¹. The LP problem was repeatedly solved while varying the values of PO and M at a fixed value for μ. The objective function was to minimize the glucose uptake rate at given values of PO, M and μ. The values for certain extracellular fluxes, as listed in Table 4, were also included in the simulation as additional constraints. For example, at the dilution rate of 0.11 hr⁻¹, the riboflavin secretion rate was set at 0.11 mmol/g DCW/hr, the acetate secretion rate at 0.01 mmol/g DCW/hr, the citrate secretion rate at 0.03 mmol/g DCW/hr, and the diacetoin secretion rate at 0.09 mmol/g DCW/hr.

A combination of PO and M that minimize the following error function (sum of squares of weighted errors) was searched:

$$SSE = {( \frac{{q}_{GLC}^{m} - {q}_{GLC}^{e}}{{q}_{GLC}^{m}} )}^{2} + {( \frac{{q}_{O2}^{m} - {q}_{O2}^{e}}{{q}_{O2}^{m}} )}^{2} + {( \frac{{q}_{CO2}^{m} - {q}_{CO2}^{e}}{{q}_{CO2}^{m}} )}^{2} \\ {q}_{GLC}^{m} = measured glucose uptake rate \\ {q}_{O2}^{m} = measured oxygen uptake rate \\ {q}_{CO2}^{m} = measured cabon dioxide evolution rate \\ {q}_{GLC}^{e} = calculated glucose uptake rate \\ {q}_{O2}^{e} = calculated oxygen uptake rat \\ {q}_{CO2}^{e} = calculated cabon dioxide evolution rate \qquad ( Eq . 9 )$$

FIG. 1 shows contour diagrams for glucose uptake (top), oxygen uptake (middle), and carbon dioxide evolution (bottom) rates as a function of PO ratio and maintenance requirement. From the analysis, it was found that there were multiple solutions of the combinations of PO and M that fit with the experimental data. Using D=0.11 hr⁻¹, the best fit values were found at M=4.7 mmol ATP/g DCW/hr when PO=0.5, M=10.3 mmol ATP/g DCW/hr when PO=1.0, and M=16.9 mmol ATP/g DCW/hr when PO=1.5, as shown in FIG. 1B, which shows SSE as a function of M at different PO values. When D=0.44 hr⁻¹, the best fit values were found at M=6.6 mmol ATP/g DCW/hr when PO=0.5, M=23.3 mmol ATP/g DCW/hr when PO=1.0, and M=42.8 mmol ATP/g DCW/hr when PO=1.5. Therefore, no combination of PO and M that was consistent for both sets of experimental data was found. This discrepancy could be possibly due to experimental errors.

However, the genomic analysis of the electron transport system in B. subtilis suggests that the PO ratio is most likely close to 1. This is based on the assumptions that (1) only two electrons are transferred via the NADH dehydrogenase reactions without any proton translocation, (2) two protons are translocated per one electron by the cytochrome oxidase reactions, and (3) the ATP synthase reaction requires four protons to drive phosphorylation of one ATP molecule. This leads to the estimation of M to be 10.3 using the data of D=0.11 hr⁻¹. The estimated value of M=23.3 with the data of D=10.44 hr⁻¹ appears to be too high and, therefore, unlikely.

Thus, this example demonstrates use of an in silico B. subtilis model to predict the ATP maintenance requirement for optimal growth.

### EXAMPLE III

This example shows how the B. subtilis metabolic model can be used to calculate the range of characteristic phenotypes that the organism can display as a function of variations in the activity of multiple reactions.

For this analysis, O₂ and glucose uptake rates were defined as the two axes of the two-dimensional space. The optimal flux distribution was calculated using linear programming (LP) for all points in this plane by repeatedly solving the LP problem while adjusting the exchange fluxes defining the two-dimensional space. A finite number of quantitatively different metabolic pathway utilization patterns were identified in the plane, and lines were drawn to demarcate these regions. One demarcation line in the phenotypic phase plane (PhPP) was defined as the line of optimality (LO), and represents the optimal relation between the respective metabolic fluxes. The LO was identified by varying the x-axis (glucose uptake rate) and calculating the optimal y-axis (O₂ uptake rate), with the objective function defined as the growth flux. Further details regarding Phase-Plane Analysis are provided in Edwards et al., Biotechnol. Bioeng. 77: 27-36 (2002) and Edwards et al., Nature Biotech. 19:125-130 (2001)).

Lactate, acetoin, diacetoin, and butanediol were reported as fermentation byproducts from in vivo experimental results reported in the literature. Production of ethanol and succinate were not confirmed as fermentation byproducts in the reported in vivo experiments.

For each simulation, the maintenance requirement was constrained as M=9.5 mmol ATP/g DCW/hr, the PO ratio was 1 and ammonia was used as the nitrogen source.

FIG. 2A shows the results of the simulation where only acetoin, acetate, and diacetoin were allowed to be secreted as byproducts. In phase 1, both acetate and acetoin are secreted. In phase 2, only acetate is secreted. In phase 3, no organic acids are secreted and all carbon is converted to biomass or CO₂.

FIG. 2B shows the results of the simulation where butanediol along with acetoin, acetate, and diacetoin were allowed to be secreted as byproducts. In phase 1, acetate and butanediol are secreted. In phase 2, acetate is secreted. In phase 3, no organic acids are secreted. Note that no acetoin or diacetoin can be secreted under this condition.

FIG. 2C shows the results of the simulation where lactate (or ethanol) can be secreted along with acetoin, acetate, and diacetoin. The feasible metabolic region is slightly larger than in FIGS. 2A and 2B, and allows the O₂ uptake rate to be zero. However, B. subtilis is strictly aerobic unless nitrate or nitrite is provided. The phase plane in FIG. 2C shows that B. subtilis can be anaerobic only if the glucose uptake rate is in the range of 4 to 5 mmol/g DCW/hr. These results indicate that the reason why B. subtilis is a strict aerobic is due to its inability to secrete organic byproducts such as lactate ethanol and succinate that can supply the reducing equivalent, NADH. B. subtilis can metabolize TCA cycle intermediates as carbon substrates but no TCA cycle intermediates are found as byproducts. This means that the uptake systems for these metabolites work in only one direction and that the transporter systems involved in uptake of TCA cycle intermediates are different from those involved in secretion.

Thus, this example demonstrates that Phase Plane Analysis can be used to determine the optimal fermentation pattern for B. subtilis, and to determine the types of organic byproducts that can be accumulated under different oxygenation conditions and glucose uptake rates.

### EXAMPLE IV

This example shows how the B. subtilis metabolic model can be used to predict optimal flux distributions that would optimize fermentation performance, such as specific product yield or productivity. In particular, this example shows how flux based analysis (FBA) can be used to determine conditions that would maximize riboflavin, amylase (amyE), or protease (aprE) yields by B. subtilis grown on glucose.

The constraints on the system were set using the following assumptions set forth in Table 5.

TABLE 5

q_{glc} = 10 mmol/g DCW/hr (no limit on O₂)
PO ratio is set either at 0.5 or 1.0 or 1.5
M = 9.5 mmol ATP/g DCW/hr
One ATP needed to transport one molecule of protein
(amylase or subtilisin)
Biomass composition stays constant, and is the
composition of the growth rate at 0.11 hr⁻¹ (Table 1)
4.323 ATPs per peptide bond formed
Both Spase and SSPase are ATP independent, i.e. no ATP
needed to degrade the cleaved signal peptide into
individual amino acids

Table 6 shows the amino acid composition for amylase and subtilisin.

TABLE 6

| | |
|---|---|
| amylase (amyE) | subtilisin (aprE) |

| | Pre-process | Mature | Pre-process | Mature |
|---|---|---|---|---|
| | Form | Form | Form | Form |
| Ala | 57 | 50 | 47 | 44 |
| Arg | 25 | 24 | 5 | 4 |
| Asn | 56 | 56 | 18 | 17 |
| Asp | 44 | 44 | 13 | 13 |
| Cys | 1 | 1 | 0 | 0 |
| Gln | 29 | 29 | 14 | 13 |
| Glu | 25 | 25 | 12 | 12 |
| Gly | 53 | 51 | 37 | 37 |
| His | 17 | 16 | 8 | 8 |
| Ile | 35 | 35 | 21 | 19 |
| Leu | 44 | 36 | 22 | 17 |
| Lys | 33 | 31 | 25 | 23 |
| Met | 11 | 10 | 9 | 6 |
| Phe | 25 | 20 | 8 | 5 |
| Pro | 25 | 23 | 14 | 14 |
| Ser | 58 | 56 | 51 | 47 |
| Thr | 47 | 46 | 24 | 22 |
| Trp | 14 | 14 | 4 | 3 |
| Tyr | 28 | 28 | 16 | 16 |
| Val | 33 | 32 | 33 | 32 |
| ATP | 2853 | 2711 | 1647 | 1522 |

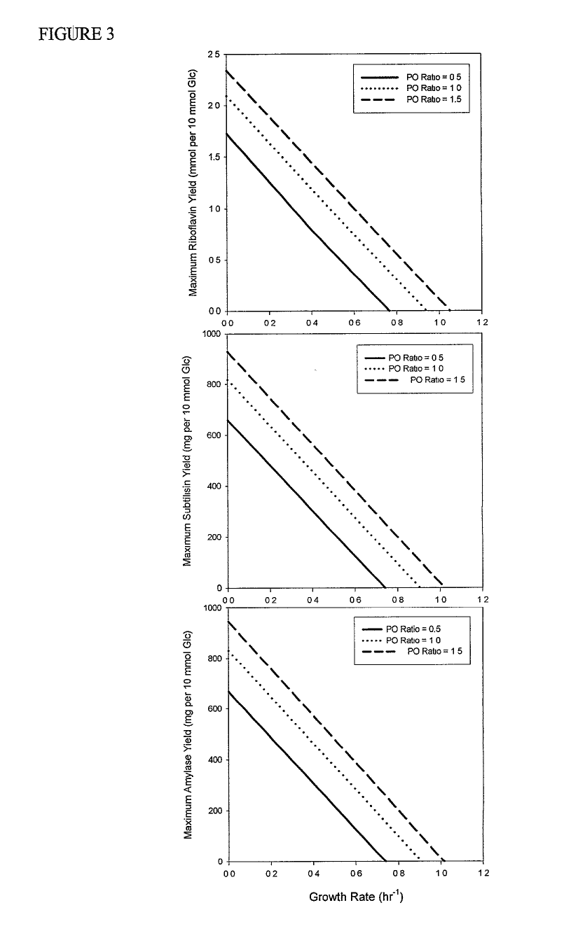

As shown in FIGS. 3A-C, yields of riboflavin, subtilisin and amylase, respectively, are lower at higher growth rates and at lower PO ratio. These results suggest that one metabolic engineering target is to increase the PO ratio to improve energetic efficiency of carbon substrate utilization.

FIG. 4A shows carbon flux distribution patterns at optimal yield for the above three different cases and optimal biomass case. The flux patterns are very different depending on the choice of objective functions, indicating that different metabolic optimization strategies are needed for different fermentation objectives.

The results shown in FIG. 4B suggest that in order to maximize riboflavin fermentation yield, high flux via the pentose phophate pathway (PPP) is required. The gene deletion study in Example V indicates that B. subtilis seems to possess very inefficient PPP. Therefore, the PPP will be a good metabolic engineering target to improve riboflavin fermentation yield.

Thus, this Example demonstrates use of an in silico B. subtilis model for the prediction of conditions for optimal production of riboflavin, amylase, or protease when B. subtilis is grown on glucose. This example further demonstrates use of the model to identify targets for engineering B. subtilis for improved fermentation yield.

### EXAMPLE V

This example shows how the B. subtilis metabolic model can be used to determine the effect of deletions of individual reactions in the network.

For this analysis, the objective function was the basic biomass function described in Table 3 in Example II except that the following additional metabolites were included in the biomass function: ALA, ARG, ASP, ASN, CYS, GLU, GLN, GLY, HIS, ILE, LEU, LYS, MET, PHE, PRO, THR, TRP, TYR, VAL, DATP, DGTP, DCTP, DTTP, GTP, CTP, UTP, PEPTIDO, PS, PE, CL, PG, THIAMIN, GLYTC1, GLYTC2, TEICHU, MTHF, SUCCOA, PTRC, Q, HEMEA, SHEME, FAD, NADP, SPMD. Since these metabolites were included in the modified biomass function at very low values, the quantitative changes in flux and growth rates due to this change in the biomass composition were insignificant. However, the addition of these biomass constituents ensured that the central and peripheral pathways leading to the synthesis of these metabolites were active and that inability to produce any of these metabolites would result in lethality. This representation is advantageous for determining the impact of deletion of a particular gene or reaction, that is not represented in the lumped biomass function, on the overall cell growth. For example, thiamin was not involved in calculating the composition of cellular building blocks in Example II. Therefore, in simulations with the lumped biomass function, the effect of deletions of thiamin biosynthetic genes cannot be addressed. However, thiamin serves as the coenzyme for a large number of enzyme systems in the metabolism of carbohydrates and amino acids such as pyruvate dehydrogenase, and deletions of any of the thiamin biosynthetic genes should be lethal.

For the simulations in this example, the uptake rates for oxygen, nitrogen, sulfate and phosphate were set very high and were essentially unlimited. The glucose uptake rate was set at 10 mmol/g DCW/hr. PO ratio was set at 1.375. The CYOA reaction was set not to generate protons as QH2+0.5 O2−>Q. Additionally, the constraints were set on the following reactions:

0<ATOB<10 (ATOB is irreversible)

PTA<ACxtI (acetate uptake rate)

ACS<ACxtI (acetate uptake rate)

KBL2<THRxtI (threonine uptake rate)

HUTH<HISxtI (histidine uptake rate)

MMCOAMT<LEUxtI (leucine uptake rate)

HMGCOAL<VALxtI (valine uptake rate)

SFCA (NAD-malic enzyme reaction)=0 (not active under glycolytic conditions)

MAEB (NADP-malic enzyme reaction)=0 (not active under glycolytic conditions)

PCKA (PEP carboxykinase reaction)=0 (not active under glycolytic conditions)

Simulations were conducted in which all 663 unique reactions were deleted one at a time. Of these, 252 reactions were determined to be essential for growth on glucose minimal medium. These results indicate that a high degree of redundancy exists in the B. subtilis metabolic network, such that inactivity of certain metabolic reactions can be compensated. The essential reactions are marked as “E” in Table 8.

It must be noted that a minimal reaction set is different from a minimal gene set for cellular growth and function. Also deletion of a reaction is different from deletion of a gene. For example, the ACEE reaction is a lumped reaction catalyzed by enzymes encoded by four genes, pdhABCD. Therefore, deletion of ACEE is equivalent to deletion of the four genes. Conversely, some genes encode enzymes that carry out multiple reactions. In these cases, deletion of any one of the associated reactions may not be lethal whereas deletion of the gene may be. For example, the adk (adenylate kinase) gene reaction is represented to catalyze four reactions: ADK1, ADK2, ADK3 and ADK4. Deletion of any of these reactions is not lethal for cell growth on glucose but deletion of the adk gene is lethal. At least one of the four ADK reactions is essential for growth on glucose minimal medium. In Table 8, all four ADK reactions are indicated as nonessential. Similar cases can be found in phosphate transport reactions (PIUP1 and PIUP2), CTP synthetase reactions (PYRG1 and PYRG2) and transketolase II reactions (TKTB1 and TKTB2) in which only one in each set is essential.

There are 17 reactions, marked “R” in Table 9, that were determined to be important for growth, in that their deletion led to growth retardation.

Table 7 shows a comparison of the results of the in silico gene deletion study with the experimental results of mutants grown on glucose minimal medium for some selected reactions in central carbon metabolism. As shown in Table 7, there exists a good qualitative correlation between the predicted in silico result and the observed experimental result.

TABLE 7

| | | | In silico | In vivo |
|---|---|---|---|---|
| Reaction | Enzyme | Stoichiomety | Prediction | Result |
| SUCA | 2-Ketoglutarate | AKG + NAD + | + | + |
| | dehyrogenase | COA -> CO2 + | + | + |
| | | NADH + SUCCOA | | |
| GND | 6- | D6PGC + NADP -> | + | No |
| | Phosphogluconate | NADPH + CO2 + | | data |
| | dehydrogenase | RL5P | | |
| | (decarboxylating) | | | |
| PGL | 6- | D6PL -> D6PGC | + | No |
| | Phosphoglucono- | | | data |
| | lactonase | | | |
| ACKA | Acetate kinase A | ACTP + ADP <-> | + | − |
| | | ATP + AC | | |
| ACS | Acetyl-CoA | ATP + AC + | + | No |
| | synthetase | COA -> AMP + | | data |
| | | PPI + ACCOA | | |
| ACNA | Aconitase A | CIT <-> ICIT | − | − |
| GLTA | Citrate synthase | ACCOA + OA -> | − | − |
| | | COA + CIT | | |
| ENO | Enolase | 2PG <-> PEP | Reduced | No |
| | | | | data |
| FBP | Fructose-1, 6- | FDP -> F6P + PI | + | |
| | bisphosphatase | | | |
| FBA | Fructose-1, 6- | FDP <-> T3P1 + | Reduced | No |
| | bisphosphatate | T3P2 | | data |
| FRDA | Fumurate | FUM + FADH -> | + | No |
| | reductase | SUCC + FAD | | data |
| GLK2 | Glucokinase | GLC + ATP -> | + | + |
| | | G6P + ADP | | |
| ZWF | Glucose 6- | G6P + NADP <-> | + | No |
| | phosphate-1- | D6PGL + NADPH | | data |
| | dehydrogenase | | | |
| GAPA | Glyceraldehyde- | T3PI + PI + | − | − |
| | 3-phosphate | NAD <-> | | |
| | dehydrogenase-A | NADH + 13DPG | | |
| | complex | | | |
| ICDA | Isocitrate | ICIT + NADP <-> | − | No |
| | dehydrogenase | CO2 + NADPH + | | data |
| | | AKG | | |
| MDH | Malate | MAL + NAD <-> | + | No |
| | dehydorgenase | NADH + OA | | data |
| PC | Pyruvate | PYR + CO2 -> | − | − |
| | carboxylase | OA + PI | | |
| PFKA | Phospofructo- | F6P + ATP -> | Reduced | No |
| | kinase | FDP + ADP | | data |
| PGI1 | Phosphoglucose | G6P <-> F6P | Reduced | Slow |
| | isomerase | | | |
| PGK | Phosphoglycerate | 13DPG + ADP <-> | − | − |
| | kinase | 3PG + ATP | | |
| PTA | Phosphotrans- | ACCOA + PI <-> | + | − |
| | acetylase | ACTP + COA | | |
| GPMA | Phosphoglycerate | 3PG <-> 2PG | Reduced | Very |
| | mutase | | | slow |
| ACEE | Pyruvate | PYR + COA + | − | − |
| | dehydrogenase | NAD -> NADH + | | |
| | | CO2 + ACCOA | | |
| PYKF | Pyruvate Kinase I | PEP + ADP -> | + | + |
| | | PYR + ATP | | |
| RPIA | Ribose-5- | RL5P <-> R5P | − | No |
| | phosphate | | | data |
| | isomerase A | | | |
| ARAD | Ribulose | RL5P <-> X5P | − | No |
| | phosphate 3- | | | data |
| | epimerase | | | |
| SDHA1 | Succinate | SUCC + FAD -> | + | No |
| | dehydrogenase | FADH + FUM | | data |
| TKTA1 | Transketolase I | R5P + X5P <-> | + | No |
| | | T3P1 + S7P | | data |
| TPIA | Triosphosphate | T3PI <-> T3P2 | Reduced | No |
| | Isomerase | | | data |

There exist some quantitative discrepancy in the phosphoglycerate mutase (GPM reaction) and phosphoglucose isomerase (PGI) reaction deletion cases. The in silico model predicts growth rates to be reduced by 14% and 2% in the phosphoglycerate mutase and phosphoglucose isomerase (PGI) cases, respectively. In both simulation cases, the growth rates are relatively unaffected despite blockage of the glycolytic steps because carbon metabolites can pass from the upper to the lower glycolytic metabolic pathways via the pentose phosphate pathway. When a GPM-deficient B. subtilis mutant was grown in glucose minimal media and observed in vivo, the mutant strain grew extremely slow at growth rates reduced by up to 90% (see, for example, Leyva-Vasquez and Setlow, J. Bacteriol. 176:3903-3910 (1994)). A PGI-deficient B. subtilis mutant grew at 42% of !the wild type growth rate (see, for example, Freese et al., Spores V. Halvorson et al. (ed.), American Society for Microbiology, Washington D.C. pp 212-224, (1972)). These results suggest that B. subtilis possess an inefficient pentose phosphate pathway which cannot compensate for the inactivity of glycolysis in these mutants.

There is a discrepancy in the acetate kinase A (ACKA) deletion case between the in silico simulation and in vivo results. In silico simulation predicted that the deletion of ACKA did not affect cell growth on glucose. When a ACKA mutant was grown in minimal medium with excess glucose, the growth rate was 33% of the wild type growth rate suggesting that acetate kinase A is important to deal with excess carbohydrate. It is likely that the blockage of acetate secretion leads to an accumulation of acetyl phosphate, which could be growth inhibitory. (See, for example, Grundy et al., J. Bacteriol. 175:7348-7355 (1993)). The effect of such inhibition and regulation was not accounted for in the current model.

There are about 1,800 genes in the B. subtilis genome for which no functional information is available. One of the approaches to assess gene function is a phenotypic analysis of mutants missing each one. Ogasawara constructed 789 such mutants for this purpose and observed phenotypic changes in 328 mutants under various conditions (Ogasawara, Res. Microbiol. 151: 129-134 (2000)). Ogasawara identified several novel essential genes that were not identified in previous genetic studies. Three of these genes were predicted to be essential by the in silico model including yybQ (inorganic pyrophosphatase), ispA or yqiD (farnesyl-diophosphate synthase) and dxs or yqiD (1-deoxyxylulose-5-phosphate synthase).

Thus, this example demonstrates that the in silico model can be used to uncover essential genes to augment or circumvent traditional genetic studies.

### EXAMPLE VI

This example demonstrates simulation of B. subtilis growth using a combined regulatory/metabolic model. This example further demonstrates the effects on growth rate prediction when regulation is represented in a B. subtilis metabolic model.

Glucose repression is a phenomenon of catabolite repression mediated by CcpA (catabolite control protein) in B. subtilis (see, for example, Grundy et al., J. Bacteriol. 175:7348-7355 (1993)). CcpA acts both as a negative regulator of carbohydrate (including, for example, arabinose and ribose) utilization genes and as a positive regulator of genes involved in excretion of excess carbon.

Growth of B. subtilis in the presence of glucose and arabinose was simulated as follows. The B. subtilis model described in Example I was modified to incorporate the logic statement:

Reg−ARAA=IF (Glucose exchange reaction) then NOT(ARAA).

The constraint for the ARAA reaction was:

(0)*Reg−ARAA≦R2≦(∞)*Reg−ARAA.

According to the logic statement if glucose is present the gene for L-arabinose isomerase is not expressed and the flux via reaction ARAA (L-arabinose isomerase, EC 5.3.1.4) is constrained to zero. When glucose is not present, for example, when it is consumed from the media, the flux via reaction ARAA has an infinite boundary value.

A simulation was run for the combined regulatory/metabolic model and the stand-alone metabolic model described in Example I. The following parameters were used: PO=1; M=9.5 mmol ATPs/g DCW/hr; glucose uptake rate=5 mmol/g DCW/hr; arabinose uptake rate=5 mmol/g DCW/hr; the flux via ribulose 5-phosphate isomerase reaction (converting ribulose 5-phosphate to xylose 5-phosphate) was constrained to be greater than zero; the uptake rates for oxygen, nitrogen, sulfate and phosphate were unconstrained; and the biomass composition was as set forth in Tables 3 and 4 for a growth rate at 0.11 hr⁻¹.

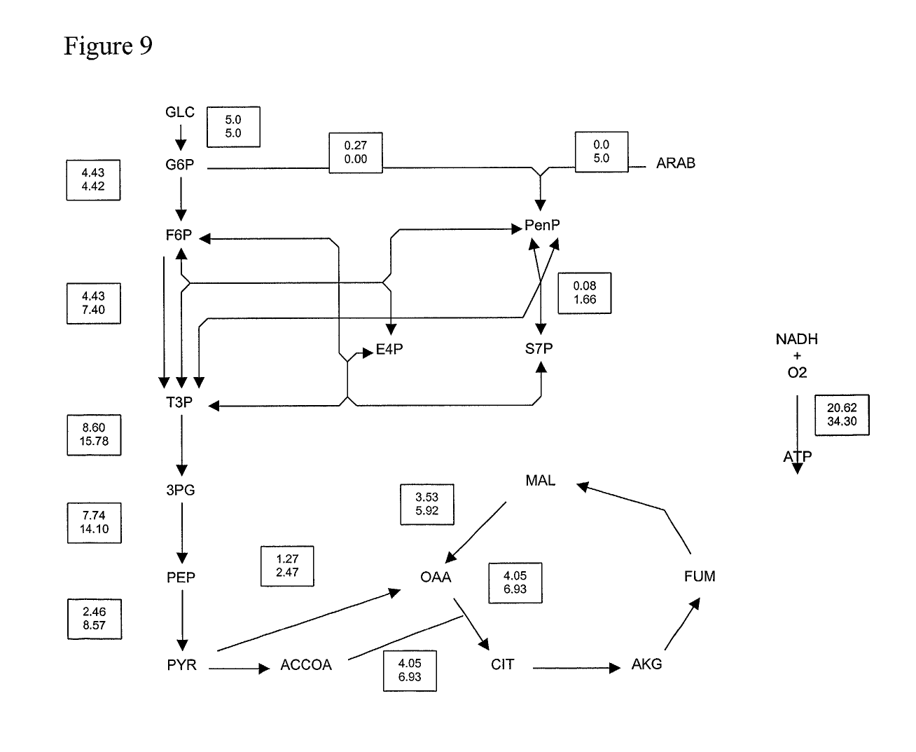

FIG. 9 shows the differences in network utilization between the regulated model (top numbers) and stand-alone model (bottom numbers). Absent consideration of repression mediated by CcpA, both glucose and arabinose were taken up and utilized in the simulation. However when regulation due to CcpA was included in the model, arabinose was not utilized due to the import and utilization of glucose. Comparison of the results for the non-regulated model with those for the regulated model indicated that regulation by CcpA resulted in lower cell growth rate. The predicted growth rate was 0.818 hr⁻¹ for the stand-alone metabolic model, and for the combined regulatory/metabolic model was 0.420 hr⁻¹.

Incorporation of the regulatory controls into the metabolic model can result in more accurate representations of the true physiology of the organism. Using the methods described in this example, molecular level regulatory knowledge as well as information about causal relationships, for example, where molecular detail is not known, can be incorporated into a B. subtilis model. As an example, in vivo studies of gene expression have identified 66 genes which are repressed by glucose but induced when glucose levels decrease (Yoshida et al., Nucl. Acids Res. 29:683-692 (2001)). Incorporation of regulation at each gene in response to glucose levels using boolean logic statements such as that demonstrated above for the ARAA reaction, can be used to increase the predictive capacity of a B. subtilis model.

Throughout this application various publications have been referenced. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention. Accordingly, the invention is limited only by the claims.

TABLE 8

| | | | | | Rxn |
|---|---|---|---|---|---|
| | | | | | Dele- |
| Gene Description | Gene | Reaction | Rxn Name | E. C. # | tion |
| EMP Pathway | | | | | |
| Enolase | eno | 2PG <-> PEP | ENO | 4.2.1.11 | R |
| Fructose-1,6-bisphosphatase | fbp | FDP -> F6P + PI | FBP | 3.1.3.11 | |
| Fructose-1,6-bisphosphatate aldolase | fbaA | FDP <-> T3P1 + T3P2 | FBA | 4.1.2.13 | R |
| Fructose-1,6-bisphosphatate aldolase | fbaB | FDP <-> T3P1 + T3P2 | FBA2 | 4.1.2.13 | |
| Glucokinase | glcK | GLC + ATP -> G6P + ADP | GLK2 | 2.7.1.2 | |
| Glucose-1-phosphate | glgC | ATP + G1P -> ADPGLC + PPI | GLGC | 2.7.7.27 | |
| adenylytransferase | | | | | |
| Glyceraldehyde-3-phosphate | gapA | T3P1 + PI + NAD <-> NADH + 13DPG | GAPA | 1.2.1.12 | E |
| dehydrogenase-A complex | | | | | |
| Glyceraldehyde-3-phosphate | gapB | T3P1 + PI + NAD <-> NADH + 13DPG | GAPC | 1.2.1.12 | |
| dehydrogenase-C complex | | | | | |
| Glycogen phosphorylase | glgP | GLYCOGEN + PI -> G1P | GLGP | 2.4.1.1 | |
| Glycogen synthase | glgA | ADPGLC -> ADP + GLYCOGEN | GLGA | 2.4.1.21 | |
| Methylglyoxal synthase | mgsA | T3P2 -> MTHGXL + PI | MGSA | 4.2.99.11 | |
| Phosphoenolpyruvate synthase | pps | PYR + ATP -> PEP + AMP + PI | PPSA | 2.7.9.2 | |
| Phosphofructokinase | pfkA | F6P + ATP -> FDP + ADP | PFKA | 2.7.1.11 | R |
| Phosphoglucose isomerase | pgi1 | G6P <-> F6P | PGI1 | 5.3.1.9 | R |
| Phosphoglucose isomerase | pgi2 | bDG6P <-> G6P | PGI2 | 5.3.1.9 | |
| Phosphoglucose isomerase | pgi3 | bDG6P <-> F6P | PGI3 | 5.3.1.9 | |
| Phosphoglycerate kinase | pgk | 13DPG + ADP <-> 3PG + ATP | PGK | 2.7.2.3 | E |
| Phosphoglycerate mutase 1 | pgm | 3PG <-> 2PG | GPMA | 5.4.2.1 | R |
| Phosphoglycerate mutase 2 | yhfR_{—} | 3PG <-> 2PG | GPMB | 5.4.2.1 | |
| Pyruvate dehydrogenase | pdhA | PYR + COA + NAD -> NADH + | ACEE | 1.2.4.1, | E |
| | | CO2 + ACCOA | | 2.3.1.12, | |
| | | | | 1.8.1.4 | |
| Pyruvate Kinase I | pyk | PEP + ADP -> PYR + ATP | PYKF | 2.7.1.40 | R |
| Triosphosphate Isomerase | tpiA | T3P1 <-> T3P2 | TPIA | 5.3.1.1 | R |
| Pentose Phosphate Pathway | | | | | |
| 2-Keto-3-deoxy-6-phosphogluconate | kdgA | 2KD6PG -> T3P1 + PYR | EDA | 4.1.2.14 | |
| aldolase | | | | | |
| 6-Phosphogluconate dehydrogenase | gntZ | D6PGC + NADP -> NADPH + | GND | 1.1.1.44 | |
| (decarboxylating) | | CO2 + RL5P | | | |
| 6-Phosphogluconate dehydrogenase | yqjI | D6PGC + NADP -> NADPH + | GND2 | 1.1.1.44 | |
| (decarboxylating) | | CO2 + RL5P | | | |
| 6-Phosphogluconolactonase | PGL | D6PGL -> D6PGC | PGL | 3.1.1.31 | |
| Glucose 6-phosphate-1-dehydrogenase | zwf | G6P + NADP <-> D6PGL + NADPH | ZWF | 1.1.1.49 | |
| Ribose-5-phosphate isomerase A | ywlF | RL5P <-> R5P | RPIA | 5.3.1.6 | E |
| Ribulose phosphate 3-epimerase | rpe | RL5P <-> X5P | RPE | 5.1.3.1 | |
| Transaldolase A | ywjH | T3P1 + S7P <-> E4P + F6P | TALA | 2.2.1.2 | |
| Transketolase II | tkt1 | R5P + X5P <-> T3P1 + S7P | TKTB1 | 2.2.1.1 | |
| Transketolase II | tkt2 | X5P + E4P <-> F6P + T3P1 | TKTB2 | 2.2.1.1 | |
| The Tricarboxylic Acid Cycle | | | | | |
| 2-Ketoglutarate dehyrogenase | odhA | AKG + NAD + COA -> CO2 + | SUCA | 1.2.4.2, 2.3.1. | R |
| | | NADH + SUCCOA | | 61, 1.8.1.4 | |
| Aconitase A | citB | CIT <-> ICIT | ACNA | 4.2.1.3 | E |
| Citrate synthase | citA | ACCOA + OA -> COA + CIT | GLTA | 4.1.3.7 | E |
| Citrate synthase | citZ | ACCOA + OA -> COA + CIT | GLTA2 | 4.1.3.7 | |
| Citrate synthase | mmgD | ACCOA + OA -> COA + CIT | GLTA3 | 4.1.3.7 | |
| Fumarase A | citG | FUM <-> MAL | FUMA | 4.2.1.2 | E |
| Fumurate reductase | sdhA3 | FUM + FADH -> SUCC + FAD | FRDA | 1.3.99.1 | |
| Isocitrate dehydrogenase | icd | ICIT + NADP <-> CO2 + | ICDA | 1.1.1.42 | E |
| | | NADPH + AKG | | | |
| Malate dehydrogenase | malS | MAL + NAD <-> NADH + OA | MDH | 1.1.1.37 | E |
| Malate dehydrogenase | mdh | MAL + NAD <-> NADH + OA | MDH2 | 1.1.1.37 | |
| Succinate dehydrogenase (Combine w | sdhA1 | SUCC + FAD -> FADH + FUM | SDHA1 | 1.3.99.1 | |
| FRDA) | | | | | |
| Succinyl-CoA synthetase | sucD | SUCCOA + ADP + PI <-> ATP + | SUCC | 6.2.1.5 | R |
| | | COA + SUCC | | | |
| Pyruvate Metabolism | | | | | |
| Acetaldehyde dehydrogenase | aldX4 | ACCOA + 2 NADH <-> ETH + | ADHE2 | 1.2.1.10 | |
| | | 2 NAD + COA | | | |
| Acetaldehyde dehydrogenase | aldY4 | ACCOA + 2 NADH <-> ETH + | ADHE3 | | |
| | | 2 NAD + COA | | | |
| Acetate kinase A | ackA | ACTP + ADP <-> ATP + AC | ACKA | 2.7.2.1 | |
| Acetyl-CoA synthetase | acsA | ATP + AC + COA -> AMP + | ACS | 6.2.1.1 | |
| | | PPI + ACCOA | | | |
| Acetyl-CoA synthetase | ytcl | ATP + AC + COA -> AMP + | ACS2 | 6.2.1.1 | |
| | | PPI + ACCOA | | | |
| Formate hydrogen lyase | fdhD | FOR -> CO2 | FDHF1 | 1.2.1.2, | E |
| | | | | 1.12.1.2 | |
| L-Lactate dehydrogenase | ldh | PYR + NADH <-> NAD + LLAC | LDH | 1.1.1.27 | |
| Phosphotransacetylase | pta | ACCOA + PI <-> ACTP + COA | PTA | 2.3.1.8 | |
| Anaplerotic Reactions | | | | | |
| Inorganic pyrophosphatase | ppaC | PPI -> 2 PI | PPA | 3.6.1.1 | E |
| Malic enzyme (NAD) | mleA1 | MAL + NAD -> CO2 + NADH + PYR | SFCA | 1.1.1.38 | |
| Malic enzyme (NADP) | mleA2 | MAL + NADP -> CO2 + NADPH + PYR | MAEB | 1.1.1.40 | |
| Phosphoenolpyruvate carboxykinase | pckA | OA + ATP -> PEP + CO2 + ADP | PCKA | 4.1.1.49 | |
| Pyruvate carboxylase | pycA | PYR + ATP + CO2 -> OA + PI + ADP | PC | 6.4.1.1 | E |
| Respiration (Note: the P/O ratio is set to 1.5 as an | | | | | |
| example) | | | | | |
| Cytochrome oxidase bd | ctaD | QH2 + .5 O2 -> Q + 2 HEXT | CYDA | 1.10.2.2, | R |
| | | | | 1.9.3.1 | |
| Cytochrome oxidase bo3 | ctaC | QH2 + .5 O2 -> Q + 2.5 HEXT | CYOA | 1.10.2.2, | |
| | | | | 1.9.3.1 | |
| F0F1-ATPase | atpA | ATP <-> ADP + PI + 4 HEXT | ATPA | 3.6.1.34 | R |
| Glycerol-3-phosphate dehydrogenase | glpD | GL3P + Q -> T3P2 + QH2 | GLPD | 1.1.99.5 | |
| (aerobic) | | | | | |
| NADH dehydrogenase I | ndhF1 | NADH + Q -> NAD + QH2 + 3.5 HEXT | NUOA | 1.6.5.3 | R |
| NADH dehydrogenase II | ndhF2 | NADH + Q -> NAD + QH2 | NDH | 1.6.5.3 | |
| Succinate dehydrogenase complex | sdhA2 | FADH + Q <-> FAD + QH2 | SDHA2 | 1.3.5.1 | R |
| Thioredoxin reductase | trxB | OTHIO + NADPH -> NADP + RTHIO | TRXB | 1.6.4.5 | E |
| Alternative Carbon Source | | | | | |
| Melibiose | | | | | |
| Alpha-galactosidase (melibiase) | melA | MELI -> GLC + GLAC | MELA | 3.2.1.22 | |
| Galactose | | | | | |
| Galactokinase | galK | GLAC + ATP -> GAL1P + ADP | GALK | 2.7.1.6 | |
| Galactose-1-phosphate | galT | GAL1P + UDPG <-> G1P + UDPGAL | GALT | 2.7.7.10 | |
| uridylyltransferase | | | | | |
| UDP-glucose 4-epimerase | galE | UDPGAL <-> UDPG | GALE | 5.1.3.2 | |
| UDP-glucose 4-epimerase | yjaV | UDPGAL <-> UDPG | GALE2 | 5.1.3.2 | |
| UDP-glucose-1-phosphate | gtaB | G1P + UTP <-> UDPG + PPI | GALU1 | 2.7.7.9 | E |
| uridylyltransferase | | | | | |
| UDP-glucose-1-phosphate | ytdA | G1P + UTP <-> UDPG + PPI | GALU1a | 2.7.7.9 | |
| uridylyltransferase | | | | | |
| UDP-glucose-1-phosphate | yngB | G1P + UTP <-> UDPG + PPI | GALU1b | 2.7.7.9 | |
| uridylyltransferase | | | | | |
| Phosphoglucomutase | ybbT | G1P <-> G6P | PGM | 5.4.2.2 | E |
| Lactose | | | | | |
| Periplasmic beta-glucosidase precursor | bglH | LCTS -> GLC + GLAC | BGLX | 3.2.1.21 | |
| phospho-beta-glucosidase | yesZ | LCTS -> GLC + GLAC | BGLX2 | 3.2.1.21 | |
| phospho-beta-glucosidase | ydhP | LCTS -> GLC + GLAC | BGLX3 | 3.2.1.21 | |
| Beta-galactosidase (LACTase) | yckE | LCTS -> GLC + GLAC | LACZ | 3.2.1.23 | |
| Beta-galactosidase (LACTase) | lacA | LCTS -> GLC + GLAC | LACZ2 | 3.2.1.23 | |
| Trehalose | | | | | |
| trehalose-6-phosphate hydrolase | treA | TRE6P -> bDG6P + GLC | TREC | 3.2.1.93 | |
| Fructose | | | | | |
| 1-Phosphofructokinase (Fructose 1- | fruK | F1P + ATP -> FDP + ADP | FRUK1 | 2.7.1.56 | |
| phosphate kinase) | | | | | |
| Xylose isomerase | xylA2 | FRU -> GLC | XYLA1 | 5.3.1.5 | |
| Mannose | | | | | |
| Phosphomannomutase | yhxB | MAN6P <-> MAN1P | CPSG | 5.4.2.8 | |
| Mannose-6-phosphate isomerase | manA | MAN6P <-> F6P | MANA | 5.3.1.8 | |
| Mannose-6-phosphate isomerase | pmi | MAN6P <-> F6P | MANA2 | 5.3.1.8 | |
| Mannose-6-phosphate isomerase | ydhS | MAN6P <-> F6P | MANA3 | 5.3.1.8 | |
| N-Acetylglucosamine | | | | | |
| N-Acetylglucosamine-6-phosphate | nagA | NAGP -> GA6P + AC | NAGA | 3.5.1.25 | |
| deacetylase | | | | | |
| Glucosamine | | | | | |
| Glucosamine-6-phosphate deaminase | gamA | GA6P -> F6P + NH3 | NAGB | 5.3.1.10 | |
| Fucose | | | | | |
| Aldehyde dehydrogenase A | aldX3 | LACAL + NAD <-> LLAC + NADH | ALDA | 1.2.1.22 | |
| Aldehyde dehydrogenase B | aldY3 | LACAL + NAD <-> LLAC + NADH | ALDB | 1.2.1.22 | |
| Aldehyde dehydrogenase | ycbD | LACAL + NAD <-> LLAC + NADH | ADHC1 | 1.1.1.1 | E |
| Aldehyde dehydrogenase | aldX2 | GLAL + NADH <-> GL + NAD | ADHC2 | 1.1.1.1 | E |
| Aldehyde dehydrogenase | aldY2 | GLAL + NADH <-> GL + NAD | ADHC3 | 1.1.1.1 | |
| Aldehyde dehydrogenase | aldX1 | ACAL + NAD -> AC + NADH | ALDH2 | 1.2.1.3 | |
| Aldehyde dehydrogenase | aldY1 | ACAL + NAD -> AC + NADH | ADHC4 | 1.2.1.3 | E |
| Aldehyde dehydrogenase | dhaS | ACAL + NAD -> AC + NADH | ADHC5 | 1.2.1.3 | |
| Aldehyde dehydrogenase | ywdH | ACAL + NAD -> AC + NADH | ADHC6 | 1.2.1.3 | |
| Gluconate | | | | | |
| Gluconokinase I | gntK | GLCN + ATP -> D6PGC + ADP | GNTV | 2.7.1.12 | |
| Rhamnose | | | | | |
| L-Rhamnose isomerase | yulE | RMN <-> RML | RHAA | 5.3.1.14 | |
| Rhamnulokinase | yulC | RML + ATP -> RML1P + ADP | RHAB | 2.7.1.5 | |
| Arabinose | | | | | |
| L-Arabinose isomerase | araA | ARAB <-> RBL | ARAA | 5.3.1.4 | |
| L-Ribulokinase | araB1 | RBL + ATP -> LRL5P + ADP | ARAB | 2.7.1.16 | |
| L-Ribulokinase | araB2 | RBL + ATP -> RL5P + ADP | ARAB | 2.7.1.16 | |
| L-Ribulose-phosphate 4-epimerase | araD | LRL5P <-> X5P | ARAD | 5.1.3.4 | |
| Xylose | | | | | |
| Xylose isomerase | xylA | XYL <-> XUL | XYLA2 | 5.3.1.5 | |
| Xylulokinase | xylB | XUL + ATP -> X5P + ADP | XYLB | 2.7.1.17 | |
| Xylulokinase | yoaC | XUL + ATP -> X5P + ADP | XYLB2 | | |
| Ribose | | | | | |
| Ribokinase | rbsK | RIB + ATP -> R5P + ADP | RBSK | 2.7.1.15 | |
| Ribokinase | yurL | RIB + ATP -> R5P + ADP | RBSK2 | 2.7.1.15 | |
| Mannitol | | | | | |
| Mannitol-1-phosphates 5- | mtlD | MNT6P + NAD <-> F6P + NADH | MTLD | 1.1.1.17 | |
| dehydrogenase | | | | | |
| Glycerol | | | | | |
| Glycerol kinase | glpK | GL + ATP -> GL3P + ADP | GLPK | 2.7.1.30 | E |
| Glycerol-3-phosphate-dehydrogenase- | gpsA | GL3P + NADP <-> T3P2 + NADPH | GPSA1 | 1.1.1.94 | E |
| [NAD(P)+] | | | | | |
| Nucleosides and Deoxynucleosides | | | | | |
| Phosphopentomutase | drm1 | DR1P <-> DR5P | DEOB1 | 5.4.2.7 | |
| Phosphopentomutase | drm2 | R1P <-> R5P | DEOB2 | 5.4.2.7 | |
| Deoxyribose-phosphate aldolase | dra | DR5P -> ACAL + T3P1 | DEOC | 4.1.2.4 | |
| Aspartate & Asparagine Biosynthesis | | | | | |
| Asparagine synthetase (Glutamate | asnB | ASP + ATP + GLN -> GLU + ASN + | ASNB1 | 6.3.5.4 | E |
| dependent) | | AMP + PPI | | | |
| Asparagine synthetase (Glutamate | asnH | ASP + ATP + GLN -> GLU + ASN + | ASNB1b | 6.3.5.4 | |
| dependent) | | AMP + PPI | | | |
| Asparagine synthetase (Glutamate | asnO | ASP + ATP + GLN -> GLU + ASN + | ASNB1c | 6.3.5.4 | |
| dependent) | | AMP + PPI | | | |
| Asparate transaminase | aspB | OA + GLU <-> ASP + AKG | ASPC1 | 2.6.1.1 | E |
| Asparate transaminase | yhdR_{—} | OA + GLU <-> ASP + AKG | ASPC2 | 2.6.1.1 | |
| Asparate transaminase | ykrV | OA + GLU <-> ASP + AKG | ASPC3 | 2.6.1.1 | |
| Asparate transaminase | yurG | OA + GLU <-> ASP + AKG | ASPC4 | 2.6.1.1 | |
| Glutamate and Glutamine Biosynthesis | | | | | |
| Glutamate dehydrogenase | gudB | AKG + NH3 + NADPH <-> | GDHA | 1.4.1.4 | R |
| | | GLU + NADP | | | |
| Glutamate dehydrogenase II | rocG | AKG + NH3 + NADH <-> GLU + NAD | GDHA2 | | R |
| Glutamate synthase | yerD | AKG + GLN + NADPH -> | GLTB | 1.4.1.13 | |
| | | NADP + 2 GLU | | | |
| Glutamate synthase: NADH specific | gltA | AKG + GLN + NADH -> NAD + 2 GLU | GLTB2 | 1.4.1.13 | |
| Glutamate-ammonia ligase | glnA | GLU + NH3 + ATP -> GLN + ADP + PI | GLNA | 6.3.1.2 | E |
| Alanine Biosynthesis | | | | | |
| Alanine racemase, biosynthetic | alr_{—} | ALA <-> DALA | ALR_{—} | 5.1.1.1 | |
| Alanine racemase, catabolic | yncD | ALA -> DALA | DADX | 5.1.1.1 | |
| Alanine transaminase | alaT | PYR + GLU <-> AKG + ALA | ALAB | 2.6.1.2 | E |
| Arginine, Putriscine, and Spermidine Biosynthesis | | | | | |
| 5-Methylthioribose kinase | MTHRKN | 5MTR + ATP -> 5MTRP + ADP | MTHRKN | 2.7.1.100 | E |
| 5-Methylthioribose-1-phosphate | MTHIPIS | 5MTRP <-> 5MTR1P | MTHIPIS | 5.3.1.23 | E |
| isomerase | | | | | |
| Acetylornithine deacetylase | ylmB1 | NAARON -> AC + ORN | ARGE1 | 3.5.1.16 | |
| Acetylornithine transaminase | argD | NAGLUSAL + GLU <-> | ARGD | 2.6.1.11 | E |
| | | AKG + NAARON | | | |
| Adenosylmethionine decarboxylase | speD | SAM <-> DSAM + CO2 | SPED | 4.1.1.50 | E |
| Agmatinase | speB | AGM -> UREA + PTRC | SPEB | 3.5.3.11 | E |
| Arginine decarboxylase, biosynthetic | speA | ARG -> CO2 + AGM | SPEA | 4.1.1.19 | E |
| Argininosuccinate lyase | argH | ARGSUCC <-> FUM + ARG | ARGH | 4.3.2.1 | E |
| Argininosuccinate synthase | argG | CITR + ASP + ATP -> AMP + | ARGG | 6.3.4.5 | E |
| | | PPI + ARGSUCC | | | |
| Carbamoyl phosphate synthetase | carA | GLN + 2 ATP + CO2 -> GLU + CAP + | CARA | 6.3.5.5 | E |
| | | 2 ADP + PI | | | |
| E-1 (Enolase-phosphatase) | NE1PH | 5MTR1P -> DKMPP | NE1PH | | E |
| E-3 (Unknown) | NE3UNK | DKMPP -> FOR + KMB | NE3UNK | | E |
| Methylthioadenosine nucleosidase | mtn | 5MTA -> AD + 5MTR | MTHAKN | 3.2.2.16 | E |
| N-Acetylglutamate kinase | argB | NAGLU + ATP -> ADP + NAGLUYP | ARGB | 2.7.2.8 | E |
| N-Acetylglutamate phosphate | argC | NAGLUYP + NADPH <-> | ARGC | 1.2.1.38 | E |
| reductase | | NADP + PI + NAGLUSAL | | | |
| N-Acetylglutamate synthase | ARGA | GLU + ACCOA -> COA + NAGLU | ARGA | 2.3.1.1 | |
| Ornithine carbamoyl transferase 1 | argF | ORN + CAP <-> CITR + PI | ARGF | 2.1.3.3 | E |
| Ornithine transaminase | rocD | ORN + AKG -> GLUGSAL + GLU | YGJG | 2.6.1.13 | |
| Spermidine synthase | speE | PTRC + DSAM -> SPMD + 5MTA | SPEE | 2.5.1.16 | E |
| Transamination (Unknown) | TNSUNK | KMB + GLN -> GLU + MET | TNSUNK | | E |
| Urease | ureA | UREA -> CO2 + 2 NH3 | UREA | 3.5.1.5 | E |
| Proline biosynthesis | | | | | |
| γ-Glutamyl kinase | proB | GLU + ATP -> ADP + GLUP | PROB | 2.7.2.11 | |
| γ-Glutamyl kinase | proJ | GLU + ATP -> ADP + GLUP | PROB2 | 2.7.2.11 | |
| Glutamate-5-semialdehyde | proA | GLUP + NADPH -> NADP + | PROA | 1.2.1.41 | |
| dehydrogenase | | PI + GLUGSAL | | | |
| Pyrroline-5-carboxylate reductase | proG | GLUGSAL + NADPH -> PRO + NADP | PROC | 1.5.1.2 | E |
| Pyrroline-5-carboxylate reductase | proH | GLUGSAL + NADPH -> PRO + NADP | PROC2 | 1.5.1.2 | |
| Pyrroline-5-carboxylate reductase | proI | GLUGSAL + NADPH -> PRO + NADP | PROC3 | 1.5.1.2 | |
| Branched Chain Amino Acid | | | | | |
| Biosynthesis | | | | | |
| 3-Isopropylmalate dehydrogenase | leuB | IPPMAL + NAD -> NADH + | LEUB | 1.1.1.85 | E |
| | | OICAP + CO2 | | | |
| 3-Isopropylmalate dehydrogenase | ycsA | IPPMAL + NAD -> NADH + | LEUB2 | 1.1.1.85 | |
| | | OICAP + CO2 | | | |
| Acetohydroxy Acid isomeroreductase | ilvC1 | ABUT + NADPH -> NADP + DHMVA | ILVC1 | 1.1.1.86 | E |
| Acetohydroxy acid isomeroreductase | ilvC2 | ACLAC + NADPH -> NADP + DHVAL | ILVC2 | 1.1.1.86 | E |
| Acetohydroxybutanoate synthase I | alsS2 | OBUT + PYR -> ABUT + CO2 | ILVB1 | 4.1.3.18 | E |
| Acetohydroxybutanoate synthase II | ilvB2 | OBUT + PYR -> ABUT + CO2 | ILVG1 | 4.1.3.18 | |
| Acetolactate synthase I | alsS1 | 2 PYR -> CO2 + ACLAC | ILVB2 | 4.1.3.18 | E |
| Acetolactate synthase II | ilvB1 | 2 PYR -> CO2 + ACLAC | ILVG2 | 4.1.3.18 | |
| Branched chain amino acid | ywaA1 | OMVAL + GLU <-> AKG + ILE | ILVE1 | 4.6.1.42 | E |
| aminotransferase | | | | | |
| Branched chain amino acid | ybgE1 | OMVAL + GLU <-> AKG + ILE | ILVE1b | | |
| aminotransferase | | | | | |
| Branched chain amino acid | ywaA2 | OIVAL + GLU <-> AKG + VAL | ILVE2 | 2.6.1.42 | E |
| aminotransferase | | | | | |
| Branched chain amino acid | ybgE2 | OIVAL + GLU <-> AKG + VAL | ILVE2b | | |
| aminotransferase | | | | | |
| Branched chain amino acid | ywaA3 | OICAP + GLU <-> AKG + LEU | ILVE4 | 2.6.1.42 | E |
| aminotransferase | | | | | |
| Branched chain amino acid | ybgE3 | OICAP + GLU <-> AKG + LEU | ILVE4b | | |
| aminotransferase | | | | | |
| Dihydroxy acid dehydratase | ilvD1 | DHMVA -> OMVAL | ILVD1 | 4.2.1.9 | E |
| Dihydroxy acid dehydratase | ilvD2 | DHVAL -> OIVAL | ILVD2 | 4.2.1.9 | E |
| Isopropylmalate isomerase | leuC | CBHCAP <-> IPPMAL | LEUC | 4.2.1.33 | E |
| Isopropylmalate synthase | leuA | ACCOA + OIVAL -> COA + CBHCAP | LEUA | 4.1.3.12 | E |
| Threonine dehydratase, biosynthetic | ilvA | THR -> NH3 + OBUT | ILVA | 4.2.1.16 | E |
| Aromatic Amino Acids | | | | | |
| 2-Dehydro-3-deoxyphosphoheptonate | aroA1 | E4P + PEP -> PI + 3DDAH7P | AROF | 4.1.2.15 | E |
| aldolase F | | | | | |
| 3-Dehydroquinate dehydratase | aroC | DQT <-> DHSK | AROD | 4.2.1.10 | E |
| 3-Dehydroquinate dehydratase | yqhS | DQT <-> DHSK | AROD2 | 4.2.1.10 | |
| 3-Dehydroquinate synthase | aroB | 3DDAH7P -> DQT + PI | AROB | 4.6.1.3 | E |
| 3-Phosphoshikimate-1- | aroE | SME5P + PEP <-> 3PSME + PI | AROA | 2.5.1.19 | E |
| carboxyvinyltransferase | | | | | |
| Anthranilate synthase | trpE | CHOR + GLN -> GLU + PYR + AN | TRPDE | 4.1.3.27 | |
| Anthranilate synthase component II | trpD | AN + PRPP -> PPI + NPRAN | TRPD | 2.4.2.18 | E |
| Aromatic amino acid transaminase | hisC2 | PHPYR + GLU <-> AKG + PHE | TYRB1 | 2.6.1.57 | E |
| Aromatic amino acid transaminase | hisC3 | HPHPYR + GLU <-> AKG + TYR | TYRB2 | 2.6.1.5 | E |
| Chorismate mutase 1 | aroA2 | CHOR -> PHEN | PHEA1 | 5.4.99.5 | E |
| Chorismate mutase 2 | aroH | CHOR -> PHEN | TYRA1 | 5.4.99.5 | |
| Chorismate mutase 2 | pheB | CHOR -> PHEN | TYRA1b | 5.4.99.5 | |
| Chorismate synthase | aroF | 3PSME -> PI + CHOR | AROC | 4.6.1.4 | E |
| Indoleglycerol phosphate synthase | trpC | CPAD5P -> CO2 + IGP | TRPC2 | 4.1.1.48 | E |
| Phosphoribosyl anthranilate isomerase | trpF | NPRAN -> CPAD5P | TRPC1 | 5.3.1.24 | E |
| Phosphoribosyl anthranilate isomerase | ynal | NPRAN -> CPAD5P | TRPC1b | 5.3.1.24 | |
| Prephanate dehydrogenase | tyrA | PHEN + NAD -> HPHPYR + | TYRA2 | 1.3.1.12 | E |
| | | CO2 + NADH | | | |
| Prephenate dehydratase | pheA | PHEN -> CO2 + PHPYR | PHEA2 | 4.2.1.51 | E |
| Shikimate dehydrogenase | aroD | DHSK + NADPH <-> SME + NADP | AROE | 1.1.1.25 | E |
| Shikimate kinase I | aroK | SME + ATP -> ADP + SME5P | AROK | 2.7.1.71 | E |
| Tryptophan synthase | trpA | IGP + SER -> T3P1 + TRP | TRPA | 4.2.1.20 | E |
| Histidine Biosynthesis | | | | | |
| ATP phosphoribosyltransferase | hisG | PRPP + ATP -> PPI + PRBATP | HISG | 2.4.2.17 | E |
| Histidinol dehydrogenase | hisD | HISOL + 3 NAD -> HIS + 3 NADH | HISD | 1.1.1.23 | E |
| Histidinol phosphatase | hisJ | HISOLP -> PI + HISOL | HISB2 | 3.1.3.15 | E |
| Imidazoleglycerol phosphate | hisB | DIMGP -> IMACP | HISB1 | 4.2.1.19 | E |
| dehydratase | | | | | |
| Imidazoleglycerol phosphate synthase | hisF | PRLP + GLN -> GLU + | HISF | 2.4.2.— | E |
| | | AICAR + DIMGP | | | |
| L-Histidinol phosphate | hisC1 | IMACP + GLU -> AKG + HISOLP | HISC | 2.6.1.9 | E |
| aminotransferase | | | | | |
| Phosphoribosyl pyrophosphate | prs | R5P + ATP <-> | PRSA | 2.7.6.1 | E |
| synthase | | PRPP + AMP | | | |
| Phosphoribosyl-AMP cyclohydrolase | hisI1 | PRBAMP -> PRFP | HISI2 | 3.5.4.19 | E |
| Phosphoribosyl-ATP pyrophosphatase | hisI2 | PRBATP -> PPI + PRBAMP | HISI1 | 3.6.1.31 | E |
| Phosphoribosylformimino-5-amino-1- | hisA | PRFP -> PRLP | HISA | 5.3.1.16 | E |
| phosphoribosyl-4-imidazole | | | | | |
| carboxamide isomerase | | | | | |
| Serine & Glycine Biosynthesis | | | | | |
| 3-Phosphoglycerate dehydrogenase | serA | 3PG + NAD -> NADH + PHP | SERA | 1.1.1.95 | E |
| 3-Phosphoglycerate dehydrogenase | yoaD | 3PG + NAD -> NADH + PHP | SERA2 | 1.1.1.95 | |
| Glycine hydroxymethyltransferase | glyA3 | THF + SER -> GLY + METTHF | GLYA3 | 2.1.2.1 | |
| Phosphoserine phosphatase | SERB | 3PSER -> PI + SER | SERB | 3.1.3.3 | E |
| Phosphoserine transaminase | serC | PHP + GLU -> AKG + 3PSER | SERC1 | 2.6.1.52 | E |
| Cysteine Biosynthesis | | | | | |
| 3′-5′ Bisphosphate nucleotidase | BISPHDS | PAP -> AMP + PI | BISPHDS | 3.1.3.7 | E |
| 3′-Phospho-adenylylsulfate reductase | cysH | PAPS + RTHIO -> OTHIO + | CYSH | 1.8.99.— | E |
| | | H2SO3 + PAP | | | |
| 3′-Phospho-adenylylsulfate reductase | yitB | PAPS + RTHIO -> OTHIO + | CYSH2 | 1.8.99.— | |
| | | H2SO3 + PAP | | | |
| Adenylylsulfate kinase | cysC | APS + ATP -> ADP + PAPS | CYSC | 2.7.1.25 | E |
| Adenylylsulfate kinase | yisZ | APS + ATP -> ADP + PAPS | CYSC2 | 2.7.1.25 | |
| O-Acetylserine (thiol)-lyase A | cysK | ASER + H2S -> AC + CYS | CYSK | 4.2.99.8 | E |
| O-Acetylserine (thiol)-lyase B | ytkP | ASER + H2S -> AC + CYS | CYSM | 4.2.99.8 | |
| O-Acetylserine (thiol)-lyase B | yrhA | ASER + H2S -> AC + CYS | CYSM2 | 4.2.99.8 | |
| Serine transacetylase | cysE | SER + ACCOA <-> COA + ASER | CYSE | 2.3.1.30 | E |
| Sulfate adenylyltransferase | sat | H2SO4 + ATP + GTP -> PPI + APS + | CYSD | 2.7.7.4 | E |
| | | GDP + PI | | | |
| Sulfate adenylyltransferase | yitA | H2SO4 + ATP + GTP -> PPI + APS + | CYSD2 | 2.7.7.4 | |
| | | GDP + PI | | | |
| Sulfite reductase | yvgQ | H2SO3 + 3 NADPH <-> H2S + 3 NADP | CYSI | 1.8.1.2 | E |
| Sulfite reductase | yvgR_{—} | H2SO3 + 3 NADPH <-> H2S + 3 NADP | CYSI2 | 1.8.1.2 | |
| Threonine and Lysine Biosynthesis | | | | | |
| Aspartate kinase I | dapG | ASP + ATP <-> ADP + BASP | THRA1 | 2.7.2.4 | E |
| Aspartate kinase II | lysC | ASP + ATP <-> ADP + BASP | METL1 | 2.7.2.4 | |
| Aspartate kinase III | yclM | ASP + ATP <-> ADP + BASP | LYSC | 2.7.2.4 | |
| Aspartate semialdehyde | asd | BASP + NADPH <-> NADP + | ASD | 1.2.1.11 | E |
| dehydrogenase | | PI + ASPSA | | | |
| Diaminopimelate decarboxylase | lysA | MDAP -> CO2 + LYS | LYSA | 4.1.1.20 | E |
| Diaminopimelate epimerase | dapF | D26PIM <-> MDAP | DAPF | 5.1.1.7 | E |
| Dihydrodipicolinate reductase | dapB | D23PIC + NADPH -> NADP + PIP26DX | DAPB | 1.3.1.26 | E |
| Dihydrodipicolinate synthase | dapA | ASPSA + PYR -> D23PIC | DAPA | 4.2.1.52 | E |
| Homoserine dehydrogenase I | hom | ASPSA + NADPH <-> NADP + HSER | THRA2 | 1.1.1.3 | E |
| Homoserine kinase | thrB | HSER + ATP -> ADP + PHSER | THRB | 2.7.1.39 | E |
| Lysine decarboxylase 1 | yaaO | LYS -> CO2 + CADV | CADA | 4.1.1.18 | |
| Succinyl diaminopimelate | DAPC | NS2A6O + GLU <-> AKG + NS26DP | DAPC | 2.6.1.17 | E |
| aminotransferase | | | | | |
| Succinyl diaminopimelate | yodQ | NS26DP -> SUCC + D26PIM | DAPE | 3.5.1.18 | E |
| desuccinylase | | | | | |
| Tetrahydrodipicolinate succinylase | ykuQ | PIP26DX + SUCCOA -> | DAPD | 2.3.1.117 | E |
| | | COA + NS2A6O | | | |
| Threonine synthase | thrC | PHSER -> PI + THR | THRC1 | 4.2.99.2 | E |
| Methionine Biosynthesis | | | | | |
| Adenosyl homocysteinase (Unknown) | Deduction | HCYS + ADN <-> SAH | ADCSASE | 3.3.1.1 | E |
| Cobalamin-dependent methionine | metE | HCYS + MTHF -> MET +THF | METH | 2.1.1.13 | E |
| synthase | | | | | |
| Cystathionine- -lyase | yjcJ | LLCT -> HCYS + PYR + NH3 | METC | 4.4.1.8 | |
| Homoserine transsuccinylase | metA | HSER + SUCCOA -> COA + OSLHSER | META | 2.3.1.46 | E |
| O-succinlyhomoserine lyase | yjcI1 | OSLHSER + CYS -> SUCC + LLCT | METB1a | 4.2.99.9 | |
| O-succinlyhomoserine lyase | yrhB1 | OSLHSER + CYS -> SUCC + LLCT | METB1b | 4.2.99.9 | |
| O-Succinlyhomoserine lyase | yjcI2 | OSLHSER + H2S -> SUCC + HCYS | METB3a | | |
| O-Succinlyhomoserine lyase | yrhB2 | OSLHSER + H2S -> SUCC + HCYS | METB3b | | |
| O-Succinlyhomoserine lyase | yjcI3 | OSLHSER + CH3SH -> SUCC + MET | METB4a | | |
| O-Succinlyhomoserine lyase | yrhB3 | OSLHSER + CH3SH -> SUCC + MET | METB4b | | |
| S-Adenosylmethionine synthetase | metK | MET + ATP -> PPI + PI + SAM | METK | 2.5.1.6 | E |
| Amino Acid Degradation | | | | | |
| Alanine | | | | | |
| Alanine dehydrogenase | ald | ALA + NAD -> PYR + NH3 + NADH | ALD | 1.4.1.1 | |
| Arginine | | | | | |
| Arginase | rocF | ARG -> ORN + UREA | ROCF | 3.5.3.1 | |
| Aminobutyrate aminotransaminase 1 | gabT | GABA + AKG -> SUCCSAL + GLU | GABT | 2.6.1.19 | |
| Succinate semialdehyde | gabD | SUCCSAL + NAD -> SUCC + NADH | SAD | 1.2.1.16 | |
| dehydrogenase-NAD | | | | | |
| Asparagine | | | | | |
| Asparininase I | ansA | ASN -> ASP + NH3 | ASNA2 | 3.5.1.1 | |
| Asparininase II | yccC | ASN -> ASP + NH3 | ASNB2 | 3.5.1.1 | |
| Aspartate | | | | | |
| Aspartate ammonia-lyase | ansB | ASP -> FUM + NH3 | ASPA | 4.3.1.1 | |
| Glutamine | | | | | |
| Glutaminase A | ybgJ | GLN -> GLU + NH3 | GLNASEA | 3.5.1.2 | |
| Glutaminase B | ylaM | GLN -> GLU + NH3 | GLNASEB | 3.5.1.2 | |
| Histidine | | | | | |
| Formiminoglutamate hydrolase | hutG | NFGLU -> GLU + FAM | HUTG | 3.5.3.8 | |
| Histidase | hutH | HIS -> URCAN + NH3 | HUTH | 4.3.1.3 | |
| Imidazolone-5-propionate hydrolase | hutI | 4IMZP -> NFGLU | HUTI | 3.5.2.7 | |
| Urocanase | hutU | URCAN -> 4IMZP | HUTU | 4.2.1.49 | |
| Formamidase | FORAMD | FAM -> NH3 + FOR | FORAMD | | |
| Isoleucine, Leucine, Valine | | | | | |
| Leucine dehydrogenase | bcd | LEU + NAD -> OICAP + NH3 + NADH | LEUDH | | |
| Acyl-CoA dehydrogenase | mmgC1 | 3MBCOA + NADP -> | ACYLCOA1 | 1.3.99.3 | |
| | | 3M2ECOA + NADPH | | | |
| Acyl-CoA dehydrogenase | mmgC2 | ISBCOA + NADP -> MCCOA + NADPH | ACYLCOA2 | 1.3.99.3 | |
| Acyl-CoA dehydrogenase | mmgC3 | 2MBCOA + NADP -> | ACYLCOA3 | 1.3.99.3 | |
| | | 2MBECOA + NADPH | | | |
| Methylcrotonoyl-CoA carboxylase | MCCOAC | 3M2ECOA + ATP + CO2 -> 3MGCOA + | MCCOAC | 6.4.1.4 | |
| | | PI + ADP | | | |
| Methylglutaconyl-CoA hydratase | MGCOAH | 3MGCOA -> 3HMGCOA | MGCOAH | 4.2.1.18 | |
| Hydroxymethylglutaryl-CoA lyase | yngG | 3HMGCOA -> ACCOA + AAC | HMGCOAL | 4.1.3.4 | |
| succinyl CoA:3-oxoacid CoA- | scoA | SUCCOA + AAC -> SUCC + AACCOA | SUCCOAT | | |
| transferase | | | | | |
| branched-chain alpha-keto acid | bkdAA1 | OICAP + COA + NAD -> 3MBCOA + | BKDAA1 | | E |
| dehydrogenase | | CO2 + NADH | | | |
| branched-chain alpha-keto acid | bkdAA2 | OIVAL + COA + NAD -> ISBCOA + | BKDAA2 | | E |
| dehydrogenase | | CO2 + NADH | | | |
| branched-chain alpha-keto acid | bkdAA3 | OMVAL + COA + NAD -> 2MBCOA + | BKDAA3 | | E |
| dehydrogenase | | CO2 + NADH | | | |
| 3-hydroxbutyryl-CoA dehydratase | yngF1 | MCCOA -> 3HIBCOA | yngF1 | 4.2.1.17 | |
| 3-hydroxbutyryl-CoA dehydratase | ysiB1 | MCCOA -> 3HIBCOA | ysiB1 | 4.2.1.17 | |
| 3-hydroxbutyryl-CoA dehydratase | yngF2 | 2MBECOA -> 3H2MBCOA | yngF2 | 4.2.1.17 | |
| 3-hydroxbutyryl-CoA dehydratase | ysiB2 | 2MBECOA -> 3H2MBCOA | ysiB2 | 4.2.1.17 | |
| Acyl-CoA hydrolase | ykhA | 3HIBCOA -> 3H2MP + COA | ykhA | 3.1.2.4 | |
| Methylmalonyl-CoA epimerase | MMCOAEP | SMMCOA <-> | MMCOAEP | 5.1.99.1 | |
| (5.1.99.1) | | RMMCOA | | | |
| Methylmalonyl-CoA mutase (5.4.99.2) | MMCOAMT | RMMCOA -> SUCCOA | MMCOAMT | 5.4.99.2 | |
| Proline | | | | | |
| Pyrroline-5-carboxylate dehydrogenase | rocA | GLUGSAL + NAD -> NADH + GLU | PUTA2 | 1.5.1.12 | |
| Proline oxidase | ycgM | PRO + NADP -> GLUGSAL + NADPH | PROOX | 1.5.1.2 | |
| Serine | | | | | |
| D-Serine deaminase | dsdA | DSER -> PYR + NH3 | DSDA | 4.2.1.14 | |
| Serine deaminase 1 | sdaAA | SER -> PYR + NH3 | SDAA1 | 4.2.1.13 | |
| Threonine | | | | | |
| Amino ketobutyrate ligase | kbl | 2A3O + COA -> ACCOA + GLY | KBL2 | 2.3.1.29 | |
| Threonine dehydrogenase | tdh | THR + NAD -> 2A3O + NADH | TDH2 | 1.1.1.103 | |
| Purine Biosynthesis | | | | | |
| 5′-Phosphoribosyl-4-(N- | purB2 | SAICAR <-> FUM + AICAR | PURB1 | 4.3.2.2 | E |
| succinocarboxamide)-5-aminoimidazole | | | | | |
| lyase | | | | | |
| Adenylosuccinate lyase | purB1 | ASUC <-> FUM + AMP | PURB2 | 4.3.2.2 | E |
| Adenylosuccinate synthetase | purA | IMP + GTP + ASP -> GDP + PI + ASUC | PURA | 6.3.4.4 | E |
| AICAR transformylase | purH1 | AICAR + FTHF <-> THF + PRFICA | PURH1 | 2.1.2.3 | E |
| Amidophosphoribosyl transferase | purF | PRPP + GLN -> PPI + GLU + PRAM | PURF | 2.4.2.14 | E |
| GMP reductase | guaC | GMP + NADPH -> NADP + IMP + NH3 | GUAC | 1.6.6.8 | |
| GMP synthase | guaA | XMP + ATP + GLN -> GLU + AMP + | GUAA | 6.3.4.1 | E |
| | | PPI + GMP | | | |
| IMP cyclohydrolase | purH2 | PRFICA <-> IMP | PURH2 | 3.5.4.10 | E |
| IMP dehydrogenase | guaB | IMP + NAD -> NADH + XMP | GUAB | 1.1.1.205 | E |
| IMP dehydrogenase | yhcV | IMP + NAD -> NADH + XMP | GUAB2 | 1.1.1.205 | |
| IMP dehydrogenase | ylbB | IMP + NAD -> NADH + XMP | GUAB3 | 1.1.1.205 | |
| Phosphoribosylamine-glycine ligase | purD | PRAM + ATP + GLY <-> | PURD | 6.3.4.13 | E |
| | | ADP + PI + GAR | | | |
| Phosphoribosylaminoimidazole | purE | AIR + CO2 + ATP <-> | PURK | 4.1.1.21 | E |
| carboxylase 1 | | NCAIR + ADP + PI | | | |
| Phosphoribosylaminoimidazole | purK | NCAIR + CO2 <-> CAIR | PURE | 4.1.1.21 | E |
| carboxylase 2 | | | | | |
| Phosphoribosylaminoimidazole- | purC | CAIR + ATP + ASP <-> | PURC | 6.3.2.6 | E |
| succinocarboxamide synthetase | | ADP + PI + SAICAR | | | |
| Phosphoribosylformylglycinamide | purM | FGAM + ATP -> ADP + PI + AIR | PURM | 6.3.3.1 | E |
| cyclo-ligase | | | | | |
| Phosphoribosylformylglycinamide | purL | FGAR + ATP + GLN -> GLU + ADP + | PURL | 6.3.5.3 | E |
| synthetase | | PI + FGAM | | | |
| Phosphoribosylformylglycinamide | purQ | FGAR + ATP + GLN -> GLU + ADP + | PURL2 | 6.3.5.3 | |
| synthetase | | PI + FGAM | | | |
| Phosphoribosylglycinamide | purN | GAR + FTHF <-> THF + FGAR | PURN | 2.1.2.2 | E |
| formyltransferase | | | | | |
| Phosphoribosylglycinamide | purT | GAR + FTHF <->THF + FGAR | PURN2 | 2.1.2.2 | |
| formyltransferase | | | | | |
| Pyrimidine Biosynthesis | | | | | |
| Aspartate-carbamoyltransferase | pyrB | CAP + ASP -> CAASP + PI | PYRB | 2.1.3.2 | E |
| CTP synthetase | pyrG1 | UTP + GLN + ATP -> GLU + CTP + | PYRG1 | 6.3.4.2 | |
| | | ADP + PI | | | |
| CTP synthetase | pyrG2 | UTP + NH3 + ATP -> CTP + ADP + PI | PYRG2 | | |
| Dihydroorotase | pyrC | CAASP <-> DOROA | PYRC | 3.5.2.3 | E |
| Dihydroorotate dehydrogenase | pyrD | DOROA + Q <-> QH2 + OROA | PYRD | 1.3.3.1 | E |
| OMP decarboxylase | pyrF | OMP -> CO2 + UMP | PYRF | 4.1.1.23 | E |
| Orotate phosphoribosyl transferase | pyrE | OROA + PRPP <-> PPI + OMP | PYRE | 2.4.2.10 | E |
| Salvage Pathways | | | | | |
| 5′-Nucleotidase | yhcR1 | DUMP -> DU + PI | USHA1 | 3.1.3.5 | |
| 5′-Nucleotidase | yhcR4 | DTMP -> DT + PI | USHA2 | 3.1.3.5 | |
| 5′-Nucleotidase | yhcR5 | DAMP -> DA + PI | USHA3 | 3.1.3.5 | |
| 5′-Nucleotidase | yhcR6 | DGMP -> DG + PI | USHA4 | 3.1.3.5 | |
| 5′-Nucleotidase | yhcR7 | DCMP -> DC + PI | USHA5 | 3.1.3.5 | |
| 5′-Nucleotidase | yhcR8 | CMP -> CYTD + PI | USHA6 | 3.1.3.5 | |
| 5′-Nucleotidase | yhcR9 | AMP -> PI + ADN | USHA7 | 3.1.3.5 | |
| 5′-Nucleotidase | yhcR10 | GMP -> PI + GSN | USHA8 | 3.1.3.5 | |
| 5′-Nucleotidase | yhcR11 | IMP -> PI + INS | USHA9 | 3.1.3.5 | |
| 5′-Nucleotidase | yhcR3 | XMP -> PI + XTSN | USHA12 | 3.1.3.5 | |
| 5′-Nucleotidase | yhcR2 | UMP -> PI + URI | USHA11 | 3.1.3.5 | |
| Adenine deaminase | adeC | AD -> NH3 + HYXN | YICP | 3.5.4.2 | |
| Adenine deaminase | yerA | AD -> NH3 + HYXN | YICP2 | 3.5.4.2 | |
| Adenine phosphoryltransferase | apt | AD + PRPP -> PPI + AMP | APT | 2.4.2.7 | |
| Adenosine kinase | adk1 | ADN + ATP -> AMP + ADP | ADKIN | 2.7.1.20 | |
| Adenylate kinase | adk2 | ATP + AMP <-> 2 ADP | ADK1 | 2.7.4.3 | |
| Adenylate kinase | adk3 | GTP + AMP <-> ADP + GDP | ADK2 | 2.7.4.3 | |
| Adenylate kinase | adk4 | ITP + AMP <-> ADP + IDP | ADK3 | 2.7.4.3 | |
| Adenylate kinase | adk5 | DAMP + ATP <-> ADP + DADP | ADK4 | 2.7.4.11 | |
| Cytidine deaminase | cdd1 | CYTD -> URI + NH3 | CDD1 | 3.5.4.5 | |
| Cytidine deaminase | cdd2 | DC -> NH3 + DU | CDD2 | 3.5.4.5 | |
| Cytidylate kinase | cmk1 | DCMP + ATP <-> ADP + DCDP | CMKA1 | 2.7.4.14 | |
| Cytidylate kinase | cmk2 | CMP + ATP <-> ADP + CDP | CMKA2 | 2.7.4.14 | |
| Cytidylate kinase | cmk3 | UMP + ATP <-> ADP + UDP | CMKA3 | 2.7.4.14 | |
| Cytodine kinase | udk5 | CYTD + GTP -> GDP + CMP | UDK2 | 2.7.1.48 | |
| Deoxyguanylate kinase | gmk2 | DGMP + ATP <-> DGDP + ADP | GMK2 | 2.7.4.8 | |
| dTMP kinase | tmk | DTMP + ATP <-> ADP + DTDP | TMK | 2.7.4.9 | E |
| dUTP pyrophosphatase | yncF | DUTP -> PPI + DUMP | DUT | 3.6.1.23 | |
| dUTP pyrophosphatase | yosS | DUTP -> PPI + DUMP | DUT2 | 3.6.1.23 | |
| Guanylate kinase | gmk1 | GMP + ATP <-> GDP + ADP | GMK1 | 2.7.4.8 | |
| Nucleoside triphosphatase | phoA2 | GTP -> GSN + 3 PI | MUTT1 | 3.6.1.— | |
| Nucleoside triphosphatase | phoB2 | GTP -> GSN + 3 PI | MUTT1b | 3.6.1.— | |
| Nucleoside triphosphatase | phoA3 | DGTP -> DG + 3 PI | MUTT2 | 3.6.1.— | |
| Nucleoside triphosphatase | phoB3 | DGTP -> DG + 3 PI | MUTT2b | 3.6.1.— | |
| Nucleoside-diphosphate kinase | ndk2 | GDP + ATP <-> GTP + ADP | NDK1 | 2.7.4.6 | |
| Nucleoside-diphosphate kinase | ndk3 | UDP + ATP <-> UTP + ADP | NDK2 | 2.7.4.6 | E |
| Nucleoside-diphosphate kinase | ndk4 | CDP + ATP <-> CTP + ADP | NDK3 | 2.7.4.6 | |
| Nucleoside-diphosphate kinase | ndk5 | DGDP + ATP <-> DGTP + ADP | NDK5 | 2.7.4.6 | |
| Nucleoside-diphosphate kinase | ndk6 | DUDP + ATP <-> DUTP + ADP | NDK6 | 2.7.4.6 | |
| Nucleoside-diphosphate kinase | ndk7 | DCDP + ATP <-> DCTP + ADP | NDK7 | 2.7.4.6 | |
| Nucleoside-diphosphate kinase | ndk8 | DADP + ATP <-> DATP + ADP | NDK9 | 2.7.4.6 | |
| Nucleoside-diphosphate kinase | ndk1 | DTDP + ATP <-> DTTP + ADP | NDK0 | 2.7.4.6 | E |
| Purine nucleotide phosphorylase | deoD1 | DIN + PI <-> HYXN + DR1P | DEOD1 | 2.4.2.1 | |
| Purine nucleotide phosphorylase | punA1 | DIN + PI <-> HYXN + DR1P | PUNA1 | 2.4.2.1 | |
| Purine nucleotide phosphorylase | deoD2 | DA + PI <-> AD + DR1P | DEOD2 | 2.4.2.1 | |
| Purine nucleotide phosphorylase | punA2 | DA + PI <-> AD + DR1P | PUNA2 | 2.4.2.1 | |
| Purine nucleotide phosphorylase | deoD3 | DG + PI <-> GN + DR1P | DEOD3 | 2.4.2.1 | |
| Purine nucleotide phosphorylase | punA3 | DG + PI <-> GN + DR1P | PUNA3 | 2.4.2.1 | |
| Purine nucleotide phosphorylase | deoD4 | HYXN + R1P <-> INS + PI | DEOD4 | 2.4.2.1 | |
| Purine nucleotide phosphorylase | punA4 | HYXN + R1P <-> INS + PI | PUNA4 | 2.4.2.1 | |
| Purine nucleotide phosphorylase | deoD5 | AD + R1P <-> PI + ADN | DEOD5 | 2.4.2.1 | |
| Purine nucleotide phosphorylase | punA5 | AD + R1P <-> PI + ADN | PUNA5 | 2.4.2.1 | |
| Purine nucleotide phosphorylase | deoD6 | GN + R1P <-> PI + GSN | DEOD6 | 2.4.2.1 | |
| Purine nucleotide phosphorylase | punA6 | GN + R1P <-> PI + GSN | PUNA6 | 2.4.2.1 | |
| Purine nucleotide phosphorylase | deoD7 | XAN + R1P <-> PI + XTSN | DEOD7 | 2.4.2.1 | |
| Purine nucleotide phosphorylase | punA7 | XAN + R1P <-> PI + XTSN | PUNA7 | 2.4.2.1 | |
| Purine nucleotide phosphorylase | deoD8 | DU + PI <-> URA + DR1P | DEOD8 | 2.4.2.1 | |
| Purine nucleotide phosphorylase | punA8 | DU + PI <-> URA + DR1P | PUNA8 | 2.4.2.1 | |
| Ribonucleoside-diphosphate reductase | nrdE1 | ADP + RTHIO -> DADP + OTHIO | NRDA1 | 1.17.4.1 | |
| Ribonucleoside-diphosphate reductase | nrdE2 | GDP + RTHIO -> DGDP + OTHIO | NRDA2 | 1.17.4.1 | |
| Ribonucleoside-diphosphate reductase | nrdE3 | CDP + RTHIO -> DCDP + OTHIO | NRDA3 | 1.17.4.1 | |
| Ribonucleoside-diphosphate reductase | nrdE4 | UDP + RTHIO -> DUDP + OTHIO | NRDA4 | 1.17.4.1 | |
| Ribonucleoside-triphosphate reductase | nrdE5 | ATP + RTHIO -> DATP + OTHIO | NRDD1 | 1.17.4.2 | |
| Ribonucleoside-triphosphate reductase | nrdE6 | GTP + RTHIO -> DGTP + OTHIO | NRDD2 | 1.17.4.2 | |
| Ribonucleoside-triphosphate reductase | nrdE7 | CTP + RTHIO -> DCTP + OTHIO | NRDD3 | 1.17.4.2 | |
| Ribonucleoside-triphosphate reductase | nrdE8 | UTP + RTHIO -> OTHIO + DUTP | NRDD4 | 1.17.4.2 | |
| Ribonucleoside-triphosphate reductase | yosNP1 | ADP + RTHIO -> DADP + OTHIO | yosNP1 | 1.17.4.2 | |
| Ribonucleoside-triphosphate reductase | yosNP2 | GDP + RTHIO -> DGDP + OTHIO | yosNP2 | 1.17.4.2 | |
| Ribonucleoside-triphosphate reductase | yosNP3 | CDP + RTHIO -> DCDP + OTHIO | yosNP3 | 1.17.4.2 | |
| Ribonucleoside-triphosphate reductase | yosNP4 | UDP + RTHIO -> DUDP + OTHIO | yosNP4 | 1.17.4.2 | |
| Ribonucleoside-triphosphate reductase | yosNP5 | ATP + RTHIO -> DATP + OTHIO | yosNP5 | 1.17.4.2 | |
| Ribonucleoside-triphosphate reductase | yosNP6 | GTP + RTHIO -> DGTP + OTHIO | yosNP6 | 1.17.4.2 | |
| Ribonucleoside-triphosphate reductase | yosNP7 | CTP + RTHIO -> DCTP + OTHIO | yosNP7 | 1.17.4.2 | |
| Ribonucleoside-triphosphate reductase | yosNP8 | UTP + RTHIO -> OTHIO + DUTP | yosNP8 | 1.17.4.2 | |
| Thymidilate synthetase | thyA | DUMP + METTHF -> DHF + DTMP | THYA | 2.1.1.45 | E |
| Thymidilate synthetase | thyB | DUMP + METTHF -> DHF + DTMP | THYA2 | 2.1.1.45 | |
| Thymidine (deoxyuridine) kinase | tdk1 | DU + ATP -> DUMP + ADP | TDK1 | 2.7.1.21 | |
| Thymidine (deoxyuridine) kinase | tdk2 | DT + ATP -> ADP + DTMP | TDK2 | 2.7.1.21 | |
| Thymidine (deoxyuridine) | deoD9 | DT + PI <-> THY + DR1P | DEOA2 | 2.4.2.4 | |
| phosphorylase | | | | | |
| Thymidine (deoxyuridine) | punA9 | DT + PI <-> THY + DR1P | PUNA9 | 2.4.2.4 | |
| phosphorylase | | | | | |
| Uracil phosphoribosyltransferase | upp | URA + PRPP -> UMP + PPI | UPP | 2.4.2.9 | |
| Uridine kinase | udk2 | URI + GTP -> GDP + UMP | UDK1 | 2.7.1.48 | |
| Uridylate kinase | pyrH1 | UMP + ATP <-> UDP + ADP | PYRH1 | 2.1.4.— | |
| Uridylate kinase | pyrH2 | DUMP + ATP <-> DUDP + ADP | PYRH2 | 2.1.4.— | |
| Xanthine-guanine | hrpT1 | XAN + PRPP -> XMP + PPI | GPT1 | 2.4.2.22 | |
| phosphoribosyltransferase | | | | | |
| Xanthine-guanine | hrpT2 | HYXN + PRPP -> PPI + IMP | GPT2 | 2.4.2.22 | |
| phosphoribosyltransferase | | | | | |
| Xanthine-guanine | hrpT3 | GN + PRPP -> PPI + GMP | GPT3 | 2.4.2.22 | |
| phosphoribosyltransferase | | | | | |
| One Carbon Metabolism | | | | | |
| Glycine cleavage system (Multi- | gcvPA | GLY + THF + NAD -> METTHF + | GCV | 1.4.4.2, | |
| component system) | | NADH + CO2 + NH3 | | 2.1.2.10 | |
| Formyl tetrahydrofolate deformylase | ykkE | FTHF -> FOR + THF | PURU | 3.5.1.10 | R |
| Methenyl tetrahydrofolate | folD2 | METHF <-> FTHF | FOLD2 | 3.5.4.9 | E |
| cyclehydrolase | | | | | |
| Methylene tetrahydrofolate reductase | METF | METTHF + NADH -> NAD + MTHF | METF | 1.7.99.5 | E |
| Methylene THF dehydrogenase | folD1 | METTHF + NADP <-> METHF + NADPH | FOLD1 | 1.5.1.5 | E |
| Membrane Lipid Biosynthesis | | | | | |
| Acetyl-CoA carboxyltransferase | accA | ACCOA + ATP + CO2 <-> MALCOA + | ACCA | 6.4.1.2, | E |
| | | ADP + PI | | 6.3.4.14 | |
| Acetyl-CoA-ACP transacylase | fabHAB0 | ACACP + COA <-> ACCOA + ACP | FABH | 2.3.1.41 | E |
| Isovaleryl-CoA ACP transacylase | 3MBACP | 3MBACP + COA <-> 3MBCOA + ACP | 3MBACP | | E |
| 2-Methylbutyryl-CoA ACP transacylase | 2MBACP | 2MBACP + COA <-> 2MBCOA + ACP | 2MBACP | | E |
| Isobutyryl-CoA ACP transacylase | ISBACP | ISBACP + COA <-> ISBCOA + ACP | ISBACP | | E |
| Acyltransferase | PLS2 | GL3P + 0.022 C140IACP + 0.046 | PLS2 | | E |
| | | C140NACP + 0.386 C150IACP + 0.654 | | | |
| | | C150AACP + 0.00001 C161IACP + 0.094 | | | |
| | | C160IACP + 0.00001 C161NACP + 0.202 | | | |
| | | C160NACP + 0.00001 C171IACP + | | | |
| | | 0.00001 C171AACP + 0.154 C170IACP + | | | |
| | | 0.362 C170AACP + 0.074 C180NACP -> | | | |
| | | 1.994 ACP + PA | | | |
| β-Ketoacyl-ACP synthase III | fabHAB1 | ISBACP + 5 MALACP + 10 NADPH -> 10 | fabHAB1 | | E |
| | | NADP + C140IACP + 5 CO2 + 5 ACP | | | |
| β-Ketoacyl-ACP synthase III | fabHAB2 | ACACP + 6 MALACP + 12 NADPH -> 12 | fabHAB2 | | E |
| | | NADP + C140NACP + 6 CO2 + 6 ACP | | | |
| β-Ketoacyl-ACP synthase III | fabHAB3 | 3MBACP + 5 MALACP + 10 NADPH -> | fabHAB3 | | E |
| | | 10 NADP + C150IACP + 5 CO2 + 5 ACP | | | |
| β-Ketoacyl-ACP synthase III | fabHAB4 | 2MBACP + 5 MALACP + 10 NADPH -> | fabHAB4 | | E |
| | | 10 NADP + C150AACP + 5 CO2 + 5 ACP | | | |
| β-Ketoacyl-ACP synthase III | fabHAB5 | ISBACP + 6 MALACP + 11 NADPH -> 11 | fabHAB5 | | E |
| | | NADP + C161IACP + 6 CO2 + 6 ACP | | | |
| β-Ketoacyl-ACP synthase III | fabHAB6 | ISBACP + 6 MALACP + 12 NADPH -> 12 | fabHAB6 | | E |
| | | NADP + C160IACP + 6 CO2 + 6 ACP | | | |
| β-Ketoacyl-ACP synthase III | fabHAB7 | ACACP + 7 MALACP + 13 NADPH -> 13 | fabHAB7 | | E |
| | | NADP + C161NACP + 7 CO2 + 7 ACP | | | |
| β-Ketoacyl-ACP synthase III | fabHAB8 | ACACP + 7 MALACP + 14 NADPH -> 14 | fabHAB8 | | E |
| | | NADP + C160NACP + 7 CO2 + 7 ACP | | | |
| β-Ketoacyl-ACP synthase III | fabHAB9 | 3MBACP + 6 MALACP + 11 NADPH -> | fabHAB9 | | E |
| | | 11 NADP + C171IACP + 6 CO2 + 6 ACP | | | |
| β-Ketoacyl-ACP synthase III | fabHAB10 | 2MBACP + 6 MALACP + 11 NADPH -> | fabHAB10 | | E |
| | | 11 NADP + C171AACP + 6 CO2 + 6 ACP | | | |
| β-Ketoacyl-ACP synthase III | fabHAB11 | 3MBACP + 6 MALACP + 12 NADPH -> | fabHAB11 | | E |
| | | 12 NADP + C170IACP + 6 CO2 + 6 ACP | | | |
| β-Ketoacyl-ACP synthase III | fabHAB12 | 2MBACP + 6 MALACP + 12 NADPH -> | fabHAB12 | | E |
| | | 12 NADP + C170AACP + 6 CO2 + 6 ACP | | | |
| β-Ketoacyl-ACP synthase III | fabHAB13 | ACACP + 8 MALACP + 16 NADPH -> 16 | fabHAB13 | | E |
| | | NADP + 1 C180NACP + 8 CO2 + 8 | | | |
| | | ACP | | | |
| Cardiolipin synthase | ywnE | 2 PG <-> CL + GL | CLS | 2.7.8.— | E |
| CDP-Diacylglycerol synthetase | cdsA | PA + CTP <-> CDPDG + PPI | CDSA | 2.7.7.41 | E |
| Malonyl-CoA-ACP transacylase | fabD | MALCOA + ACP <-> MALACP + COA | FADD1 | 2.3.1.39 | E |
| Phosphatidylglycerol phosphate | PGPA | PGP -> PI + PG | PGPA | 3.1.3.27 | E |
| phosphatase A | | | | | |
| Phosphatidylglycerol phosphate | pgsA | CDPDG + GL3P <-> CMP + PGP | PGSA | 2.7.8.5 | E |
| synthase | | | | | |
| Phosphatidylserine decarboxylase | psd | PS -> PE + CO2 | PSD | 4.1.1.65 | E |
| Phosphatidylserine synthase | pssA | CDPDG + SER <-> CMP + PS | PSSA | 2.7.8.8 | E |
| Fatty Acid Metabolism | | | | | |
| 3-Hydroxyacyl-CoA dehydrogenase | yusL1 | HACOA + NAD <-> OACOA + NADH | FADBS | 1.1.1.35 | |
| 3-Hydroxyacyl-CoA dehydrogenase | yusL2 | 3H2MBCOA + NAD -> | FADBS2 | 1.1.1.35 | |
| | | 2MAACOA + NADH | | | |
| 3-Ketoacyl-CoA thiolase | yusK1 | OACOA + COA -> ACOA + ACCOA | FADA | 2.3.1.16 | |
| 3-Ketoacyl-CoA thiolase | yusK2 | 2MAACOA + COA -> ACCOA + PPCOA | FADA2 | 2.3.1.16 | |
| Acetyl-CoA C-acetyltransferase | mmgA | 2 ACCOA <-> COA + AACCOA | ATOB | 2.3.1.9 | |
| Acyl-CoA dehydrogenase | acdA | ACOA + FAD -> 23DACOA + FADH | FADE | 1.3.99.3 | |
| Acyl-CoA synthetase | IcfA | ATP + LCCA + COA <-> AMP + | FADD | 6.2.1.3 | E |
| | | PPI + ACOA | | | |
| Isoprenoid Biosynthesis | | | | | |
| Farnesyl pyrophosphate synthetase | yqiD1 | DMPP + IPPP -> GPP + PPI | ISPA1 | 2.5.1.1 | E |
| Geranyltranstransferase | yqiD2 | GPP + IPPP -> FPP + PPI | ISPA2 | 2.5.1.10 | E |
| Isoprenyl pyrophosphate isomerase | ypgA | IPPP -> DMPP | IPPPISO | 5.3.3.2 | E |
| Isoprenyl-pyrophosphate synthesis | dxr_{—} | T3P1 + PYR + 2 NADPH + ATP -> | IPPPSYN | 8 rxns | E |
| pathway | | IPPP + ADP + 2 NADP + CO2 | | | |
| Octoprenyl pyrophosphate synthase (5 | ISPB | 5 IPPP + FPP -> OPP + 5 PPI | ISPB | 2.5.1.— | E |
| reactions) | | | | | |
| Undecaprenyl pyrophosphate synthase | UDPPSYN | 8 IPPP + FPP -> UDPP + 8 PPI | UDPPSYN | 2.5.1.31 | |
| (8 reactions) | | | | | |
| Quinone Biosynthesis | | | | | |
| Menaquinone | | | | | |
| Isochorismate synthase 1 | dhbC | CHOR -> ICHOR | MENF | 5.4.99.6 | E |
| Isochorismate synthase 1 | menF | CHOR -> ICHOR | MENF2 | 5.4.99.6 | |
| 1,4-Dihydroxy-2-naphthoate | menA | DHNA + OPP -> DMK + PPI + CO2 | MENA | 2.5.1.— | E |
| octaprenyltransferase | | | | | |
| α-Ketoglutarate decarboxylase | menD1 | AKG + TPP -> SSALTPP + CO2 | MEND1 | 4.1.1.71 | E |
| Naphthoate synthase | menB | OSBCOA -> DHNA + COA | MENB | 4.1.3.36 | E |
| O-Succinylbenzoate-CoA synthase | menC | SHCHC -> OSB | MENC | 4.2.1.— | E |
| O-Succinylbenzoic acid-CoA ligase | menE | OSB + ATP + COA -> OSBCOA + | MENE | 6.2.1.26 | E |
| | | AMP + PPI | | | |
| S-Adenosylmethionine-2-DMK | MENG | DMK + SAM -> Q + SAH | MENG | 2.1.1.— | E |
| methyltransferase | | | | | |
| SHCHC synthase | menD2 | ICHOR + SSALTPP -> PYR + | MEND2 | 4.1.3.— | E |
| | | TPP + SHCHC | | | |
| Enterochelin Biosynthesis | | | | | |
| 2,3-Dihydo-2,3-dihydroxybenzoate | dhbA | 23DHDHB + NAD <-> 23DHB + NADH | ENTA | 1.3.1.28 | |
| dehydrogenase | | | | | |
| ATP-dependent activation of 2,3- | dhbE | 23DHB + ATP <-> 23DHBA + PPI | ENTE | 6.—.—.— | |
| dihydroxybenzoate | | | | | |
| ATP-dependent serine activating | ENTF | SER + ATP <-> SERA + PPI | ENTF | 2.7.7.— | |
| enzyme | | | | | |
| Enterochelin synthetase | ENTD | 3 SERA + 3 23DHBA -> ENTER + 6 AMP | ENTD | 6.—.—.— | |
| Isochorismatase | dhbB | ICHOR <-> 23DHDHB + PYR | ENTB | 3.3.2.1 | |
| Riboflavin Biosynthesis | | | | | |
| 3,4 Dihydroxy-2-butanone-4-phosphate | ribA2 | RL5P -> DB4P + FOR | RIBB | 3.5.4.25 | E |
| synthase | | | | | |
| 6,7-Dimethyl-8-ribityllumazine synthase | ribA3 | DB4P + A6RP -> D8RL + PI | RIBE | 3.5.4.25 | E |
| FAD synthetase | ribC1 | FMN + ATP -> FAD + PPI | RIBF2 | 2.7.7.2 | E |
| GTP cyclohydrolase II | ribA1 | GTP -> D6RP5P + FOR + PPI | RIBA | 3.5.4.25 | E |
| Pryimidine deaminase | ribD | D6RP5P -> A6RP5P + NH3 | RIBD1 | 3.5.4.26 | E |
| Pyrimidine phosphatase | PMDPHT | A6RP5P2 -> A6RP + PI | PMDPHT | | E |
| Pyrimidine reductase | ribT | A6RP5P + NADPH -> A6RP5P2 + NADP | RIBD2 | 1.1.1.193 | E |
| Riboflavin kinase | ribC2 | RIBFLV + ATP -> FMN + ADP | RIBF1 | 2.7.1.26 | E |
| Riboflavin kinase | ribR_{—} | RIBFLV + ATP -> FMN + ADP | RIBF1b | | |
| Riboflavin synthase | ribE | 2 D8RL -> RIBFLV + A6RP | RIBC | 2.5.1.9 | E |
| Folate Biosynthesis | | | | | |
| 6-Hydroxymethyl-7,8 dihydropterin | folK | AHHMP + ATP -> AMP + AHHMD | FOLK | 2.7.6.3 | E |
| pyrophosphokinase | | | | | |
| Aminodeoxychorismate lyase | pabC | ADCHOR -> PYR + PABA | PABC | 4.—.—.— | E |
| Aminodeoxychorismate synthase | pabA1 | CHOR + GLN -> ADCHOR + GLU | PABA | 4.1.3.— | E |
| Dihydrofolate reductase | dfrA | DHF + NADPH -> NADP + THF | FOLA | 1.5.1.3 | E |
| Dihydrofolate synthetase | folC | DHPT + ATP + GLU -> ADP + PI + DHF | FOLC | 6.3.2.12 | E |
| Dihydroneopterin aldolase | folB | DHP -> AHHMP + GLAL | DHDNPA | 4.1.2.25 | E |
| Dihydropteroate synthase | sul | PABA + AHHMD -> PPI + DHPT | FOLP | 2.5.1.15 | E |
| GTP cyclohydrolase I | mtrA | GTP -> FOR + AHTD | FOLE | 3.5.4.16 | E |
| Nucleoside triphosphatase | phoA1 | AHTD -> DHP + 3 PI | MUTT | 3.1.3.1 | E |
| Nucleoside triphosphatase | phoB1 | AHTD -> DHP + 3 PI | MUTTa | 3.1.3.1 | |
| Coenzyme A Biosynthesis | | | | | |
| ACP Synthase | acpS | COA -> PAP + ACP | ACPS | 2.7.8.7 | |
| Aspartate decarboxylase | panD | ASP -> CO2 + bALA | PAND | 4.1.1.11 | E |
| DephosphoCoA kinase | ytaG | DPCOA + ATP -> ADP + COA | DPHCOAK | 2.7.1.24 | E |
| Ketopantoate reductase | ylbQ | AKP + NADPH -> NADP + PANT | PANE | 1.1.1.169 | E |
| Ketopentoate hydroxymethyl | panB | OIVAL + METTHF -> AKP + THF | PANB | 2.1.2.11 | E |
| transferase | | | | | |
| Pantoate-β-alanine ligase | panC | PANT + bALA + ATP -> AMP + | PANC | 6.3.2.1 | E |
| | | PPI + PNTO | | | |
| Pantothenate kinase | coaA | PNTO + ATP -> ADP + 4PPNTO | COAA | 2.7.1.33 | E |
| Phospho-pantethiene | PATRAN | 4PPNTE + ATP -> PPI + DPCOA | PATRAN | 2.7.7.3 | E |
| adenylyltransferase | | | | | |
| Phosphopantothenate-cysteine | PCDCL | 4PPNCYS -> CO2 + 4PPNTE | PCDCL | 4.1.1.36 | E |
| decarboxylase | | | | | |
| Phosphopantothenate-cysteine ligase | PCLIG | 4PPNTO + CTP + CYS -> CMP + | PCLIG | 6.3.2.5 | E |
| | | PPI + 4PPNCYS | | | |
| NAD Biosynthesis | | | | | |
| Aspartate oxidase | nadB | ASP + FAD -> FADH + ISUCC | NADB | 1.4.3.— | |
| Deamido-NAD ammonia ligase | nadE | NAAD + ATP + NH3 -> | NADE | 6.3.5.1 | |
| | | NAD + AMP + PPI | | | |
| NAD kinase | NADF | NAD + ATP -> NADP + ADP | NADF | 2.7.1.23 | |
| NADP phosphatase | NADPHPS | NADP -> NAD + PI | NADPHPS | 3.1.2.— | |
| NAMN adenylyl transferase | yqeJ1 | NAMN + ATP -> PPI + NAAD | NADD1 | 2.7.7.18 | |
| NAMN adenylyl transferase | yqeJ2 | NMN + ATP -> NAD + PPI | NADD2 | 2.7.7.18 | |
| Quinolate phosphoribosyl transferase | nadC | QA + PRPP -> NAMN + CO2 + PPI | NADC | 2.4.2.19 | |
| Quinolate synthase | nadA | ISUCC + T3P2 -> PI + QA | NADA | 1.4.3.16 | |
| PNC IV | | | | | |
| DNA ligase | ligA | NAD -> NMN + AMP | LIG | 6.5.1.2 | |
| Tetrapyrrole Biosynthesis | | | | | |
| 1,3-Dimethyluroporphyrinogen III | CYSG2 | PC2 + NAD -> NADH + SHCL | CYSG2 | | E |
| dehydrogenase | | | | | |
| Coproporphyrinogen oxidase, aerobic | hemN | O2 + CPP -> 2 CO2 + PPHG | HEMF | 1.3.3.3 | E |
| Coproporphyrinogen oxidase, aerobic | hemZ | O2 + CPP -> 2 CO2 + PPHG | HEMF2 | | |
| Ferrochelatase | hemH | PPIX -> PTH | HEHH | 4.99.1.1 | E |
| Glutamate-1-semialdehyde | gsaB | GSA -> ALAV | HEML | 5.4.3.8 | E |
| aminotransferase | | | | | |
| Glutamate-1-semialdehyde | hemL | GSA -> ALAV | HEML2 | | |
| aminotransferase | | | | | |
| Glutamyl-tRNA reductase | hemA | GTRNA + NADPH -> GSA + NADP | HEMA | 1.2.1.— | E |
| Glutamyl-tRNA synthetase | gltX | GLU + ATP -> GTRNA + AMP + PPI | GLTX | 6.1.1.17 | E |
| Heme O synthase | ctaO | PTH + FPP -> HO + PPI | CYOE | | E |
| Hydroxymethylbilane synthase | hemC | 4 PBG -> HMB + 4 NH3 | HEMC | 4.1.3.8 | E |
| Porphobilinogen synthase | hemB | 8 ALAV -> 4 PBG | HEMB | 4.2.1.24 | E |
| Protoporphyrinogen oxidase | hemY | O2 + PPHG -> PPIX | HEMG | 1.3.3.4 | E |
| Siroheme ferrochelatase | CYSG3 | SHCL -> SHEME | CYSG3 | 4.99.1.— | E |
| Uroporphyrin-III C-methyltransferase 1 | nasF | SAM + UPRG -> SAH + PC2 | HEMX | 2.1.1.107 | E |
| Uroporphyrin-III C-methyltransferase 2 | ylnD | SAM + UPRG -> SAH + PC2 | CYSG1 | 2.1.1.107 | |
| Uroporphyrin-III C-methyltransferase 2 | ylnF | SAM + UPRG -> SAH + PC2 | CYSG1a | | |
| Uroporphyrinogen decarboxylase | hemE | UPRG -> 4 CO2 + CPP | HEME | 4.1.1.37 | E |
| Uroporphyrinogen III synthase | hemD | HMB -> UPRG | HEMD | 4.2.1.75 | E |
| Heme A Synthase | ctaA | HO -> HEMEA | HEMAS | | E |
| Biotin Biosynthesis | | | | | |
| 8-Amino-7-oxononanoate synthase | bioF | ALA + CHCOA <-> CO2 + COA + AONA | BIOF | 2.3.1.47 | |
| Adenosylmethionine-8-amino-7- | bioA | SAM + AONA <-> SAMOB + DANNA | BIOA | 2.6.1.62 | |
| oxononanoate aminotransferase | | | | | |
| Adenosylmethionine-8-amino-7- | yodT | SAM + AONA <-> SAMOB + DANNA | BIOA2 | | |
| oxononanoate aminotransferase | | | | | |
| Biotin synthase | bioB | DTB + CYS <-> BT | BIOB | 2.8.1.— | |
| Dethiobiotin synthase | bioD | CO2 + DANNA + ATP <-> | BIOD | 6.3.3.3 | |
| | | DTB + PI + ADP | | | |
| Thiamin (Vitamin B1) Biosynthesis | | | | | |
| HMP kinase | HMPK | AHM + ATP -> AHMP + ADP | THIN | 2.7.1.49 | E |
| HMP-phosphate kinase | thiD | AHMP + ATP -> AHMPP + ADP | THID | 2.7.4.7 | E |
| Hypothetical Thimin Rxn | dxs | T3P1 + PYR -> DTP | UNKRXN1 | | E |
| Thiamin kinase | THIK | THMP + ADP <-> THIAMIN + ATP | THIK | 2.7.1.89 | E |
| Thiamin phosphate kinase | thiL | THMP + ATP <-> TPP + ADP | THIL | 2.7.4.16 | |
| Thiamin phosphate synthase | thiE | THZP + AHMPP -> THMP + PPI | THIB | 2.5.1.3 | E |
| thiC protein | thiC | AIR -> AHM | THIC | | E |
| thiF protein | thiF | DTP + TYR + CYS -> THZ + CO2 | THIFb | | E |
| thiG protein | thiG | DTP + TYR + CYS -> THZ + CO2 | THIGb | | |
| THZ kinase | thiM | THZ + ATP -> THZP + ADP | THIM | 2.7.1.50 | E |
| Cell Envelope Biosynthesis | | | | | |
| Glutamine fructose-6-phosphate | glmS | F6P + GLN -> GLU + GA6P | GLMS | 2.6.1.16 | E |
| Transaminase | | | | | |
| N-Acetylglucosamine-1-phosphate- | gcaD | UTP + GA1P + ACCOA -> UDPNAG + | GLMU | 2.7.7.23 | E |
| uridyltransferase | | PPI + COA | | | |
| Phosphoglucosamine mutase | GLMM | GA6P <-> GA1P | GLMM | | E |
| Techoic Acid Synthesis | | | | | |
| Techoic Acid Syn (TagA to O) | tagAO1 | PEPTIDO + UDPNAG + UDPNAMS + 30 | TASYN1 | | E |
| | | CDPGLYC + 10 UDPG + 10 DALA -> | | | |
| | | GLYTC1 + UMP + UDP + 30 CMP + 10 | | | |
| | | UDP | | | |
| Techoic Acid Syn (TagA to O) | tagAO2 | PEPTIDO + UDPNAG + UDPNAMS + 30 | TASYN2 | | E |
| | | UDPNAGAL + 30 UDPG -> GLYTC2 + | | | |
| | | UMP + UDP + 30 UMP + 30 UDP | | | |
| UDP-N-acetylglucosamine 2-epimerase | yvyH | UDPNAG <-> UDPNAMS | UDP2E | 5.1.3.14 | E |
| Glycerol-3-phosphate | tagD | CTP + T3P1 -> CDPGLYC + PPI | GLY3PCT | 2.7.7.39 | E |
| cytidylyltransferase | | | | | |
| Teichuronic Acid Synthesis | | | | | |
| Biosynthesis of teichuronic acid (UDP- | tuaD | UDPG -> UDPGCU | UDPGDH | | E |
| glucose 6-dehydrogenase) | | | | | |
| UDP-N-acetylglucosamine 4-epimerase | UDPNA4E | UDPNAG -> UDPNAGAL | UDPNA4E | | E |
| Teichuronic Acid Syn (tau A to H) | tuaAH | PEPTIDO + 30 UDPNAGAL + 30 | TUASYN | | E |
| | | UDPGCU -> 30 UMP + 30 UDP + TEICHU | | | |
| LPS sugar biosynthesis | | | | | |
| Diacylglycerol kinase | dgkA | DGR + ATP -> ADP + PA | DAGKIN | 2.7.1.107 | |
| Murein biosyntheis | | | | | |
| D-ala: D-ala ligases | ddl | 2 DALA + ATP <-> AA + ADP + PI | DDLA | 6.3.2.4 | E |
| D-Alanine-D-alanine adding enzyme | murF | UNAGD + ATP + AA -> UNAGDA + | MURF | 6.3.2.15 | E |
| | | ADP + PI | | | |
| Glutamate racemase | racE | GLU <-> DGLU | MURI | 5.1.1.3 | |
| Glutamate racemase | yrpC | GLU <-> DGLU | MURI2 | 5.1.1.3 | |
| N-Acetylglucosaminyl transferase | murG | UNPTDO + UDPNAG -> UDP + PEPTIDO | MURG | 2.7.8.13 | E |
| Phospho-N- | mraY | UNAGDA -> UMP + PI + UNPTDO | MRAY | 2.7.8.13 | E |
| acetylmuramoylpentapeptide | | | | | |
| transferase | | | | | |
| UDP-N-acetylglucosamine- | murB | UDPNAGEP + NADPH -> UDPNAM + | MURB | 1.1.1.158 | E |
| enolpyruvate dehydrogenase | | NADP | | | |
| UDP-N-acetylglucosamine- | murAA | UDPNAG + PEP -> UDPNAGEP + PI | MURA | 2.5.1.7 | E |
| enolpyruvate transferase | | | | | |
| UDP-N-acetylglucosamine- | murAB | UDPNAG + PEP -> UDPNAGEP + PI | MURA2 | 2.5.1.7 | |
| enolpyruvate transferase | | | | | |
| UDP-N-acetylmuramate-alanine ligase | murC | UDPNAM + ALA + ATP -> ADP + | MURC | 6.3.2.8 | E |
| | | PI + UDPNAMA | | | |
| UDP-N-acetylmuramoylalanine-D- | murD | UDPNAMA + DGLU + ATP -> | MURD | 6.3.2.9 | E |
| glutamate ligase | | UDPNAMAG + ADP + PI | | | |
| UDP-N-acetylmuramoylalanyl-D- | murE | UDPNAMAG + ATP + MDAP -> | MURE | 6.3.2.13 | E |
| glutamate 2,6-diaminopimelate ligase | | UNAGD + ADP + PI | | | |
| Membrane Transport | | | | | |
| Carbohydrates | | | | | |
| Arabinose (low affinity) | araE | ARABxt + HEXT <-> ARAB | ARABUP1 | | |
| Fructose | fruA | FRUxt + PEP -> F1P + PYR | FRUPTS | | |
| Fructose | levD | FRUxt + PEP -> F1P + PYR | FRUPTS2 | | |
| Glucitol | gutP | GLTxt + PEP -> GLT6P + PYR | GLTUP | | |
| Gluconate | gntP | GLCNxt + HEXT -> GLCN | GLCNUP2 | | |
| Gluconate | yojA | GLCNxt + HEXT -> GLCN | GLCNUP2 | | |
| Glucosamine | gamP | GLAMxt + PEP -> GA6P + PYR | GAUP | | |
| Glucose | ptsG | GLCxt + PEP -> G6P + PYR | GLCPTS | | E |
| Glycerol | glpF | GLxt <-> GL | GLUP | | |
| Maltose | malP | MLTxt + PEP -> MLT6P + PYR | MALUP1 | | |
| Mannitol | mtlA | MNTxt + PEP -> MNT6P + PYR | MNTUP | | |
| Mannose | manP | MANxt + PEP -> MAN1P + PYR | MANNUP | | |
| N-Acetylglucosamine | nagP | NAG + PEP -> NAGP + PYR | NAGUP | | |
| Ribose | rbsA | RIBxt + ATP -> RIB + ADP + PI | RIBUP | | |
| Sucrose | sacP | SUCxt + PEP -> SUC6P + PYR | SUCUP | | |
| Trehalose | treP | TRExt + PEP -> TRE6P + PYR | TREUP | | |
| Inositol | iolF | INOSTxt + HEXT -> INOSIT | INOSUP | | |
| Amino Acids | | | | | |
| Alanine | alsT | LAxt + HEXT -> ALA | ALAUP2 | | |
| Arginine | ARGUP | ARGxt + ATP -> ARG + ADP + PI | ARGUP | | |
| Arginine | ARGUP2 | ARGxt + HEXT <-> ARG | ARGUP2 | | |
| Asparagine (high Affinity) | ASNUP2 | ASNxt + ATP -> ASN + ADP + PI | ASNUP2 | | |
| Asparagine (low Affinity) | ASNUP1 | ASNxt + HEXT <-> ASN | ASNUP1 | | |
| Aspartate | ASPUP1 | ASPxt + HEXT -> ASP | ASPUP1 | | |
| Aspartate | ASPUP2 | ASPxt + ATP -> ASP + ADP + PI | ASPUP2 | | |
| Branched chain amino acid transport | BCAAUP1 | BCAAxt + HEXT <-> BCAA | BCAAUP1 | | |
| Dipeptide | dppB | DIPEPxt + ATP -> DIPEP + ADP + PI | DPEPUP | | |
| -Aminobutyrate transport | gabP | GABAxt + ATP -> GABA + ADP + PI | GABAUP | | |
| Glutamate | gltT | GLUxt + HEXT <-> GLU | GLUUP1 | | |
| Glutamate | gltP | GLUxt + HEXT <-> GLU | GLUUP2 | | |
| Glutamate | GLUUP3 | GLUxt + ATP -> GLU + ADP + PI | GLUUP3 | | |
| Glutamine | glnH | GLNxt + ATP -> GLN + ADP + PI | GLNUP | | |
| Histidine | ytmN | HISxt + ATP -> HIS + ADP + PI | HISUP | | |
| Histidine | hutM | HISxt + HEXT <-> HIS | HISUP2 | | |
| Isoleucine | ILEUP | ILExt + ATP -> ILE + ADP + PI | ILEUP | | |
| Leucine | LEUUP | LEUxt + ATP -> LEU + ADP + PI | LEUUP | | |
| Oligopeptide | appA | OPEPxt + ATP -> OPEP + ADP + PI | OPEPUP | | |
| Oligopeptide | oppA | OPEPxt + ATP -> OPEP + ADP + PI | OPEPUP2 | | |
| Ornithine | ORNUP | ORNxt + ATP -> ORN + ADP + PI | ORNUP | | |
| Ornithine | ORNUP2 | ORNxt + HEXT <-> ORN | ORNUP2 | | |
| Peptide | PEPUP | PEPTxt + ATP -> PEPT + ADP + PI | PEPUP | | |
| Phenlyalanine | PHEUP | PHExt + HEXT <-> PHE | PHEUP | | |
| Proline | opuE | PROxt + HEXT <-> PRO | PROUP | | |
| Proline | opuB2 | PROxt + ATP -> PRO + ADP + PI | PROUP2 | | |
| Threonine | THRUP1 | THRxt + ATP -> THR + ADP + PI | THRUP1 | | |
| Threonine | THRUP2 | THRxt + HEXT <-> THR | THRUP2 | | |
| Tyrosine | TYRUP | TYRxt + HEXT <-> TYR | TYRUP | | |
| Valine | VALUP | VALxt + ATP -> VAL + ADP + PI | VALUP | | |
| Purines & Pyrimidines | | | | | |
| Adenine | yxlA81 | ADxt + HEXT -> AD | ADUP | | |
| C-system | yxlA1 | ADNxt + HEXT -> ADN | NCCUP1 | | |
| C-system | nupC6 | URIxt + HEXT -> URI | NCCUP2 | | |
| C-system | nupC1 | CYTDxt + HEXT -> CYTD | NCCUP3 | | |
| C-system | nupC3 | DTxt + HEXT -> DT | NCCUP4 | | |
| C-system | yxlA2 | DAxt + HEXT -> DA | NCCUP5 | | |
| C-system | nupC2 | DCxt + HEXT -> DC | NCCUP6 | | |
| C-system | nupC4 | DUxt + HEXT -> DU | NCCUP7 | | |
| Cytosine | nupC7 | CYTSxt + HEXT -> CYTS | CYTSUP | | |
| G-system | yxlA5 | GSNxt + HEXT -> GSN | NCGUP2 | | |
| G-system | yxlA7 | XTSNxt + HEXT -> XTSN | NCGUP6 | | |
| G-system | yxlA3 | DGxt + HEXT -> DG | NCGUP9 | | |
| G-system (transports all nucleosides) | yxlA6 | INSxt + HEXT -> INS | NCGUP5 | | |
| Guanine | pbuG1 | GNxt <-> GN | GNUP | | |
| Hypoxanthine | pbuG2 | HYXNxt <-> HYXN | HYXNUP | | |
| Nucleosides and deoxynucleoside | yxlA4 | DINxt + HEXT -> DIN | NCUP8 | | |
| Uracil | pyrP | URAxt + HEXT -> URA | URAUP | | |
| Xanthine | pbuX | XANxt <-> XAN | XANUP | | |
| Metabolic By-Products | | | | | |
| Acetate transport | ACUP | ACxt + HEXT <-> AC | ACUP | | |
| Acetoin transport | ACTNUP | ACTNxt + HEXT <-> ACTN | ACTNUP | | |
| Diacetyl transport | DIACTUP | DIACTxt + HEXT <-> DIACT | DIACTUP | | |
| 2,3-Butanediol transport | BUTNUP | BUTNxt + HEXT <-> BUTN | BUTNUP | | |
| Ethanol transport | ETHUP | ETHxt + HEXT <-> ETH | ETHUP | | |
| Lactate transport | lctP | LACxt + HEXT <-> LAC | LACUP1 | | |
| Pyruvate transport | PYRUP | PYRxt + HEXT <-> PYR | PYRUP | | |
| Other Compounds | | | | | |
| α-Ketoglutarate | dctB5 | AKGxt + HEXT <-> AKG | AKGUP | | |
| α-Ketoglutarate/malate translocator | yflS | MALxt + AKG <-> MAL + AKGxt | AKMALUP | | |
| Ammonia transport | nrgA | NH3xt + HEXT <-> NH3 | NH3UP | | E |
| ATP drain flux for constant | ATPM | ATP -> ADP + PI | ATPM | | |
| maintanence requirements | | | | | |
| Carbon dioxide transport | CO2TX | CO2xt <-> CO2 | CO2TX | | E |
| Citrate | yraO | CITxt + HEXT -> CIT | CITUP | | |
| Dicarboxylates | dctB2 | SUCCxt + HEXT <-> SUCC | SUCCUP2 | | |
| Dicarboxylates | dctB1 | FUMxt + HEXT <-> FUM | FUMUP | | |
| Dicarboxylates | dctB3 | MALxt + HEXT <-> MAL | MALUP3 | | |
| Dicarboxylates | dctB4 | ASPxt + HEXT <-> ASP | ASPUP | | |
| Glycerol-3-phosphate | glpT | GL3Pxt + HEXT -> GL3P | GL3PUPa | | |
| Na/H antiporter | nhaC | NAxt + <-> NA + HEXT | NAUP1 | | |
| Na/H antiporter | mrpA | NAxt + <-> NA + HEXT | NAUP2 | | |
| Na/H antiporter | yhaU | NAxt + <-> NA + HEXT | NAUP3 | | |
| Na/H antiporter | yjbQ | NAxt + <-> NA + HEXT | NAUP4 | | |
| Nitrate transport | nasA | NO3xt + HEXT -> NO3 | NO3UP | | |
| Nitrate extrusion | narK | NO2xt + HEXT <-> NO2 | NO2UP | | |
| Nitrite transport | ywcJ | NO2xt + HEXT -> NO2 | NO2UP2 | | |
| Oxygen transport | O2TX | O2xt <-> O2 | O2TX | | E |
| Pantothenate | ywcA | PNTOxt + HEXT <-> PNTO | PANTOUP | | |
| Phosphate transport | pstA | PIxt + ATP -> ADP + 2 PI | PIUP1 | | |
| Phosphate transport | pit | PIxt + HEXT <-> PI | PIUP2 | | R |
| Potassium transport | trkA | Kxt + HEXT <-> K | POTUP2 | | |
| Sulfate transport | cysP | H2SO4xt + HEXT -> H2SO4 | H2SO4UP2 | | E |
| Urea transport | pucJ | UREAxt + 2 HEXT <-> UREA | UREATX | | |
| | FNADH | NAD -> NADH | FNADH | | |
| | FNADPH | NADP -> NADPH | FNADPH | | |
| | FATP | ADP + PI ->ATP | FATP | | |
| Miscellaneous Reactions | | | | | |
| beta-phosphoglucomutase/glucose-1- | pgcM | 2 G1P -> GLC + G16DP | BS001 | 5.4.2.6 | |
| phosphate phosphodismutase | | | | | |
| unknown; similar to 2′,3′-cyclic- | yfkN | 23CAMP -> 3AMP | BS002 | | |
| nucleotide 2°-phosphodiesterase | | | | | |
| 2-keto-3-deoxygluconate kinase | kdgK | 2D3D6PG + ATP -> ADP + 2KD6PG | BS003 | 2.7.1.45 | |
| 2-keto-3-deoxygluconate | kduD | 2DGLCN + NAD -> 3D2DGLCN + NADH | BS004 | 1.1.1.125 | |
| oxidoreductase | | | | | |
| methylmalonate-semialdehyde | mmsA | 2M3OP + COA + NAD -> PPCOA + | BS005 | 1.2.1.27 | |
| dehydrogenase | | CO2 + NADH | | | |
| unknown; similar to phosphoglycolate | yhcW | 2PGLYC + H2O -> GLYC + PI | BS006 | 3.1.3.18 | |
| phosphatase | | | | | |
| naringenin-chalcone synthase | bcsA | 3 MALCOA + CMRCOA -> 4 COA + | BS007 | 2.3.1.74 | |
| | | NARGC + 3 CO2 | | | |
| assimilatory nitrite reductase (subunit) | nasD | 3 NADPH + NO2 -> 3 NADP + NH3 | BS008 | 1.6.6.4 | |
| unknown; similar to 3- | yqeC | 3H2MP + NAD -> 2M3OP + NADH | BS009 | | |
| hydroxyisobutyrate dehydrogenase | | | | | |
| 3-hydroxybutyryl-CoA dehydrogenase | mmgB | 3HBCOA + NADP -> AACCOA + NADPH | | | |
| | BS010 | 1.1.1.157 | | | |
| 5-keto-4-deoxyuronate isomerase | kduI | 4D5HSUR <-> 3DG25DS | BS011 | 5.3.1.17 | |
| unknown; similar to 4- | yoaI | 4HPHAC + NADH + O2 -> 34DHPHAC + | BS012 | | |
| hydroxyphenylacetate-3-hydroxylase | | NAD | | | |
| unknown; similar to p-nitrophenyl | yutF | 4NPPI + H2O -> 4NPH + PI | BS013 | 3.1.3.41 | |
| phosphatase | | | | | |
| unknown; similar to 5-dehydro-4- | ycbC | 5D4DGLCR -> 25DXP + H2O + CO2 | BS014 | 4.2.1.41 | |
| deoxyglucarate dehydratase | | | | | |
| 6-phospho-beta-glucosidase | bglA | 6PGG -> GLC + G6P | BS015 | 3.2.1.86 | |
| 6-phospho-beta-glucosidase | licH | 6PGG -> GLC + G6P | BS016 | 3.2.1.86 | |
| unknown; similar to N- | yvcN | ACCOA + HXARA -> COA + ACARA | BS017 | | |
| hydroxyarylamine O-acetyltransferase | | | | | |
| probable maltose O-acetyltransferase | maa | ACCOA + MALT-> COA + ACMALT | BS018 | 2.3.1.79 | |
| unknown; similar to serine O- | yvfD | ACCOA + SER -> COA + OASER | BS019 | | |
| acetyltransferase | | | | | |
| alpha-acetolactate decarboxylase | alsD | ACLAC -> CO2 + ACTN | BS020 | 4.1.1.5 | |
| acetoin dehydrogenase E1 component | acoA | ACTN + NAD -> DIACT + NADH | BS021 | | |
| (TPP-dependent alpha subunit) | | | | | |
| acetoin dehydrogenase | acuA | ACTN + NAD -> DIACT + NADH | BS022 | | |
| Butanediol Dehydrogenase | BUTDH | ACTN + NADH <-> BUTN + NAD | BS023 | 1.1.1.4 | |
| ADP-ribose pyrophosphatase | nudF | ADPRIB -> R5P + AMP | BS024 | 3.6.1.13 | |
| unknown; similar to purine-cytosine | yxlA8 | ADxt + HEXT -> AD | BS025 | | |
| permease | | | | | |
| allantoinase | pucH | ALLTN -> ALLTT | BS026 | 3.5.2.5 | |
| tagaturonate reductase | uxaB | ALTRN + NAD -> TAGATU +NADH | BS027 | 1.1.1.58 | |
| unknown; similar to diadenosine | yjbP | APPPPA -> 2 ADP | BS028 | 3.6.1.41 | |
| tetraphosphatase | | | | | |
| probable branched-chain fatty-acid | buk | ATP + BUT -> ADP + BUTP | BS029 | 2.7.2.7 | |
| kinase (butyrate kinase) | | | | | |
| 6-carboxyhexanoate-CoA ligase | bioW | ATP + CHX -> AMP + PPI + CHCOA | BS030 | 6.2.1.14 | |
| deoxyadenosine/deoxycytidine kinase | dck1 | ATP + DA -> ADP + DAMP | BS031 | | |
| deoxyadenosine/deoxycytidine kinase | dck3 | ATP + DC -> ADP + DCMP | BS032 | | |
| deoxyadenosine/deoxycytidine kinase | dck2 | ATP + DG -> ADP + DGMP | BS033 | | |
| deoxyguanosine kinase | dgk1 | ATP + DG -> GDP + DAMP | BS034 | | |
| deoxyguanosine kinase | dgk2 | ATP + DIN -> IDP + DAMP | BS035 | | |
| unknown; similar to fructokinase | ydhR_{—} | ATP + FRUC -> ADP + F6P | BS036 | 2.7.1.4 | |
| unknown; similar to fructokinase | ydjE | ATP + FRUC -> ADP + F6P | BS037 | 2.7.1.4 | |
| GTP pyrophosphokinase (stringent | relA | ATP + GTP -> GDPTP + AMP | BS038 | 2.7.6.5 | |
| response) | | | | | |
| unknown; similar to GTP | yjbM | ATP + GTP -> GDPTP + AMP | BS039 | | |
| pyrophosphokinase | | | | | |
| unknown; similar to GTP- | ywaC | ATP + GTP -> GDPTP + AMP | BS040 | | |
| pyrophosphokinase | | | | | |
| unknown; similar to propionyl-CoA | yngE | ATP + PPCOA + CO2 -> ADP + | BS041 | 6.4.1.3 | |
| carboxylase | | PI + SMMCOA | | | |
| unknown; similar to propionyl-CoA | yqjD | ATP + PPCOA + CO2 -> ADP + | BS042 | | |
| carboxylase | | PI + SMMCOA | | | |
| unknown; similar to pyruvate, water | yvkC | ATP + PYR -> AMP + PEP + PI | BS043 | | |
| dikinase | | | | | |
| unknown; similar to benzaldehyde | yfmT | BENALD + NADP -> BENZ + NADPH | BS044 | | |
| dehydrogenase | | | | | |
| unknown; similar to aryl-alcohol | ycsN | BENOH + NAD-> BENALD + NADH | BS045 | 1.1.1.90 | |
| dehydrogenase | | | | | |
| probable phosphate butyryltransferase | ptb | BUTCOA + PI <-> COA + BUTP | BS046 | 2.3.1.19 | |
| unknown; similar to ribonucleoside- | yosN | CDP + RTHIO -> DCDP + OTHIO | BS047 | 1.17.4.1 | |
| diphosphate reductase (alpha subunit) | | | | | |
| unknown; similar to CDP-glucose 4,6- | yfnG | CDPGLC -> CDP46GLC + H2O | BS048 | 4.2.1.45 | |
| dehydratase | | | | | |
| choline ABC transporter (choline- | opuB3 | CHOLxt + ATP -> CHOL + ADP + PI | BS049 | | |
| binding protein) | | | | | |
| glycine betaine/carnitine/choline ABC | opuC2 | CHOLxt + ATP -> CHOL + ADP + PI | BS050 | | |
| transporter (membrane protein) | | | | | |
| para-aminobenzoate synthase | pabA2 | CHOR + GLN -> AN + PYR + GLU | BS051 | 4.1.3.— | |
| glutamine amidotransferase (subunit B)/ | | | | | |
| anthranilate synthase (subunit II) | | | | | |
| deoxyadenosine/deoxycytidine kinase | dck5 | CTP + DC -> CDP + DCMP | BS052 | | |
| unknown; similar to glucose-1- | yfnH | CTP + G1P -> PPI + CDPGLC | BS053 | 2.7.7.33 | |
| phosphate cytidylyltransferase | | | | | |
| unknown; similar to cysteine | yubC | CYS + O2 -> 3SALA | BS054 | | |
| dioxygenase | | | | | |
| uridine kinase | udk4 | CYTD + ATP -> ADP + CMP | BS055 | 2.7.1.48 | |
| uridine kinase | udk6 | CYTD + CTP -> CDP + CMP | BS056 | | |
| pyrimidine-nucleoside phosphorylase | pdp1 | CYTD + R1P -> CYTS + PI | BS057 | 2.4.2.2 | |
| probable D-alanine aminotransferase | dat | DALA + AKG -> PYR + DGLU | BS058 | 2.6.1.21 | |
| pyrimidine-nucleoside phosphorylase | pdp3 | DT + R1P-> THY + PI | BS059 | | |
| deoxyadenosine/deoxycytidine kinase | dck6 | DTTP + DC -> DTDP + DCMP | BS060 | | |
| alcohol dehydrogenase (assume | adhB | ETH + NAD -> ACAL + NADH | BS061 | 1.1.1.1; | |
| ethanol dehydrogenase) | | | | 1.2.1.1 | |
| NADP-dependent alcohol | adhA | ETH + NADP -> ACAL + NADPH | BS062 | 1.1.1.2 | |
| dehydrogenase | | | | | |
| unknown; similar to formaldehyde | yycR_{—} | FORMALD + NAD + H2O -> | BS063 | | |
| dehydrogenase | | FORMATE + NADH | | | |
| unknown; similar to formate transporter | yrhG | FORxt + HEXT <-> FOR | BS064 | | |
| glucuronate isomerase | uxaC2 | GALUR <-> FRCUR | BS065 | 5.3.1.12 | |
| glucose 1-dehydrogenase | gdh | GLC + NAD -> G15LAC + NADH | BS066 | 1.1.1.47 | |
| unknown; similar to glucose 1- | ycdF | GLC + NAD -> G15LAC + NADH | BS067 | | |
| dehydrogenase | | | | | |
| unknown; similar to glucose 1- | yhdF | GLC + NAD -> G15LAC + NADH | BS068 | | |
| dehydrogenase | | | | | |
| unknown; similar to glucose 1- | ykuF | GLC + NAD -> G15LAC + NADH | BS069 | | |
| dehydrogenase | | | | | |
| unknown; similar to glucose 1- | ykvO | GLC + NAD -> G15LAC + NADH | BS070 | | |
| dehydrogenase | | | | | |
| unknown; similar to glucose 1- | ywfD | GLC + NAD -> G15LAC + NADH | BS071 | | |
| dehydrogenase | | | | | |
| unknown; similar to glucose 1- | yxbG | GLC + NAD -> G15LAC + NADH | BS072 | | |
| dehydrogenase | | | | | |
| unknown; similar to glucose 1- | yxnA | GLC + NAD -> G15LAC + NADH | BS073 | | |
| dehydrogenase | | | | | |
| unknown; similar to gluconate 5- | yxjF | GLCN + NADP -> 5DHGLCN + NADPH | BS074 | 1.1.1.30 | |
| dehydrogenase | | | | | |
| unknown; similar to glycerate | yvcT | GLCR + NAD -> HPYR + NADH | BS075 | 1.1.1.215 | |
| dehydrogenase | | | | | |
| unknown; similar to glucarate | ycbF | GLCR -> 5D4DLCR + H2O | BS076 | 4.2.1.40 | |
| dehydratase | | | | | |
| glucuronate isomerase | uxaC1 | GLCUR <-> FRCUR | BS077 | 5.3.1.12 | |
| unknown; similar to glucosamine- | ybcM | GLN + F6P -> GLU + GLCAM6P | BS078 | 2.6.1.16 | |
| fructose-6-phosphate aminotransferase | | | | | |
| unknown; similar to glutamine-fructose- | yurP | GLN + F6P -> GLU + GLCAM6P | BS079 | | |
| 6-phosphate transaminase | | | | | |
| unknown; similar to 1-pyrroline-5- | ycgN | GLUGSAL + NAD -> GLU + NADH | BS080 | 1.5.1.12 | |
| carboxylate dehydrogenase | | | | | |
| unknown; similar to glycine oxidase | yurR_{—} | GLY + O2 -> GLX + NH3 + H2O2 | BS081 | | |
| glycine betaine ABC transporter (ATP- | opuA | GLYBETxt + ATP -> GLYBET + | BS082 | | |
| binding protein) | | ADP + PI | | | |
| choline ABC transporter (ATP-binding | opuB1 | GLYBETxt + ATP -> GLYBET + | BS083 | | |
| protein) | | ADP + PI | | | |
| glycine betaine/carnitine/choline ABC | opuC1 | GLYBETxt + ATP -> GLYBET + | BS084 | | |
| transporter (ATP-binding protein) | | ADP + PI | | | |
| glycerophosphoryl diester | glpQ | GLYPD + H2O -> ALC + GL3P | BS085 | 3.1.4.46 | |
| phosphodiesterase | | | | | |
| guanine deaminase | guaD | GN -> XAN + NH3 | BS086 | 3.5.4.3 | |
| deoxyadenosine/deoxycytidine kinase | dck4 | GTP + DC -> GDP + DCMP | BS087 | | |
| unknown; similar to carbonic anhydrase | ybcF | H2CO3 -> CO2 + H2O | BS088 | | |
| unknown; similar to carbonic anhydrase | ytiB | H2CO3 -> CO2 + H2O | BS089 | | |
| unknown; similar to carbonic anhydrase | yvdA | H2CO3 -> CO2 + H2O | BS090 | | |
| unknown; similar to epoxide hydrolase | yfhM | H2O + EPOX -> GLYCOL | BS091 | 3.3.2.3 | |
| unknown; similar to sulfite oxidase | yuiH | H2SO3 + O2 + H2O -> H2SO4 + H2O2 | BS092 | | |
| unknown; similar to hippurate hydrolase | ykuR_{—} | HIPP -> BENZ + GLY | BS093 | | |
| heptaprenyl diphosphate synthase | hepS | HXPP + IPPP -> PPI + HTPP | BS094 | | |
| component I | | | | | |
| unknown; similar to L-iditol 2- | ydjL | IDITOL + NAD -> SORB + NADH | BS095 | | |
| dehydrogenase | | | | | |
| iron-uptake system (binding protein) | feuA | IRONxt + ATP -> IRON + ADP + Pi | BS096 | | |
| (ABC Transport) | | | | | |
| 2-keto-3-deoxygluconate permease | kdgT | K3DGCxt + HEXT -> K3DGC | BS097 | | |
| lysine 2,3-aminomutase | kamA | LYS <-> DMHEX | BS098 | | |
| 6-phospho-alpha-glucosidase | malA | MAL6P -> GLC + G6P | BS099 | 3.2.1.122 | |
| malate-H+/Na+-lactate antiporter | mleN | MALxt + Hxt + NA + LAC <-> | BS100 | | |
| | | MAL + NAxt + LACxt | | | |
| Na+/malate symporter | maeN | MALxt + NAxt <-> MAL + NA | BS101 | | |
| unknown; similar to D-mannonate | yjmF | MANNT + NAD <-> FRCUR + NADH | BS102 | 1.1.1.57 | |
| oxidoreductase | | | | | |
| altronate hydrolase | uxaA | MANNT -> K3DGC + H2O | BS103 | 4.2.1.7 | |
| D-mannonate hydrolase | uxuA | MANNT -> K3DGC + H2O | BS104 | 4.2.1.7 | |
| manganese ABC transporter | mntA | MNxt + ATP -> MN + ADP + PI | BS105 | | |
| (membrane protein) | | | | | |
| Na+ ABC transporter (extrusion) (ATP- | natA | NA + ATP -> NAxt + ADP + Pi | BS106 | | |
| binding protein) | | | | | |
| ornithine acetyltransferase/amino-acid | argJ | NAARON + GLU -> ORN + NAGLU | BS107 | 2.3.1.35; | |
| acetyltransferase | | | | 2.3.1.1 | |
| unknown; similar to acetylornithine | ylmB2 | NAGMET -> AC + MET | BS108 | | |
| deacetylase | | | | | |
| unknown; similar to nitric-oxide | yojN | NADP + N2O + H2O -> 2 NO + NADPH | BS109 | | |
| reductase (assume acceptor = NADP) | | | | | |
| nitrate reductase (alpha subunit) | narG | NADPH + NO3 -> NADP + NO2 | BS110 | 1.7.99.4 | |
| assimilatory nitrate reductase (electron | nasB | NADPH + NO3 -> NADP + NO2 | BS111 | 1.6.6.4 | |
| transfer subunit) | | | | | |
| FMN-containing NADPH-linked | nfrA | NADPH + RIBFLV -> RIBFLVRD + | BS112 | 1.—.—.— | |
| nitro/flavin reductase | | NADP | | | |
| unknown; similar to NADPH-flavin | ycnD | NADPH + RIBFLV -> RIBFLVRD + | BS113 | | |
| oxidoreductase | | NADP | | | |
| oxalate decarboxylase | oxdC | OAA -> PYR + CO2 | BS114 | 4.1.1.3 | |
| unknown; similar to | yqiQ | PEP <-> 3PNPYR | BS115 | 5.4.2.9 | |
| phosphoenolpyruvate mutase | | | | | |
| unknown; similar to proline | yusM | PRO + FAD -> GLUGSAL + FADH | BS116 | | |
| dehydrogenase | | | | | |
| unknown; similar to pyruvate oxidase | ydaP | PYR + PI + O2 + H2O -> ACTP + | BS117 | 1.2.3.3 | |
| | | CO2 + H2O2 | | | |
| unknown; similar to ribulose- | ykrW | R15BP + CO2 -> 2 3PG | BS118 | 4.1.1.39 | |
| bisphosphate carboxylase | | | | | |
| unknown; similar to retinol | yusZ | retinol + NAD <-> retinal + NADH | BS119 | 1.1.1.105 | |
| dehydrogenase | | | | | |
| unknown; similar to methylglyoxalase | yurT | SLGT -> RGT + MTHGXL | BS120 | | |
| unknown; similar to mandelate | yitF | SMAND <-> RMAND | BS121 | | |
| racemase | | | | | |
| unknown; similar to sorbitol-6- | yuxG | SORB6P + NAD -> F6P + NADH | BS122 | 1.1.1.140 | |
| phosphate 2-dehydrogenase | | | | | |
| sorbitol dehydrogenase | gutB | SORBT + NAD -> SORB + NADH | BS123 | 1.1.1.14 | |
| squalene-hopene cyclase | sqhC | SQU -> HOP | BS124 | | |
| levansucrase | sacB | SUC + 26FRUCT -> GLC + 26FRUCT | BS125 | 2.4.1.10 | |
| sucrase-6-phosphate hydrolase | sacA | SUC6P -> SUC + PI | BS126 | 3.2.1.26 | |
| serine hydroxymethyltransferase | glyA | THF + SER <-> GLY + METTHF | BS127 | 2.1.2.1 | |
| pyrimidine-nucleoside transport protein | nupC5 | THYxt + HEXT -> THY | BS128 | | |
| UDP-glucose diacylglycerol | ugtP | UDPG + DGR -> UDP + GLCDG | BS129 | | |
| glucosyltransferase | | | | | |
| 1,4-alpha-glucan branching enzyme | glgB | UDPGLC -> UDP + GLYCOGEN | BS130 | 2.4.1.18 | |
| uricase | pucL | URATE + O2 -> ALLTN | BS131 | 1.7.3.3 | |
| uridine kinase | udk1 | URI + ATP -> ADP + UMP | BS132 | 2.7.1.48 | |
| uridine kinase | udk3 | URI + CTP -> CDP + UMP | BS133 | 2.7.1.48 | |
| pyrimidine-nucleoside phosphorylase | pdp2 | URI + R1P -> URA + PI | BS134 | 2.4.2.2 | |
| xanthine dehydrogenase | pucABCDE | XAN + NAD -> URATE + NADH | BS135 | | |
| xanthine phosphoribosyltransferase | xpt | XAN + PRPP -> RXAN5P + PPI | BS136 | 2.4.2.— | |

| Abbreviation | Metabolite |
|---|---|
| 13DPG | 1,3-bis-Phosphoglycerate |
| 23CAMP | nucleoside 2′,3′-cyclic phosphate |
| 23DACOA | 2,3-dehydroacyl-CoA |
| 23DHB | 2,3-Dihydroxybenzoate |
| 23DHBA | 2,3-Dihydroxybenzoyl-adenylate |
| 23DHDHB | 2,3-Dihydo-2,3-dihydroxybenzoate |
| 25DXP | 2,5-Dioxopentanoate |
| 26FRUCT | β-2,6-fructan |
| 2A3O | 2-Amino-3-oxobutanoate |
| 2D3D6PG | 2-Dehydro-3-deoxy-6-phospho-D-gluconate |
| 2DGLCN | 2-Deoxy-D-gluconate |
| 2KD6PG | 2-keto-3-deoxy-6-phospho-gluconate |
| 2M3OP | 2-methyl-3-oxopropanoate (Methylmalonate semialdehyde) |
| 2MAACOA | 2-Methyl-acetoacetyl-CoA |
| 2MBACP | 2-Methylbutanoyl-ACP |
| 2MBCOA | 2-Methylbutanoyl-CoA |
| 2MBECOA | trans-2-Methyl-but-2-enoyl-CoA |
| 2PG | 2-Phosphoglycerate |
| 2PGLYC | 2-phosphoglycolate |
| 34DHPHAC | 3,4-dihydroxyphenylacetate |
| 3AMP | nucleoside 3′-phosphate |
| 3D2DGLCN | 3-Dehydro-2-deoxy-D-gluconate |
| 3DDAH7P | 3-Deoxy-d-arabino heptulosonate-7-phosphate |
| 3DG25DS | 3-Deoxy-D-glycero-2,5-dexodiulosonate |
| 3H2MBCOA | (S)-3-Hydroxy-2-methyl-CoA |
| 3H2MP | 3-hydroxy-2-methylpropanoate |
| 3HBCOA | (S)-3-Hydroxy-isobutyryl-ACP |
| 3HIBCOA | (S)-3-Hydroxy-isobutyryl-CoA |
| 3HMGCOA | (S)-3-Hydroxy-3-methylglutaryl-CoA |
| 3M2ECOA | 3-Methylbut-2-enoyl-CoA |
| 3MBACP | 3-Methylbutanoyl-ACP |
| 3MBCOA | 3-Methylbutanoyl-CoA |
| 3MGCOA | 3-Methylglutaconyl-CoA |
| 3PG | 3-Phosphoglycerate |
| 3PNPYR | 3-phosphonopyruvate |
| 3PSER | 3-Phosphoserine |
| 3PSME | 3-Phosphate-shikimate |
| 3SALA | 3-sulfinoalanine |
| 4D5HSUR | 4-Deoxy-L-threo-5-hexosulose uronate |
| 4HPHAC | 4-hydroxyphenylacetate |
| 4IMZP | 4-imidazolone-5-propanoate |
| 4NPH | 4-nitrophenol |
| 4NPPI | 4-nitrophenyl phosphate |
| 4PPNCYS | 4′-Phosphopantothenoylcysteine |
| 4PPNTE | 4′-Phosphopantetheine |
| 4PPNTO | 4′-Phosphopantothenate |
| 5D4DGLCR | 5-Dehydro-4-deoxy-D-glucarate |
| 5DHGLCN | 5-dehydro-D-gluconate |
| 5MTA | 5-Methylthioadenosine |
| 5MTR | 5-Methylthio-D-ribose |
| 5MTR1P | 5-Methylthio-5-deoxy-D-ribulose 1-phosphate |
| 5MTRP | S5-Methyl-5-thio-D-ribose |
| 6PGG | 6-phospho-β-D-glucosyl-(1,4)-D-glucose |
| A6RP | 5-Amino-6-ribitylamino-2,4(1H,3H)-pyrimidinedione |
| A6RP5P | 5-Amino-6-(ribosylamino)-2,4-(1H,3H)-pyrimidinedione 5′-phosphate |
| A6RP5P2 | 5-Amino-2,6-dioxy-4-(5′-phosphoribitylamino)pyrimidine |
| AA | D-Alanyl-D-alanine |
| AAC | Acetoacetate |
| AACCOA | Acetoacetyl-CoA |
| ABUT | 2-Aceto-2-hydroxy butyrate |
| AC | Acetate |
| ACACP | Acetyl-ACP |
| ACAL | Acetaldehyde |
| ACARA | N-acetoxyarylamine |
| ACCOA | Acetyl-CoA |
| ACLAC | Acetolactate |
| ACMALT | acetyl-maltose |
| ACOA | Acyl-CoA |
| ACP | Acyl carrier protein |
| ACTN | Acetoin |
| ACTNxt | Acetoin external |
| ACTP | Acetyl-phosphate |
| AD | Adenine |
| ADCHOR | 4-Amino-4-deoxychorismate |
| ADN | Adenosine |
| ADNxt | Adenosine external |
| ADP | Adenosine diphosphate |
| ADPGLC | ADP-Glucose |
| ADPRIB | ADPRibose |
| AGM | Agmatine |
| AHHMD | 2-Amino-4-hydroxy-6-hydroxymethyl dihydropteridine-pp |
| AHHMP | 2-Amino-4-hydroxy-6-hydroxymethyl dihydropteridine |
| AHM | 4-Amino-5-hydroxymethyl-2-methylpyrimidine |
| AHMP | 4-Amino-5-hydroxymethyl-2-methylpyrimidine-phosphate |
| AHMPP | 4-Amino-5-hydroxymethyl-2-methylpyrimidine-pyrophosphate |
| AHTD | 2-Amino-4-hydroxy-6-(erythro-1-2-3-trihydroxypropyl) dihydropteridine-p |
| AICAR | 5-Phosphate-ribosyl-5-amino-4-imidazole carboxamide |
| AIR | 5-Phosphoribosyl-5-aminoimidazole |
| AKG | α-Ketoglutarate |
| AKP | α-Ketopantoate |
| ALA | Alanine |
| ALAV | D-Aminolevulinate |
| ALC | Alcohol |
| ALLTN | Allantoin |
| ALLTT | Allantoate |
| ALTRN | D-altronate |
| AMP | Adenosine monophosphate |
| AN | Antranilate |
| AONA | 8-Amino-7-oxononanoate |
| APPPPA | diadenosine tetraphospate |
| APS | Adenylyl sulfate |
| ARAB | Arabinose |
| ARG | Arginine |
| ARGSUCC | L-Arginio succinate |
| ASER | O-Acetylserine |
| ASN | Asparagine |
| ASP | Aspartate |
| ASPSA | Aspartic beta-semialdehyde |
| ASUC | Adenilsuccinate |
| ATP | Adenosine triphosphate |
| bALA | β-Alanine |
| BASP | β-Aspartyl phosphate |
| BCAA | Branched chain amino acid |
| bDG6P | β-D-Glucose 6-Phosphate |
| BENALD | Benzaldehyde |
| BENOH | Benzyl alcohol |
| BENZ | Benzoate |
| BT | Biotin |
| BUT | Butyrate |
| BUTCOA | Butanoyl-CoA |
| BUTN | Butanediol |
| BUTP | Butanoyl phosphate |
| C140IACP | Iso-C14:0-ACP |
| C140NACP | C14:0-ACP |
| C150AACP | Anteiso-C15:0-ACP |
| C150IACP | Iso-C15:0-ACP |
| C160IACP | Iso-C16:0-ACP |
| C160NACP | C16:0-ACP |
| C161IACP | Anteiso-C16:1-ACP |
| C161NACP | C16:1-ACP |
| C170AACP | Anteiso-C17:0-ACP |
| C170IACP | Iso-C17:0-ACP |
| C171AACP | Anteiso-C17:1-ACP |
| C171IACP | Iso-C17:1-ACP |
| C180NACP | C18:0-ACP |
| CAASP | Carbamoyl aspartate |
| CADV | Cadaverine |
| CAIR | 5-Phosphoribosyl-5-aminoimidazole-4-carboxylate |
| CAP | Carbamoyl phosphate |
| CBHCAP | 3-Carboxy-3-hydroxy-isocaproate |
| CDP | Cytidine diphosphate |
| CDP46GLC | CDP-4-dehydro-6-deoxy-D-glucose |
| CDPDG | CDP-1,2-Diacylglycerol |
| CDPGLC | CDP-Glucose |
| CDPGLYC | CDPglycerol |
| CH3SH | Methanethiol |
| CHCOA | 6-Carboxyhexanoyl-coa |
| CHOL | Choline |
| CHOR | Chorisimate |
| CHX | 6-Carboxyhexanoate |
| CIT | Citrate |
| CITR | L-Citrulline |
| CL | Cardiolypin |
| CMP | Cytidine monophosphate |
| CMRCOA | 4-coumaroyl-CoA |
| CO2 | Carbon dioxide |
| COA | Coenzyme A |
| CPAD5P | 1-O-Carboxyphenylamino 1-deoxyribulose-5-phosphate |
| CPP | Coproporphyrinogen III |
| CTP | Cytidine triphosphate |
| CYS | Cysteine |
| CYTD | Cytidine |
| CYTS | Cytosine |
| D23PIC | 2,3-Dihydro dipicolinate |
| D26PIM | L,I-2,6-Diamino pimelate |
| D6PGC | D-6-Phosphate-gluconate |
| D6PGL | D-6-Phosphate-glucono-delta-lactone |
| D6RP5P | 2,5-Diamino-6-(ribosylamino)-4-(3H)-pyrimidinone 5'-phosphate |
| D8RL | 6,7-Dimethyl-8-(1-D-ribityl)lumazine |
| DA | Deoxyadenosine |
| DADP | Deoxyadenosine diphosphate |
| DALA | D-Alanine |
| DAMP | Deoxyadenosine monophosphate |
| DANNA | 7,8-Diaminononanoate |
| DATP | Deoxyadenosine triphosphate |
| DB4P | 3,4-Dihydroxy-2-butanone-4-phosphate |
| DC | Deoxycytidine |
| DCDP | Deoxycytidine diphosphate |
| DCMP | Deoxycytidine monophosphate |
| DCTP | Deoxycytidine triphosphate |
| DG | Deoxyguanosine |
| DGDP | Deoxyguanosine diphosphate |
| DGLU | D-Glutamate |
| DGMP | 2-Deoxy-guanosine-5-phosphate |
| DGR | D-1,2-Diacylglycerol |
| DGTP | Deoxyguanosine triphosphate |
| DHF | Dihydrofolate |
| DHMVA | 2,3-Dihydroxy-3-methyl-valerate |
| DHNA | 1,4-Dihydroxy-2-naphthoic acid |
| DHP | Dihydroneopterin |
| DHPT | 7,8-Dihydropteroate |
| DHSK | Dehydroshikimate |
| DHVAL | Dihydroxy-isovalerate |
| DIACT | Diacetyl |
| DIMGP | D-Erythro imidazoleglycerol-phosphate |
| DIN | Deoxyinosine |
| DIPEP | Dipeptide |
| DKMPP | 2,3-Diketo-5-methylthio-1-phosphopentane |
| DMHEX | (3S)-3,6-diaminohexanoate |
| DMK | Demethylmenaquinone |
| DMPP | Dimethylallyl pyrophosphate |
| DOROA | Dihydroorotic acid |
| DPCOA | Dephosphocoenzyme A |
| DQT | 3-Dehydroquinate |
| DR1P | Deoxyribose 1-Phosphate |
| DR5P | Deoxyribose 5-Phosphate |
| DSAM | Decarboxylated adenosylmethionine |
| DSER | D-Serine |
| DT | Thymidine |
| DTB | Dethiobiotin |
| DTDP | Thymidine diphosphate |
| DTMP | Thymidine monophosphate |
| DTP | 1-Deoxy-d-threo-2-pentulose |
| DTTP | Thymidine triphosphate |
| DU | Deoxyuridine |
| DUDP | Deoxyuridine diphosphate |
| DUMP | Deoxyuridine monophosphate |
| DUTP | Deoxyuridine triphosphate |
| E4P | Erythrose 4-phosphate |
| ENTER | Enterochelin |
| EPOX | Epoxide |
| ETH | Ethanol |
| F1P | Fructose 1-Phosphate |
| F6P | Fructose 6-phosphate |
| FAD | Flavin adenine dinucleotide |
| FADH | Flavin adenine dinucleotide reduced |
| FAM | formamide |
| FDP | Fructose 1,6-diphosphate |
| FGAM | 5-Phosphoribosyl-n-formylgycineamidine |
| FGAR | 5-Phosphoribosyl-n-formylglycineamide |
| FMN | flavin mononucleotide |
| FOR | Formate |
| FORMALD | Formaldehyde |
| FPP | trans, trans Farnesyl pyrophosphate |
| FRCUR | D-fructuronate |
| FRU | Fructose |
| FTHF | 10-formyl-tetrahydrofolate |
| FUM | Fumarate |
| G15LAC | D-glucono-1,5-lactone |
| G16DP | Glucose 1,6-diphosphate |
| G1P | Glucose 1-phosphate |
| G6P | Glucose 6-phosphate |
| GA1P | Glucosamine 1-phosphate |
| GA6P | D-Glucosamine |
| GABA | 4-Aminobutanoate |
| GAL1P | Galactose 1-Phosphate |
| GALUR | D-galacturonate |
| GAR | 5-Phosphate-ribosyl glycineamide |
| GDP | Guanosine diphosphate |
| GDPTP | guanosine 3'-diphosphate 5'-triphosphate |
| GL | Glycerol |
| GL3P | Glycerol 3-phosphate |
| GLAC | Galactose |
| GLAL | D-Glyceraldehyde |
| GLC | α-D-Glucose |
| GLCAM6P | glucosamine 6-phosphate |
| GLCDG | 3-D-glucosyl-1,2-diacylglycerol |
| GLCN | Gluconate |
| GLCR | (R)-glycerate |
| GLCUR | D-glucuronate |
| GLN | Glutamine |
| GLT6P | Glucitol 6-Phosphate |
| GLU | Glutamate |
| GLUGSAL | 1-pyrroline-5-carboxylate |
| GLUP | Glutamyl phosphate |
| GLX | Glyoxylate |
| GLY | Glycine |
| GLYBET | Glycine Betaine |
| GLYC | glycolate |
| GLYCOGEN | Glycogen |
| GLYCOL | Glycol |
| GLYPD | glycerophosphodiester |
| GLYTC1 | D-alanyl glycerol teichoic acid |
| GLYTC2 | glucosyl glycerol teichoic acid |
| GMP | Guanosine monophosphate |
| GN | Guanine |
| GPP | trans Geranyl pyrophosphate |
| GSA | Glutamate-1-semialdehyde |
| GSN | Guanosine |
| GTP | Guanosine triphosphate |
| GTRNA | L-Glutamyl-tRNA(glu) |
| H2CO3 | Carbonate |
| H2O | Water |
| H2O2 | Hydrogen Peroxide |
| H2S | Hydrogen sulfide |
| H2SO3 | Sulfite |
| H2SO4 | Sulfate |
| HACOA | Hydroxyacyl-CoA |
| HCYS | Homocysteine |
| HEMEA | Heme A |
| HEXT | External H+ |
| HIPP | hippurate |
| HIS | Histidine |
| HISOL | Histidinol |
| HISOLP | L-Histidinol-phosphate |
| HMB | Hydroxymethylbilane |
| HO | Heme O |
| HOP | Hopene |
| HPHPYR | para-Hydroxy phenyl pyruvate |
| HPYR | hydroxypyruvate |
| HSER | Homoserine |
| HTPP | heptaprenyl diphosphate |
| HXARA | N-Hydroxyarylamine |
| HXPP | hexaprenyl diphosphate |
| HYXN | Hypoxanthine |
| ICHOR | Isochorismate |
| ICIT | Isocitrate |
| IDITOL | L-Iditol |
| IDP | Inosine diphosphate |
| IGP | Indole glycerol phosphate |
| ILE | Isoleucine |
| IMACP | Imidazole acetyl-phosphate |
| IMP | Inosine monophosphate |
| INOSIT | Inositol |
| INS | Inosine |
| IPPMAL | 3-Isopropylmalate |
| IPPP | Isopentyl pyrophosphate |
| IRON | IRON |
| ISBACP | Isobutyryl-ACP |
| ISBCOA | Isobutyryl-CoA |
| ISUCC | α-iminosuccinate |
| ITP | Inosine triphosphate |
| K | Potassium |
| K3DGC | 2-keto-3-deoxygluconate |
| KMB | α-keto-g-methiobutyrate |
| LAC | D-Lactate |
| LACAL | Lactaldehyde |
| LCCA | Long-chain carboxylic acid |
| LCTS | Lactose |
| LEU | Leucine |
| LLAC | L-Lactate |
| LLCT | L-Cystathionine |
| LRL5P | L-Ribulose 5-phosphate |
| LYS | L-Lysine |
| MAL | Malate |
| MAL6P | Maltose 6-phosphate |
| MALACP | Malonyl-ACP |
| MALCOA | Malonyl-CoA |
| MALT | Maltose |
| MAN1P | Mannose 1-Phosphate |
| MAN6P | Mannose 6-Phosphate |
| MANNT | Mannonate |
| MCCOA | Methacrylyl-CoA |
| MDAP | Meso-diaminopimelate |
| MELI | Melibiose |
| MET | Methionine |
| METHF | 5,10-Methenyl tetrahydrofolate |
| METTHF | 5,10-Methylene tetrahydrofolate |
| MLT6P | Maltose 6-phosphate |
| MN | Manganese |
| MNT6P | Mannitol 6-Phosphate |
| MTHF | 5-Methyl tetrahydrofolate |
| MTHGXL | Methylglyoxal |
| N2O | Nitrous Oxide |
| NA | Sodium |
| NAAD | Nicotinic acid adenine dinucleotide |
| NAARON | N-α-Acetyl omithine |
| NACMET | N-acetylmethionine |
| NAD | Nicotinamide adenine dinucleotide |
| NADH | Nicotinamide adenine dinucleotide reduced |
| NADP | Nicotinamide adenine dinucleotide phosphate |
| NADPH | Dihydronicotinamide adenine dinucleotide phosphate reduced |
| NAG | N-Acetylglucosamine |
| NAGLU | N-Acetyl glutamate |
| NAGLUSAL | N-Acetyl glutamate semialdehyde |
| NAGLUYP | N-Acetyl glutamyl-phosphate |
| NAGP | N-Acetylglucosamine (6-phosphate) |
| NAMN | Nicotinic acid mononucleotide |
| NARGC | naringenin-chalcone |
| NCAIR | 5'-Phosphoribosyl-5-carboxyaminoimidazole |
| NFGLU | N-formimidoyl-L-glutamate |
| NH3 | Ammonia |
| NMN | Nicotinamide mononucleotide |
| NO | Nitric Oxide |
| NO2 | Nitrite |
| NO3 | Nitrate |
| NPRAN | N-5-phosphoribosyl-antranilate |
| NS26DP | N-Succinyl-I,I-2,6-diaminopimelate |
| NS2A6O | N-Succinyl-2-amino-6-ketopimelate |
| O2 | Oxygen |
| OA | Oxaloacetate |
| OACOA | 3-Oxoacyl-CoA |
| OASER | O-acetyl-L-serine |
| OBUT | Oxobutyrate |
| OICAP | 2-Oxoisocaproate |
| OIVAL | 3-Methyl-2-oxobutanoate (2-Oxoisovalerate) |
| OMP | Orotidylate |
| OMVAL | 3-Methyl-2-oxopentanoate (OMVAL) |
| OPEP | Oligopeptide |
| OPP | trans Octaprenyl pyrophosphate |
| ORN | Ornithine |
| OROA | Orotic acid |
| OSB | O-Succinylbenzoic acid |
| OSBCOA | O-Succinylbenzoyl-CoA |
| OSLHSER | O-Succinyl-I-homoserine |
| OTHIO | Thioredoxin (oxidized form) |
| PA | Phosphatidyl acid |
| PABA | para-Aminobenzoic acid |
| PANT | Pantoate |
| PAP | Adenosine-3',5'-diphosphate |
| PAPS | 3-Phosphoadenylyl sulfate |
| PBG | Probilinogen III |
| PC2 | Percorrin 2 |
| PE | Phosphatidyl ethanolamine |
| PEP | Phosphoenolpyruvate |
| PEPT | Peptide |
| PEPTIDO | Peptidoglycan |
| PG | Phosphatidyl glycerol |
| PGP | L-1-Phoshatidyl-glycerol-phosphate |
| PHE | Phenylalanine |
| PHEN | Prephenate |
| PHP | 3-Phosphohydroxypyruvate |
| PHPYR | Phenyl pyruvate |
| PHSER | O-Phospho-I-homoserine |
| PI | Phosphate (inorganic) |
| PIP26DX | Delta-piperidine-2,6-dicarboxylate |
| PNTO | Pantothenate |
| PPCOA | propanoyl-CoA |
| PPHG | Protoporphyrinogen |
| PPI | Pyrophosphate |
| PPIX | Protoporphyrin IX |
| PRAM | 5-Phosphate-β-D-ribosyl amine |
| PRBAMP | Phosphoribosyl-AMP |
| PRBATP | Phosphoribosyl-ATP |
| PRFICA | 5-Phosphate-ribosyl-formamido-4-imidazole carboxamide |
| PRFP | Phosphoribosyl-formimino-AICAR-phosphate |
| PRLP | Phosphoribulosyl-formimino-AICAR-phosphate |
| PRO | Proline |
| PRPP | Phosphoribosyl pyrophosphate |
| PS | Phosphatidyl serine |
| PTH | Protoheme |
| PTRC | Putrescine |
| PYR | Pyruvate |
| Q | Menaquinone |
| QA | Quinolinate |
| QH2 | Ubiquinol |
| R15BP | D-ribulose 1,5-bisphosphate |
| R1P | Ribose 1-phosphate |
| R5P | Ribose 5-phosphate |
| RBL | Ribulose |
| retinal | Retinal |
| retinol | Retinol |
| RGT | Reduced glutathione |
| RIB | Ribose |
| RIBFLV | Riboflavin |
| RIBFLVRD | Riboflavin reduced |
| RL5P | Ribulose 5-phosphate |
| RMAND | (R)-mandelate |
| RML | Rhamnulose |
| RML1P | Rhamnulose 1-phosphate |
| RMMCOA | (R)-methylmalonyl-CoA |
| RMN | Rhamnose |
| RTHIO | Thioredoxin (reduced form) |
| RXAN5P | (9-D-ribosylxanthine)-5'-phosphate |
| S7P | sedo-Heptulose |
| SAH | S-Adenosyl homocystine |
| SAICAR | 5-Phosphoribosyl-4-(N-succinocarboxyamide)-5-amino-imidazole |
| SAM | S-Adenosyl methionine |
| SAMOB | S-Adenosyl-4-methylthio-2-oxobutanoate |
| SER | Serine |
| SERA | L-Seryl-adenylate |
| SHCHC | 2-Succinyl-6-hydroxy-2,4-cyclohexadiene-1-carboxylate |
| SHCL | Sirohydrochlorin |
| SHEME | Siroheme |
| SLGT | (R)-S-lactoylglutathione |
| SMAND | (S)-mandelate |
| SME | Shikimate |
| SME5P | Shikimate-5-phosphate |
| SMMCOA | (S)-methylmalonyl-CoA |
| SORB | Sorbose |
| SORB6P | D-sorbitol 6-phosphate |
| SORBT | Sorbitol |
| SPMD | Spermidine |
| SQU | Squalene |
| SSALTPP | Succinate semialdehyde —thiamine pyrophosphate |
| SUC | Sucrose |
| SUC6P | Surose 6-phosphate |
| SUCC | Succinate |
| SUCCOA | Succinate CoA |
| SUCCSAL | Succinate semialdehyde |
| T3P1 | Glyceraldehyde 3-phosphate |
| T3P2 | Dihydroxyacetone-phosphate |
| TAGATU | D-tagaturonate |
| TEICHU | Teichuronic Acid |
| THF | Tetrahydrofolate |
| THIAMIN | Thiamin |
| THMP | Thiamine-phosphate |
| THR | Threonine |
| THY | Thymine |
| THZ | 4-Methyl-5-(beta-hydroxyethyl)thiazole |
| THZP | 4-Methyl-5-(beta-hydroxyethyl)thiazole phosphate |
| TPP | Thiamine-pyrophosphate |
| TRE6P | Trehalose 6-phosphate |
| TRP | Tryptophan |
| TYR | Tyrosine |
| UDP | Uridine diphosphate |
| UDPG | UDP Glucose |
| UDPGAL | UDP Galactose |
| UDPGCU | UDP-Glucouronate |
| UDPNAG | UDP N-acetyl glucosamine |
| UDPNAGAL | UDP-N-acetyl-Galactosamine |
| UDPNAGEP | UDP-N-acetyl-3-O-(1-carboxyvinyl)-D-glucosamine |
| UDPNAM | UDP-N-acetyl-D-muramate |
| UDPNAMA | UDP-N-acetylmuramoyl-L-alanine |
| UDPNAMAG | UDP-N-acetylmuramoyl-L-alanyl-D-glutamate |
| UDPNAMS | UDP-N-acetyl-Mannosamine |
| UDPP | Undecaprenyl pyrophosphate |
| UMP | Uridine monophosphate |
| UNAGD | UDP-N-acetylmuramoyl-L-alanyl-D-glutamyl-meso-2,6-diaminoheptanedioate |
| UNAGDA | UDP-N-acetylmuramoyl-L-alanyl-D-glutamyl-meso-2,6-diaminoheptanedioate-D-alanyl-D-alanine |
| UNPTDO | UDP-N-acetylmuramoyl-L-alanyl-D-glutamyl-meso-2,6-diaminoheptanedioate-D-alanyl-D-alanine- |
| | diphosphoundecaprenol |
| UPRG | Uroporphyrinogen III |
| URA | Uracil |
| URATE | Urate |
| URCAN | urocanate |
| UREA | Urea |
| URI | Uridine |
| UTP | Uridine triphosphate |
| VAL | Valine |
| X5P | Xylulose-5-phosphate |
| XAN | Xanthine |
| XMP | Xantosine monophosphate |
| XTSN | Xanthosine |
| XUL | Xylulose |
| XYL | Xylose |

What is claimed is:

1. A computer readable medium or media, comprising:
(a) a data structure relating a plurality of Bacillus subtilis reactants to a plurality of Bacillus subtilis reactions,
wherein each of said Bacillus subtilis reactions comprises a reactant identified as a substrate of the reaction, a reactant identified as a product of the reaction and a stoichiometric coefficient relating said substrate and said product,
wherein at least one of said Bacillus subtilis reactions is annotated to indicate an associated gene;
(b) a gene database comprising information characterizing said associated gene;
(c) a constraint set for said plurality of Bacillus subtilis reactions, and
(d) commands for determining at least one flux distribution that minimizes or maximizes an objective function when said constraint set is applied to said data representation, wherein said at least one flux distribution is predictive of a Bacillus subtilis physiological function.

2. The computer readable medium or media of claim 1, wherein said plurality of Bacillus subtilis reactions comprises at least one reaction from a peripheral metabolic pathway.

3. The computer readable medium or media of claim 2, wherein said peripheral metabolic pathway is selected from the group consisting of amino acid biosynthesis, amino acid degradation, purine biosynthesis, pyrimidine biosynthesis, lipid biosynthesis, fatty acid metabolism, cofactor biosynthesis, cell wall metabolism and transport processes.

4. The computer readable medium or media of claim 1, wherein said Bacillus subtilis physiological function is selected from the group consisting of growth, energy production, redox equivalent production, biomass production, production of biomass precursors, production of a protein, production of an amino acid, production of a purine, production of a pyrimidine, production of a lipid, production of a fatty acid, production of a cofactor, production of a cell wall component, transport of a metabolite, and consumption of carbon nitrogen, sulfur, phosphate, hydrogen or oxygen.

5. The computer readable medium or media of claim 1, wherein said Bacillus subtilis physiological function is selected from the group consisting of degradation of a protein, degradation of an amino acid, degradation of a purine, degradation of a pyrimidine, degradation of a lipid, degradation of a fatty acid, degradation of a cofactor and degradation of a cell wall component.

6. The computer readable medium or media of claim 1, wherein said data structure comprises a set of linear algebraic equations.

7. The computer readable medium or media of claim 1, wherein said data structure comprises a matrix.

8. The computer readable medium or media of claim 1, wherein said commands comprise an optimization problem.

9. The computer readable medium or media of claim 1, wherein said commands comprise a linear program.

10. The computer readable medium or media of claim 1, wherein at least one reactant in said plurality of Bacillus subtilis reactants or at least one reaction in said plurality of Bacillus subtilis reactions is annotated with an assignment to a subsystem or compartment.

11. The computer readable medium or media of claim 10, wherein a first substrate or product in said plurality of Bacillus subtilis reactions is assigned to a first compartment and a second substrate or product in said plurality of Bacillus subtilis reactions is assigned to a second compartment.

12. The computer readable medium or media of claim 1, wherein a plurality of said Bacillus subtilis reactions is annotated to indicate a plurality of associated genes and wherein said gene database comprises information characterizing said plurality of associated genes.

13. A computer readable medium or media, comprising:
(a) a data structure relating a plurality of Bacillus subtilis reactants to a plurality of Bacillus subtilis reactions,
wherein each of said Bacillus subtilis reactions comprises a reactant identified as a substrate of the reaction, a reactant identified as a product of the reaction and a stoichiometric coefficient relating said substrate-and said product,
wherein at least one of said Bacillus subtilis reactions is a regulated reaction;
(b) a constraint set for said plurality of Bacillus subtilis reactions, wherein said constraint set includes a variable constraint for said regulated reaction, and
(c) commands for determining at least one flux distribution that minimizes or maximizes an objective function when said constraint set is applied to said data representation, wherein said at least one flux distribution is predictive of a Bacillus subtilis physiological function.

14. The computer readable medium or media of claim 13, wherein said variable constraint is dependent upon the outcome of at least one reaction in said data structure.

15. The computer readable medium or media of claim 13, wherein said variable constraint is dependent upon the outcome of a regulatory event.

16. The computer readable medium or media of claim 13, wherein said variable constraint is dependent upon time.

17. The computer readable medium or media of claim 13, wherein said variable constraint is dependent upon the presence of a biochemical reaction network participant.

18. The computer readable medium or media of claim 17, wherein said participant is selected from the group consisting of a substrate, product, reaction, protein, macromolecule, enzyme and gene.

19. The computer readable medium or media of claim 13, wherein a plurality of said reactions are regulated reactions and said constraints for said regulated reactions comprise variable constraints.

20. A computer readable medium or media, comprising:
(a) a data structure relating a plurality of Bacillus subtilis reactants to a plurality of Bacillus subtilis reactions,
wherein each of said Bacillus subtilis reactions comprises a reactant identified as a substrate of the reaction, a reactant identified as a product of the reaction and a stoichiometric coefficient relating said substrate and said product;
(b) a constraint set for said plurality of Bacillus subtilis reactions, and
(c) commands for determining at least one flux distribution that minimizes or maximizes an objective function when said constraint set is applied to said data representation, wherein said at least one flux distribution is predictive of Bacillus subtilis growth.

21. A method for predicting a Bacillus subtilis physiological function, comprising:
(a) providing a data structure relating a plurality of Bacillus subtilis reactants to a plurality of Bacillus subtilis reactions,
wherein each of said Bacillus subtilis reactions comprises a reactant identified as a substrate of the reaction, a reactant identified as a product of the reaction and a stoichiometric coefficient relating said substrate and said product,
wherein at least one of said Bacillus subtilis reactions is annotated to indicate an associated gene;
(b) providing a constraint set for said plurality of Bacillus subtilis reactions;
(c) providing an objective function, and
(d) determining at least one flux distribution that minimizes or maximizes said objective function when said constraint set is applied to said data structure, thereby predicting a Bacillus subtilis physiological function related to said gene.

22. The method of claim 21, wherein said plurality of Bacillus subtilis reactions comprises at least one reaction from a peripheral metabolic pathway.

23. The method of claim 22, wherein said peripheral metabolic pathway is selected from the group consisting of amino acid biosynthesis, amino acid degradation, purine biosynthesis, pyrimidine biosynthesis, lipid biosynthesis, fatty acid metabolism, cofactor biosynthesis, cell wall metabolism and transport processes.

24. The method of claim 21, wherein said Bacillus subtilis physiological function is selected from the group consisting of growth, energy production, redox equivalent production, biomass production, production of biomass precursors, production of a protein, production of an amino acid, production of a purine, production of a pyrimidine, production of a lipid, production of a fatty acid, production of a cofactor, production of a cell wall component, transport of a metabolite, and consumption of carbon nitrogen, sulfur, phosphate, hydrogen or oxygen.

25. The method of claim 21, wherein said Bacillus subtilis physiological function is selected from the group consisting of glycolysis, the TCA cycle, pentose phosphate pathway, respiration, biosynthesis of an amino acid, degradation of an amino acid, biosynthesis of a purine, biosynthesis of a pyrimidine, biosynthesis of a lipid, metabolism of a fatty acid, biosynthesis of a cofactor, metabolism of a cell wall component, transport of a metabolite and metabolism of a carbon source, nitrogen source, oxygen source, phosphate source, hydrogen source or sulfur source.

26. The method of claim 21, wherein said data structure comprises a set of linear algebraic equations.

27. The method of claim 21, wherein said data structure comprises a matrix.

28. The method of claim 21, wherein said flux distribution is determined by linear programming.

29. The method of claim 21, further comprising:
(e) providing a modified data structure, wherein said modified data structure comprises at least one added reaction, compared to the data structure of part (a), and
(f) determining at least one flux distribution that minimizes or maximizes said objective function when said constraint set is applied to said modified data structure, thereby predicting a Bacillus subtilis physiological function.

30. The method of claim 29, further comprising identifying at least one participant in said at least one added reaction.

31. The method of claim 30, wherein said identifying at least one participant comprises associating a Bacillus subtilis protein with said at least one reaction.

32. The method of claim 31, further comprising identifying at least one gene that encodes said protein.

33. The method of claim 30, further comprising identifying at least one compound that alters the activity or amount of said at least one participant, thereby identifying a candidate drug or agent that alters a Bacillus subtilis physiological function.

34. The method of claim 21, further comprising:
(e) providing a modified data structure, wherein said modified data structure lacks at least one reaction compared to the data structure of part (a), and
(f) determining at least one flux distribution that minimizes or maximizes said objective function when said constraint set is applied to said modified data structure, thereby predicting a Bacillus subtilis physiological function.

35. The method of claim 34, further comprising identifying at least one participant in said at least one reaction.

36. The method of claim 35, wherein said identifying at least one participant comprises associating a Bacillus subtilis protein with said at least one reaction.

37. The method of claim 36, further comprising identifying at least one gene that encodes said protein that performs said at least one reaction.

38. The method of claim 35, further comprising identifying at least one compound that alters the activity or amount of said at least one participant, thereby identifying a candidate drug or agent that alters a Bacillus subtilis physiological function.

39. The method of claim 21, further comprising:
(e) providing a modified constraint set, wherein said modified constraint set comprises a changed constraint for at least one reaction compared to the constraint for said at least one reaction in the data structure of part (a), and
(f) determining at least one flux distribution that minimizes or maximizes said objective function when said modified constraint set is applied to said data structure, thereby predicting a Bacillus subtilis physiological function.

40. The method of claim 39, further comprising identifying at least one participant in said at least one reaction.

41. The method of claim 40, wherein said identifying at least one participant comprises associating a Bacillus subtilis protein with said at least one reaction.

42. The method of claim 41, further comprising identifying at least one gene that encodes said protein.

43. The method of claim 40, further comprising identifying at least one compound that alters the activity or amount of said at least one participant, thereby identifying a candidate drug or agent that alters a Bacillus subtilis physiological function.

44. The method of claim 21, further comprising providing a gene database relating one or more reactions in said data structure with one or more genes or proteins in Bacillus subtilis.

45. A method for predicting a Bacillus subtilis physiological function, comprising:
(a) providing a data structure relating a plurality of Bacillus subtilis reactants to a plurality of Bacillus subtilis reactions,
wherein each of said Bacillus subtilis reactions comprises a reactant identified as a substrate of the reaction, a reactant identified as a product of the reaction and a stoichiometric coefficient relating said substrate and said product,
wherein at least one of said Bacillus subtilis reactions is a regulated reaction;
(b) providing a constraint set for said plurality of Bacillus subtilis reactions, wherein said constraint set includes a variable constraint for said regulated reaction;
(c) providing a condition-dependent value to said variable constraint;
(d) providing an objective function, and
(e) determining at least one flux distribution that minimizes or maximizes said objective function when said constraint set is applied to said data structure, thereby predicting a Bacillus subtilis physiological function.

46. The method of claim 45, wherein said value provided to said variable constraint changes in response to the outcome of at least one reaction in said data structure.

47. The method of claim 45, wherein said value provided to said variable constraint changes in response to the outcome of a regulatory event.

48. The method of claim 45, wherein said value provided to said variable constraint changes in response to time.

49. The method of claim 45, wherein said value provided to said variable constraint changes in response to the presence of a biochemical reaction network participant.

50. The method of claim 49, wherein said participant is selected from the group consisting of a substrate, product, reaction, enzyme, protein, macromolecule and gene.

51. The method of claim 45, wherein a plurality of said reactions are regulated reactions and said constraints for said regulated reactions comprise variable constraints.

52. A method for predicting Bacillus subtilis growth, comprising:
(a) providing a data structure relating a plurality of Bacillus subtilis reactants to a plurality of Bacillus subtilis reactions,
wherein each of said Bacillus subtilis reactions comprises a reactant identified as a substrate of the reaction, a reactant identified as a product of the reaction and a stoichiometric coefficient relating said substrate and said product;
(b) providing a constraint set for said plurality of Bacillus subtilis reactions;
(c) providing an objective function, and
(d) determining at least one flux distribution that minimizes or maximizes said objective function when said constraint set is applied to said data structure, thereby predicting Bacillus subtilis growth.

53. A method for making a data structure relating a plurality of Bacillus subtilis reactants to a plurality of Bacillus subtilis reactions in a computer readable medium or media, comprising:
(a) identifying a plurality of Bacillus subtilis reactions and a plurality of Bacillus subtilis reactants that are substrates and products of said Bacillus subtilis reactions;
(b) relating said plurality of Bacillus subtilis reactants to said plurality of Bacillus subtilis reactions in a data structure,
wherein each of said Bacillus subtilis reactions comprises a reactant identified as a substrate of the reaction, a reactant identified as a product of the reaction and a stoichiometric coefficient relating said substrate and said product;
(c) determining a constraint set for said plurality of Bacillus subtilis reactions;
(d) providing an objective function;
(e) determining at least one flux distribution that minimizes or maximizes said objective function when said constraint set is applied to said data structure, and
(f) if said at least one flux distribution is not predictive of a Bacillus subtilis physiological function, then adding a reaction to or deleting a reaction from said data structure and repeating step (e),
if said at least one flux distribution is predictive of a Bacillus subtilis physiological function, then storing said data structure in a computer readable medium or media.

54. The method of claim 53, wherein a reaction in said data structure is identified from an annotated genome.

55. The method of claim 54, further comprising storing said reaction that is identified from an annotated genome in a gene database.

56. The method of claim 53, further comprising annotating a reaction in said data structure.

57. The method of claim 56, wherein said annotation is selected from the group consisting of assignment of a gene, assignment of a protein, assignment of a subsystem, assignment of a confidence rating, reference to genome annotation information and reference to a publication.

58. The method of claim 53, wherein step (b) further comprises identifying an unbalanced reaction in said data structure and adding a reaction to said data structure, thereby changing said unbalanced reaction to a balanced reaction.

59. The method of claim 53, wherein said adding a reaction comprises adding a reaction selected from the group consisting of an intra-system reaction, an exchange reaction, a reaction from a peripheral metabolic pathway, reaction from a central metabolic pathway, a gene associated reaction and a non-gene associated reaction.

60. The method of claim 59, wherein said peripheral metabolic pathway is selected from the group consisting of amino acid biosynthesis, amino acid degradation, purine biosynthesis, pyrimidine biosynthesis, lipid biosynthesis, fatty acid metabolism, cofactor biosynthesis, cell wall metabolism and transport processes.

61. The method of claim 53, wherein said Bacillus subtilis physiological function is selected from the group consisting of growth, energy production, redox equivalent production, biomass production, production of biomass precursors, production of a protein, production of an amino acid, production of a purine, production of a pyrimidine, production of a lipid, production of a fatty acid, production of a cofactor, production of a cell wall component, transport of a metabolite, development, intercellular signaling, and consumption of carbon nitrogen, sulfur, phosphate, hydrogen or oxygen.

62. The method of claim 53, wherein said Bacillus subtilis physiological function is selected from the group consisting of degradation of a protein, degradation of an amino acid, degradation of a purine, degradation of a pyrimidine, degradation of a lipid, degradation of a fatty acid, degradation of a cofactor and degradation of a cell wall component.

63. The method of claim 53, wherein said data structure comprises a set of linear algebraic equations.

64. The method of claim 53, wherein said data structure comprises a matrix.

65. The method of claim 53, wherein said flux distribution is determined by linear programming.

66. A data structure relating a plurality of Bacillus subtilis reactants to a plurality of Bacillus subtilis reactions, wherein said data structure is produced by a process comprising:
(a) identifying a plurality of Bacillus subtilis reactions and a plurality of Bacillus subtilis reactants that are substrates and products of said Bacillus subtilis reactions;
(b) relating said plurality of Bacillus subtilis reactants to said plurality of Bacillus subtilis reactions in a data structure,
wherein each of said Bacillus subtilis reactions comprises a reactant identified as a substrate of the reaction, a reactant identified as a product of the reaction and a stoichiometric coefficient relating said substrate and said product;
(c) determining a constraint set for said plurality of Bacillus subtilis reactions;
(d) providing an objective function;
(e) determining at least one flux distribution that minimizes or maximizes said objective function when said constraint set is applied to said data structure, and
(f) if said at least one flux distribution is not predictive of Bacillus subtilis physiology, then adding a reaction to or deleting a reaction from said data structure and repeating step (e),
if said at least one flux distribution is predictive of Bacillus subtilis physiology, then storing said data structure in a computer readable medium or media.
